(19) United States
(12) Patent Application Publication
(10) Pub. No.: US 2025/0177514 A1
(43) Pub. Date: Jun. 5, 2025

(54) CORONAVIRUS ANTIGEN VARIANTS

(71) Applicant: PFIZER INC., NEW YORK, NY (US)

(72) Inventors: Ye CHE, Niantic, CT (US); Kena Anne SWANSON, Pearl River, NY (US)

(73) Assignee: PFIZER INC., NEW YORK, NY (US)

(21) Appl. No.: 18/833,823

(22) PCT Filed: Jan. 27, 2023
(86) PCT No.: PCT/IB2023/050744

§ 371 Date: Jul. 26, 2024

Related U.S. Application Data
(60) Provisional application No. 63/304,560, filed on Jan. 28, 2022.
(60) Provisional application No. 63/480,375, filed on Jan. 18, 2023.

(51) Int. Cl. A61K 39/215; A61K 9/127; A61K 9/51; A61K 39/00; C07K 14/005; C12N 7/00

(52) U.S. Cl. CPC A61K 39/215; A61K 9/127; A61K 9/5123; C07K 14/005; C12N 7/00; A61K 2039/55555; C12N 2770/20022; C12N 2770/20034

(57) ABSTRACT

The present disclosure provides isolated immunogenic polypeptides comprising variant coronavirus spike proteins that are variants of a native coronavirus spike protein or fragment thereof, the variant coronavirus spike proteins having one or more modifications compared to the native coronavirus spike protein that: increase adoption by a receptor binding domain (RBD) of the variant coronavirus spike protein of an up conformation; and/or decrease adoption by a RBD of the variant coronavirus spike protein of a down conformation; and/or increase expression of the variant coronavirus spike protein compared to the native coronavirus spike protein; and/or increase adoption of a prefusion conformation; and/or decrease shedding of a S1 subunit of the variant coronavirus spike protein; and/or improve localization of the variant coronavirus spike protein to a host cell membrane. The present disclosure also provides compositions comprising the isolated immunogenic polypeptides and methods of making and using such compositions.

### CROSS-REFERENCE TO RELATED APPLICATIONS

This application claims priority to U.S. provisional application No. 63/304,560, filed Jan. 28, 2022, and U.S. provisional application No. 63/480,375, filed Jan. 18, 2023, each of which is incorporated by reference herein in its entirety.

### BACKGROUND

### I. Technical Field

This disclosure relates to the field of immunology, specifically to immunogenic compositions. More particularly, it concerns methods and compositions involving coronavirus spike protein variants, which can be used to invoke an immune response against coronaviruses.

### II. Background

Coronaviruses are a large family of viruses that usually cause mild to moderate upper-respiratory tract illnesses, like the common cold. However, three new coronaviruses, MERS-CoV, SARS-CoV, and SARS-CoV-2, have emerged from animal reservoirs over the past two decades to cause serious and widespread illness and death. SARS coronavirus (SARS-CoV) emerged in November 2002 and caused severe acute respiratory syndrome (SARS). Middle East respiratory syndrome (MERS) is caused by the MERS coronavirus (MERS-CoV). Transmitted from an animal reservoir in camels, MERS was identified in September 2012 and continues to cause sporadic and localized outbreaks. The third novel coronavirus to emerge in this century is called SARS-CoV-2. It causes coronavirus disease 2019 (COVID-19), which emerged from China in December 2019 and was declared a global pandemic by the World Health Organization on Mar. 11, 2020.

Coronavirus infection is mediated by the receptor binding domain (RBD) of the coronavirus spike glycoprotein binding to the ACE2 receptor on the surface of a host cell membrane. Although the coronavirus spike sequence and structure are known and several immunogenic compositions exist to elicit an immune response to the spike, there remains a need for spike protein antigens with increased protein expression, stability, and immunogenic conformations as compared to presently-used immunogenic compositions in order to more effectively stimulate a protective immune response against coronaviruses.

### SUMMARY

The present disclosure provides methods and compositions involving coronavirus spike protein variants. The variants may invoke an immune response against coronaviruses more effectively than naturally occurring coronavirus spike proteins. Increasing the immunogenicity of viral antigens by, e.g., improving expression of the viral antigens, improving the stability of the viral antigens, and/or increasing the number of neutralization-sensitive epitopes on the viral antigens, is a desirable outcome in the safety and efficacy of vaccines. The present disclosure is based, at least in part, on the discovery that one or more specific amino acid modifications can be made to native coronavirus spike protein sequences to produce variant coronavirus spike proteins having improved in vivo expression, improved stability of the prefusion conformation, and/or increased exposure of neutralization-sensitive epitopes that may result in a more immunogenic antigen.

It is contemplated that any aspect discussed in this specification can be implemented with respect to any method or composition of the disclosure, and vice versa. Furthermore, compositions of the disclosure can be used to achieve methods of the disclosure.

Other objects, features and advantages of the present disclosure will become apparent from the following detailed description. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific aspects presented herein.

FIG. 1 is a schematic of the various domains of a coronavirus spike protein according to some aspects disclosed herein.

FIG. 2 illustrates the conformational changes undergone by a coronavirus spike protein prefusion with a host cell membrane.

FIG. 3 illustrates several configurations of up and down conformations assumed by the receptor binding domains of various coronavirus spike proteins.

FIG. 4 illustrates an experimental strategy for designing and screening variant coronavirus spike proteins.

FIG. 5 illustrates the conformation of a coronavirus spike protein prefusion with a host cell membrane and post-fusion with a host cell membrane and the location of three amino acid residues with respect to the SARS-CoV-2 spike protein sequence that are modified, in some aspects, to produce variant coronavirus spike proteins.

FIG. 6 illustrates the location of three amino acid residues with respect to the SARS-CoV-2 spike protein sequence that are modified, in some aspects, to produce variant coronavirus spike proteins and the effect of these modifications on spike protein expression and localization to the surface of a host cell membrane.

FIG. 7A-7B. (A) and (B) illustrate the location of one amino acid residue with respect to the SARS-CoV-2 spike protein sequence (A) and the location of the residue when it is modified (B). (A) and (B) demonstrate the effect of this modification on the conformation assumed by the receptor binding domains of the variant coronavirus spike protein.

FIG. 8 provides protein expression, ACE2 binding, and 3022 binding levels of the variant coronavirus spike proteins according to some aspects disclosed herein.

FIG. 9 Describes some of the amino acid residues that are modified, in some aspects, to produce variant coronavirus spike proteins. In some instances, these specific modifications are used in addition to other amino acid residue modifications, such as some of the combinations in FIG. 10.

FIG. 10 describes some of the amino acid residues that are modified, in some aspects, to produce variant coronavirus spike proteins and the effect of these modifications on protein expression, ACE2 binding, and 3022 binding by the variant coronavirus spike proteins. In some instances, the modifications are made in addition to the modifications described in FIG. 9 as indicated.

### DETAILED DESCRIPTION

### I. Examples of Definitions

Throughout this application, the term “about” is used according to its plain and ordinary meaning in the area of cell and molecular biology to indicate that a value includes the inherent variation or standard deviation of error for the measurement or quantitation method being employed to determine the value. For example, in some aspects, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the measurement or quantitation.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The phrase “essentially all” is defined as “at least 95%”; if essentially all members of a group have a certain property, then at least 95% of members of the group have that property. In some instances, essentially all means equal to any one of, at least any one of, or between any two of 95, 96, 97, 98, 99, or 100% of members of the group have that property.

The compositions and methods for their use can “comprise,” “consist essentially of,” or “consist of” any of the ingredients or steps disclosed throughout the specification. Throughout this specification, unless the context requires otherwise, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and will be understood to imply the inclusion of a stated step or element or group of steps or elements but not the exclusion of any other step or element or group of steps or elements. It is contemplated that aspects described herein in the context of the term “comprising” may also be implemented in the context of the term “consisting of” or “consisting essentially of” Compositions and methods “consisting essentially of” any of the ingredients or steps disclosed limits the scope of the claim to the specified materials or steps which do not materially affect the basic and novel characteristic of the claimed disclosure. The words “consisting of” (and any form of consisting of, such as “consist of” and “consists of”) means including, and limited to, whatever follows the phrase “consisting of.” Thus, the phrase “consisting of” indicates that the listed elements are required or mandatory, and that no other elements may be present.

Reference throughout this specification to “one aspect,” “an aspect,” “a particular aspect,” “a related aspect,” “a certain aspect,” “an additional aspect,” or “a further aspect” or combinations thereof means that a particular feature, structure or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

The terms “inhibiting” or “reducing” or any variation of these terms includes any measurable decrease or complete inhibition to achieve a desired result. The terms “improve,” “promote,” or “increase” or any variation of these terms includes any measurable increase to achieve a desired result or production of a protein or molecule.

As used herein, the terms “reference,” “standard,” or “control” describe a value relative to which a comparison is performed. For example, an agent, subject, population, sample, or value of interest is compared with a reference, standard, or control agent, subject, population, sample, or value of interest. A reference, standard, or control may be tested and/or determined substantially simultaneously and/or with the testing or determination of interest for an agent, subject, population, sample, or value of interest and/or may be determined or characterized under comparable conditions or circumstances to the agent, subject, population, sample, or value of interest under assessment.

The term “DNA,” as used herein, means a nucleic acid molecule that includes deoxyribonucleotide residues (such as containing the nucleotide base(s) adenine (A), cytosine (C), guanine (G) and/or thymine (T)). For example, DNA can contain all, or a majority of, deoxyribonucleotide residues. As used herein, the term “deoxyribonucleotide” means a nucleotide lacking a hydroxyl group at the 2′ position of a β-D-ribofuranosyl group. Without any limitation, DNA can encompass double stranded DNA, antisense DNA, single stranded DNA, isolated DNA, synthetic DNA, DNA that is recombinantly produced, and modified DNA.

As used herein, a “protein,” “polypeptide,” or “peptide” refers to a molecule comprising at least two amino acid residues. As used herein, the term “wild-type” or “native” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity. Where a protein is specifically mentioned herein, it is in general a reference to a native (wild-type) or recombinant (modified) protein. The protein may be isolated directly from the organism of which it is native, produced by recombinant DNA/exogenous expression methods, produced by solid-phase peptide synthesis (SPPS), or other in vitro methods. In particular aspects, there are isolated nucleic acid segments and recombinant vectors incorporating nucleic acid sequences that encode a polypeptide (e.g., an antigen or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that can be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state and/or that is altered or removed from the natural state through human intervention. For example, a DNA naturally present in a living animal is not “isolated,” but a synthetic DNA, or a DNA partially or completely separated from the coexisting materials of its natural state is “isolated.” An isolated nucleic acid can exist in substantially purified form, or can exist in a non-native environment such as, for example, a cell into which the nucleic acid has been delivered.

An immune response refers to a humoral response, a cellular response, or both a humoral and cellular response in an organism. An immune response can be measured by assays that include, but are not limited to, assays measuring the presence or amount of antibodies that specifically recognize a protein or cell surface protein, assays measuring T-cell activation or proliferation, and/or assays that measure modulation in terms of activity or expression of one or more cytokines.

### II. Viruses

As contemplated herein, without any limitations, the compositions and methods herein can be used as a modality to treat and/or prevent and/or reduce the severity of or medical/health risks of a number of diseases and/or conditions in mammals, including coronavirus infection in humans. Methods described herein comprise administration of the compositions described herein to a mammal, such as a human. For example, in one aspect, such methods of use for the compositions herein include a variant coronavirus spike protein or peptide vaccine or a variant coronavirus spike protein or peptide-coding nucleic acid vaccine to induce robust neutralizing antibodies and accompanying/concomitant T-cell response to achieve protective immunization with preferably minimal vaccine doses.

For example, such nucleic acid can be used to encode at least one antigen intended to generate an immune response in a mammal. These immunogenic polypeptide constructs are peptide or protein antigens derived from a pathogen associated with infectious disease, including viruses, bacteria, parasites, and fungi. The immunogenic polypeptide constructs can comprise one or more isolated immunogenic polypeptides comprising a variant coronavirus spike protein that is a variant of a native coronavirus spike protein or fragment thereof that elicit an immune response. In specific aspects, the immunogenic polypeptide constructs are variant coronavirus spike protein constructs, and the variant coronavirus spike protein constructs can comprise one or more immunogenic peptide sequences that elicit an immune response.

Conditions and/or diseases that can be treated and/or prevented with such nucleic acid and/or peptide or polypeptide compositions include, but are not limited to, those caused and/or impacted by viral infection. Such viruses include, but are not limited to, coronaviruses (such as a severe acute respiratory syndrome virus (SARS)—e.g. SARS-CoV-2, or a Middle East Respiratory Syndrome (MERS) virus).

Variant coronavirus spike protein constructs can be based on anyspike protein sequence from any Coronaviridae family virus. Coronaviridae is a family of enveloped, positive-sense, single-stranded RNA viruses. Coronavirus is the common name for Coronaviridae and Orthocoronavirinae (also referred to as Coronavirinae). The family Coronaviridae is organized in 2 sub-families, 5 genera, 23 sub-genera and approximately 40 species. They are enveloped viruses having a positive-sense single-stranded RNA genome and a nucleocapsid having helical symmetry.

Several coronaviruses utilize animals as their primary hosts and have also evolved to infect humans. There are four main sub-groupings of coronaviruses, known as alpha, beta, gamma, and delta; seven coronaviruses can infect people. The four most common coronaviruses utilize humans as their natural host and include: 229E (alpha coronavirus); NL63 (alpha coronavirus); OC43 (beta coronavirus); HKU1 (beta coronavirus). Three other human coronaviruses are: MERS-CoV (the beta coronavirus that causes MERS); SARS-CoV (the beta coronavirus that causes SARS); and SARS-CoV-2 (the novel coronavirus that causes coronavirus disease 2019, or COVID-19).

Coronaviruses have characteristic club-shaped spikes that project from their surface, which in electron micrographs create an image reminiscent of the solar corona, from which their name derives. The average diameter of the virus particles is around 120 nm (0.12 μm). The diameter of the envelope is ˜80 nm (0.08 m) and the spikes are ˜20 nm (0.02 μm) long. Beneath the spiked exterior of the virus is a round core shrouded in a viral envelope. The core contains genetic material that the virus can inject into cells to infect them.

The viral envelope consists of a lipid bilayer where the membrane (M), envelope (E), and spike (S) structural proteins are anchored. Inside the envelope, there is the nucleocapsid of helical symmetry which is formed from multiple copies of the nucleocapsid (N) protein, which are bound to the positive-sense single-stranded RNA genome in a continuous beads-on-a-string type conformation. The genome size of coronaviruses ranges from approximately 26 to 32 kilobases. The genome organization for a coronavirus is 5′-leader-UTR-replicase/transcriptase-spike (S)-envelope (E)-membrane (M)-nucleocapsid (N)-3′UTR-poly (A) tail. The open reading frames 1a and 1b, which occupy the first two-thirds of the genome, encode the replicase/transcriptase polyprotein. The replicase/transcriptase polyprotein self cleaves to form nonstructural proteins. The later reading frames encode the four major structural proteins: spike, envelope, membrane, and nucleocapsid. Interspersed between these reading frames are the reading frames for the accessory proteins. The number of accessory proteins and their function is unique depending on the specific coronavirus.

The lipid bilayer envelope, membrane proteins, and nucleocapsid protect the virus when it is outside the host cell. The spike proteins extend from within the core to the viral surface and allow the virus to recognize and bind specific cells in the body. When the spike engages a receptor on a host cell, a cascade is triggered, resulting in the merger of the virus with the cell which allows the virus to release its genetic material and overtake the cell's processes to produce new viruses.

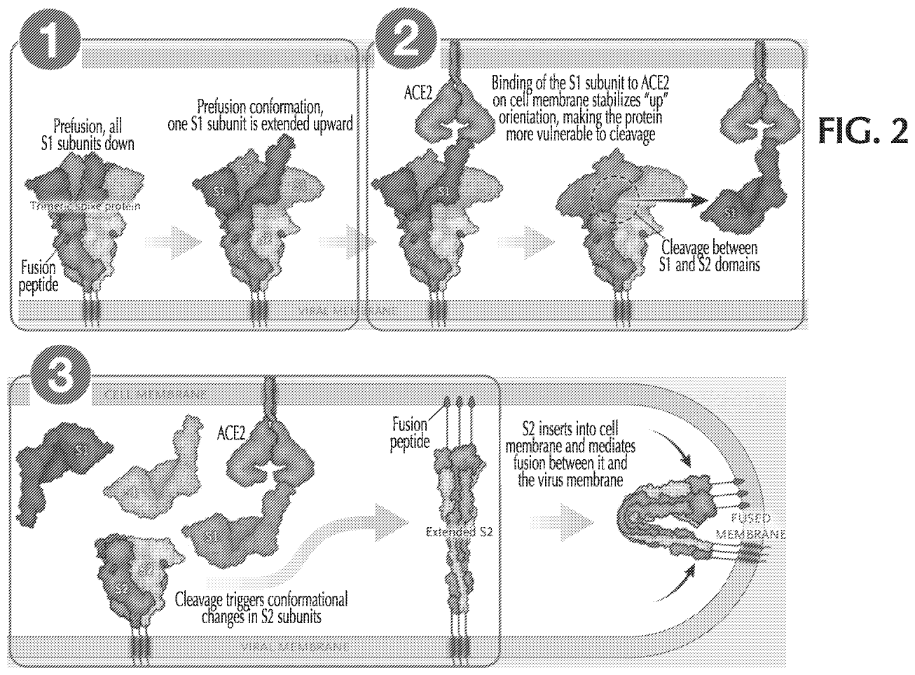

Infection begins when the viral spike (S) glycoprotein attaches to its complementary host cell receptor. After attachment, a protease of the host cell (e.g., ACE2) cleaves and activates the receptor-attached spike protein. Depending on the host cell protease available, cleavage and activation allows the virus to enter the host cell by endocytosis or direct fusion of the viral envelop with the host membrane (FIG. 2).

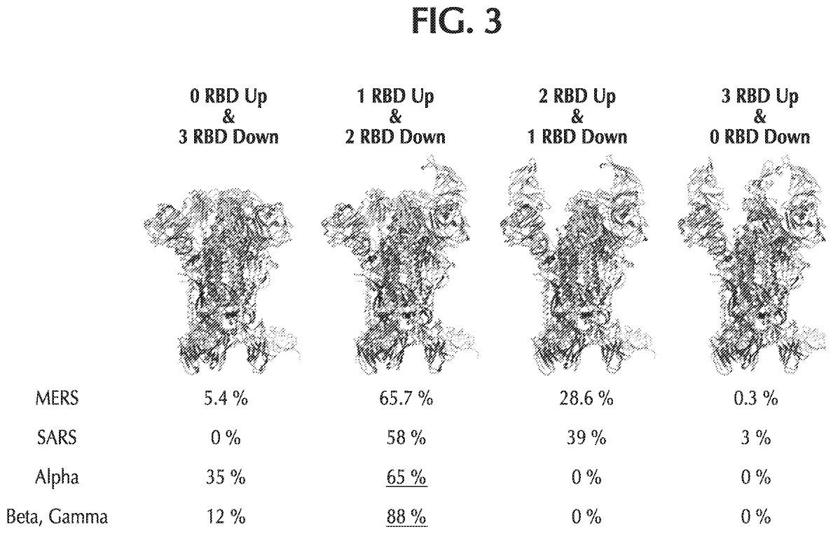

Binding of the S1 subunit of the S protein to the host cell receptor stabilizes the S protein in an “up” conformation, making the protein more vulnerable to cleavage by the host cell protease because the receptor binding site is exposed when the S protein is in the RBD-up conformation. Additionally, neutralization-sensitive epitopes are exposed when the RBD is in the up conformation, and thus, in some aspects of the variant coronavirus spike proteins disclosed herein, modifications are made to promote adoption of the RBD-up conformation or to inhibit adoption of the RBD-down conformation. This conformation of the S protein is illustrated by the crystal structures of a coronavirus shown in FIG. 2 and FIG. 3. Cleavage occurs between the S1 and S2 subunits of the S protein, and cleavage triggers conformational changes by the S2 subunit to allow insertion of the S2 subunit into the host cell membrane and mediation of fusion between the viral and host cell membranes.

On entry into the host cell, the virus particle is uncoated, and its genome enters the cell cytoplasm. The coronavirus RNA genome has a 5′ methylated cap and a 3′ polyadenylated tail, which allows the RNA to attach to the host cell's ribosome for translation. The host ribosome translates the initial overlapping open reading frame of the virus genome and forms a long polyprotein. The polyprotein has its own proteases which cleave the polyprotein into multiple nonstructural proteins.

Viral entry is followed by replication of the virus. A number of the nonstructural proteins coalesce to form a multi-protein replicase-transcriptase complex (RTC). The main replicase-transcriptase protein is the RNA-dependent RNA polymerase (RdRp). It is directly involved in the replication and transcription of RNA from an RNA strand. The other nonstructural proteins in the complex assist in the replication and transcription process. The exoribonuclease nonstructural protein, for instance, provides extra fidelity to replication by providing a proofreading function which the RNA-dependent RNA polymerase lacks. One of the main functions of the complex is to replicate the viral genome. RdRp directly mediates the synthesis of negative-sense genomic RNA from the positive-sense genomic RNA. This is followed by the replication of positive-sense genomic RNA from the negative-sense genomic RNA. The other important function of the complex is to transcribe the viral genome. RdRp directly mediates the synthesis of negative-sense subgenomic RNA molecules from the positive-sense genomic RNA. This is followed by the transcription of these negative-sense subgenomic RNA molecules to their corresponding positive-sense mRNAs.

The replicated positive-sense genomic RNA becomes the genome of the progeny viruses. The mRNAs are gene transcripts of the last third of the virus genome after the initial overlapping reading frame. These mRNAs are translated by the host's ribosomes into the structural proteins and a number of accessory proteins. RNA translation occurs inside the endoplasmic reticulum. The viral structural proteins S, E, and M move along the secretory pathway into the Golgi intermediate compartment. There, the M proteins direct most protein-protein interactions required for assembly of viruses following its binding to the nucleocapsid. Progeny viruses are then released from the host cell by exocytosis through secretory vesicles.

The interaction of the coronavirus spike protein with its complement host cell receptor is central in determining the tissue tropism, infectivity, and species range of the virus. Coronaviruses mainly target epithelial cell receptors. They can be transmitted by aerosol, fomite, or fecal-oral routes, for example. Human coronaviruses infect the epithelial cells of the respiratory tract. For example, human coronaviruses can infect, via an aerosol route, human epithelial cells of the lungs by binding of the spike protein receptor binding domain (RBD) to an angiotensin-converting enzyme 2 (ACE2) receptor on the cell surface.

The WHO has reported that the two groups most at risk of experiencing severe illness due to a coronavirus infection are adults aged 65 years or older and people who have other underlying health conditions including chronic lung disease, serious heart conditions, severe obesity, a compromised immune system, or diabetes. In humans, coronaviruses typically cause a respiratory infection with mild to severe flu-like symptoms, but the exact symptoms vary depending on the type of coronavirus. The four common human coronaviruses can cause people to develop a runny nose, headache, cough, sore throat and fever. In a subset of individuals, including those with cardiopulmonary disease or a weakened immune system, the viral infection can progress to a more severe lower-respiratory infection such as pneumonia or bronchitis. In comparison, severe MERS and SARS infections often progress to pneumonia. Other symptoms of MERS include fever, coughing, and shortness of breath, while SARS can cause fever, chills and body aches.

SARS-CoV-2 causes symptoms similar to those of other coronaviruses, triggering fever, cough, and shortness of breath in most patients. Rarer symptoms include dizziness, tiredness, aches, chills, sore throat, loss of smell, loss of taste, headache, nausea, vomiting, and diarrhea. Emergency signs or symptoms can include trouble breathing, persistent chest pain or pressure, new confusion, and/or blue lips or face. Complications of SARS-CoV-2 infections can include pneumonia, organ failure, respiratory failure, blood clots, heart conditions such as cardiomyopathies, acute kidney injury, and/or further viral and bacterial infections.

The present disclosure encompasses treatment or prevention of a disease or condition caused by infection of any virus in the Coronaviridae family. In particular aspects, methods and compositions treat or prevent COVID-19 or reduce the symptoms or severity of COVID-19, which is caused by infection from SARS-CoV-2. In certain aspects, the disclosure encompasses treatment or prevention of infection of any virus in the subfamily Coronavirinae and including the four genera, Alpha-, Beta-, Gamma-, and Deltacoronavirus. In specific aspects, the disclosure encompasses treatment or prevention of infection of any virus in the genus of Betacoronavirus, including the subgenus Sarbecovirus and the species severe acute respiratory syndrome-related coronavirus; the subgenus Embecovirus and the species human coronavirus HKU1; and the species Betacoronavirus 1. In specific aspects, the disclosure encompasses treatment or prevention of infection of any virus in the species of severe acute respiratory syndrome-related coronavirus, including the strains severe acute respiratory syndrome coronavirus (SARS-CoV) and severe acute respiratory syndrome coronavirus 2 (SARS-CoV-2, the virus that causes COVID-19). The disclosure encompasses treatment or prevention of infection any isolate, strain, type (including Type A, Type B and Type C; Forster et al., 2020, PNAS, available on the World Wide Web at doi.org/10.1073/pnas.2004999117), cluster, or sub-cluster of the species of severe acute respiratory syndrome-related coronavirus, including at least SARS-CoV-2. In specific aspects, the virus has a genome length between 29000 to 30000, between 29100 and 29900, between 29200 and 29900, between 29300 and 29900, between 29400 and 29900, between 29500 and 29900, between 29600 and 29900, between 29700 and 29900, between 29800 and 29900, or between 29780 and 29900 base pairs in length.

Examples of specific SARS-CoV-2 viruses include the following listed in the NCBI GenBank® Database, and these GenBank® Accession sequences are incorporated by reference herein in their entirety: (a) LC534419 and LC534418 and LC528233 and LC529905 (examples of different strains from Japan); (b) MT281577 and MT226610 and NC_045512 and MN996531 and MN908947 (examples of different strains from China); (c) MT281530 (Iran); (d) MT126808 (Brazil); (e) MT020781 (Finland); (f) MT093571 (Sweden); (g) MT263074 (Peru); (h) MT292582 and MT292581 and MT292580 and MT292579 (examples of different strains from Spain); (i) examples from the United States, such as MT276331 (TX); MT276330 (FL); MT276328 (OR) MT276327 (GA); MT276325 (WA); MT276324 (CA); MT276323 (RI); MT188341 (MN); and (j) MT276598 (Israel). In particular aspects, the disclosure encompasses treatment or prevention of infection of any of these or similar viruses, including viruses whose genome has at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. In particular aspects, the disclosure encompasses treatment or prevention of infection of any of these or similar viruses, including viruses whose genome has its entire sequence that is greater than 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. As one specific example, the present disclosure includes methods of treatment or prevention of infection of a virus having a genome sequence of GenBank® Accession No. NC_045512 and any virus having a genome sequence with at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to said sequence.

### III. Nucleic Acids

In certain aspects, an immunogenic polypeptide construct, e.g., an isolated immunogenic polypeptide comprising a variant coronavirus spike protein that is a variant of a native coronavirus spike protein or fragment thereof, is intended to generate an immune response and is encoded by a nucleic acid. The nucleic acids encoding the immunogenic polypeptide constructs can comprise one or more nucleotide sequences corresponding to one or more immunogenic peptide sequences that elicit an immune response.

Nucleic acid sequences can exist in a variety of instances such as: isolated segments; recombinant vectors of incorporated sequences or recombinant polynucleotides encoding polypeptides, such as antigens or one or both chains of an antibody, or a fragment, derivative, mutein, or variant thereof, polynucleotides sufficient for use as hybridization probes, PCR primers, or sequencing primers for identifying, analyzing, mutating or amplifying a polynucleotide; anti-sense nucleic acids for inhibiting expression of a polynucleotide; mRNA; saRNA; and complementary sequences of the foregoing described herein. Nucleic acids may encode an antigen or epitope to which antibodies may bind. Nucleic acids encoding fusion proteins that include antigens or epitopes are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that can be recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar polypeptide.

In certain aspects, there are polynucleotide variants having substantial identity to the sequences disclosed herein; those comprising equal to any one of, at least any one of, at most any one of, or between any two of 70%, 75%, 80%, 85%, 90%, 95%, 96%, 97%, 98%, or 99% or higher sequence identity, compared to a polynucleotide sequence provided herein using the methods described herein (e.g., BLAST analysis using standard parameters). In certain aspects, the isolated polynucleotide will comprise a nucleotide sequence encoding a polypeptide that has at least 90% identity to an amino acid sequence described herein, over the entire length of the sequence; or a nucleotide sequence complementary to said isolated polynucleotide. In some aspects, the isolated polynucleotide will comprise a nucleotide sequence encoding a polypeptide that has at least 95% identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, equal to any one of, at least any one of, at most any one of, or between any two of 5, 10, 15, 20, 25, 30, 35, 40, 45, 50, 75, 100, 125, 175, 200, 250, 300, 350, 400, 450, 500, 750, 1000, 1500, 3000, 5000, 6000, 7000, 8000, 9000, 10000, 11000, 12000, 13000, 14000, 15000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic and/or prophylactic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In some aspects, one or more isolated immunogenic polypeptides comprising a variant coronavirus spike protein that is a variant of a native coronavirus spike protein or fragment thereof are encoded by a nucleic acid and elicit an immune response. The immune response may be against the immunogenic variant coronavirus spike protein constructs and/or immunogenic native coronavirus spike protein sequences.

The immunogenic variant coronavirus spike protein constructs may comprise equal to any one of, at least any one of, at most any one of, or between any two of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, or more contiguous nucleotides that are equal to any one of, at least any one of, at most any one of, or between any two of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with a nucleic acid encoding a native coronavirus spike protein or a cDNA encoding a native coronavirus spike host protein.

The nucleic acids of the disclosure encoding the isolated immunogenic polypeptide comprising a variant coronavirus spike protein that is a variant of a native coronavirus spike protein or fragment thereof may include equal to any one of, at least any one of, at most any one of, or between any two of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54 mutations in the sequence encoding the immunogenic variant coronavirus spike protein constructs compared to nucleic acids encoding unmodified, native immunogenic coronavirus spike protein constructs. The portions of the nucleic acids of the disclosure encoding the immunogenic variant coronavirus spike protein constructs may equal to any one of, at least any one of, at most any one of, or between any two of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% (or any derivable range therein) similar, identical, or homologous with nucleic acids encoding unmodified, native immunogenic coronavirus spike protein constructs.

### A. Substitution

Changes can be introduced by substitution of a nucleic acid, thereby leading to changes in the amino acid sequence of a polypeptide (e.g., an antigen or antibody or antibody derivative) that it encodes. Substitutions can be introduced using any technique known in the art. In one aspect, one or more particular amino acid residues are changed using, for example, a site-directed mutagenesis protocol.

In some instances, substitutions can be introduced into a nucleic acid without significantly altering the biological activity of a polypeptide that it encodes. For example, one can make nucleotide substitutions leading to amino acid substitutions at non-essential amino acid residues. Alternatively, one or more substitutions can be introduced into a nucleic acid that selectively changes the biological activity of a polypeptide that it encodes. See, e.g., Romain Studer et al., Biochem. J. 449:581-594 (2013). For example, the substitutions can quantitatively or qualitatively change the biological activity. Examples of quantitative changes include increasing, reducing or eliminating the activity. Examples of qualitative changes include altering the antigen specificity of an antibody.

### IV. Polypeptides

The isolated immunogenic polypeptide constructs can comprise one or more isolated immunogenic polypeptides comprising a variant coronavirus spike protein that is a variant of a native coronavirus spike protein or fragment thereof that elicit an immune response. Thus, in certain aspects, isolated immunogenic polypeptide constructs are peptide or protein antigens derived from a pathogen associated with infectious disease, including coronaviruses. In certain aspects, the immunogenic polypeptide constructs are peptide or protein antigens derived from the spike protein of a coronavirus.

In certain aspects, peptides or proteins can exist in a variety of instances such as: isolated polypeptides or recombinant polypeptides, or a fragment, functional derivatives, muteins, or variants thereof, peptides or proteins sufficient for use as hybridization probes, peptides or proteins for inhibiting expression of a polynucleotide, and complementary amino acid sequences of the foregoing described herein. Peptides or proteins may be an epitope to which antibodies may bind. The peptides or proteins can comprise RNA and/or DNA nucleotides (e.g., peptide nucleic acids).

In certain aspects the size of a protein or peptide or derivative of a corresponding amino sequence described or referenced herein can be, for example, equal to any one of, at least any one of, at most any one of, or between any two of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500, 3000, 5000, 6000, 7000, 8000, 9000, 10000, 11000, 12000, 13000, 14000, 15000 amino acid residues or greater. It is contemplated that proteins or peptides may be mutated by truncation, rendering them shorter than their corresponding native or wild-type form, also, they might be altered by fusing or conjugating a heterologous protein or peptide sequence with a particular function (e.g., for targeting or localization, for enhanced immunogenicity, for purification purposes, etc.). As used herein, the term “domain” refers to any distinct functional or structural unit of a protein or peptide, and generally refers to a sequence of amino acids with a structure or function recognizable by one skilled in the art.

In some aspects, the one or more isolated immunogenic polypeptides comprising a variant coronavirus spike protein that is a variant of a native coronavirus spike protein or fragment thereof comprise at least a RBD. The RBD may or may not be modified with respect to the RBD of the native coronavirus spike protein. In some aspects, the RBD is modified with respect to the RBD of the native coronavirus spike protein.

In some aspects, the one or more isolated immunogenic polypeptides comprising a variant coronavirus spike protein that is a variant of a native coronavirus spike protein or fragment thereof comprise a leader sequence. In some aspects, the leader sequence has an amino acid sequence that has equal to any one of, at least any one of, at most any one of, or between any two of 70%, 75%, 80%, 85%, 90%, 95%, 96%, 97%, 98%, or 99% or higher sequence identity, compared to the leader sequence of the native coronavirus spike protein sequence. In some aspects, the leader sequence has an amino acid sequence that is at least 80% identical to the leader sequence of the native coronavirus spike protein sequence. In some aspects, inclusion of a leader sequence as part of the isolated immunogenic polypeptide sequence inhibits disulfide scrambling.

In some aspects, the one or more isolated immunogenic polypeptides comprising a variant coronavirus spike protein that is a variant of a native coronavirus spike protein or fragment thereof comprise a transmembrane sequence. In some aspects, the transmembrane sequence has an amino acid sequence that has equal to any one of, at least any one of, at most any one of, or between any two of 70%, 75%, 80%, 85%, 90%, 95%, 96%, 97%, 98%, or 99% or higher sequence identity, compared to the transmembrane sequence of the native coronavirus spike protein sequence. In some aspects, the transmembrane sequence has an amino acid sequence that is at least 80% identical to the transmembrane sequence of the native coronavirus spike protein sequence. In some aspects, inclusion of a transmembrane sequence as part of the isolated immunogenic polypeptide sequence extends the half-life of the isolated immunogenic polypeptide.

In some aspects, the one or more isolated immunogenic polypeptides comprising a variant coronavirus spike protein that is a variant of a native coronavirus spike protein or fragment thereof does not comprise an endoplasmic reticulum (ER) signal sequence. In some aspects, exclusion of an ER signal sequence improves localization of the variant coronavirus spike protein to the host cell membrane.

In some aspects, the one or more isolated immunogenic polypeptides comprising a variant coronavirus spike protein that is a variant of a native coronavirus spike protein or fragment thereof comprise a trimerization domain. In some aspects, the trimerization domain is a foldon trimerization domain. In some aspects, the trimerization domain sequence has an amino acid sequence that has equal to any one of, at least any one of, at most any one of, or between any two of 70%, 75%, 80%, 85%, 90%, 95%, 96%, 97%, 98%, or 99% or higher sequence identity, compared to the trimerization domain sequence of the foldon trimerization domain of T4 fibritin. In some aspects, the trimerization domain sequence has an amino acid sequence that is at least 80% identical to the trimerization domain sequence of the foldon trimerization domain of T4 fibritin.

In some aspects, one or more isolated immunogenic polypeptides comprising a variant coronavirus spike protein that is a variant of a native coronavirus spike protein or fragment thereof are encoded by a nucleic acid and elicit an immune response. The immune response may be against the immunogenic variant coronavirus spike protein constructs and/or a native coronavirus spike protein. The immunogenic variant coronavirus spike protein constructs and the native coronavirus spike protein may be equal to any one of, at least any one of, at most any one of, or between any two of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous.

In some aspects, the variant coronavirus spike protein constructs may comprise equal to any one of, at least any one of, at most any one of, or between any two of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, or more contiguous amino acids that are equal to any one of, at least any one of, at most any one of, or between any two of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with a native coronavirus spike protein.

The immunogenic variant coronavirus spike protein constructs thereof may include equal to any one of, at least any one of, at most any one of, or between any two of 1, 2, 3, 4, 5, 6, 7, 8, 9, 10, 11, 12, 13, 14, 15, 16, 17, or 18 amino acid substitutions as disclosed herein compared to native, unmodified immunogenic polypeptide constructs. The immunogenic variant coronavirus spike protein constructs may equal to any one of, at least any one of, at most any one of, or between any two of 1%, 2%, 3%, 4%, 5%, 6%, 7%, 8%, 9%, 10%, 11%, 12%, 130%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% (or any derivable range therein) similar, identical, or homologous with native, unmodified immunogenic polypeptide constructs.

Nucleotide as well as protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov) and The Universal Protein Resource (UniProt; on the World Wide Web at uniprot.org). The coding regions for these genes may be amplified and/or expressed using the techniques disclosed herein or as would be known to those of ordinary skill in the art.

### V. Coronavirus Spike Protein Modifications

The immunogenic native coronavirus spike protein constructs and/or nucleic acids encoding the immunogenic native coronavirus spike protein constructs of the present disclosure may be modified, such that they are substantially identical to the immunogenic variant coronavirus spike protein constructs and/or nucleic acids encoding the immunogenic variant coronavirus spike protein constructs comprised in immunogenic compositions described herein. In some aspects, the immunogenic variant coronavirus spike protein constructs and and/or nucleic acids encoding the immunogenic variant coronavirus spike protein continue to be bound by antibodies to elicit an immune response.

In particular aspects, it is the peptide sequences and/or nucleic acids encoding the peptide sequences of native coronavirus spike proteins that are modified. Thus, in some aspects, variant coronavirus spike proteins comprise equal to any one of, at least any one of, at most any one of, or between any two of 70%, 75%, 80%, 85%, 90%, 95%, 96%, 97%, 98%, or 99% or higher sequence identity, compared to the native coronavirus spike protein sequence. In some aspects, variant coronavirus spike proteins can comprise an amino acid sequence that is at least 70% identical to an amino acid sequence of the native coronavirus spike protein. In some aspects, variant coronavirus spike proteins can comprise an amino acid sequence that is at least 80% identical to an amino acid sequence of the native coronavirus spike protein. In some aspects, variant coronavirus spike proteins can comprise an amino acid sequence that is at least 90% identical to an amino acid sequence of the native coronavirus spike protein.

Polypeptide and/or nucleic acid sequences are “substantially identical” when optimally aligned using such programs as Clustal Omega, IGBLAST, GAP or BESTFIT using default gap weights, they share at least 70% identity, at least 80% sequence identity, at least 90% sequence identity, at least 95% sequence identity, at least 96% sequence identity, at least 97% sequence identity, at least 98% sequence identity, or at least 99% sequence identity or any range therein.

The immunogenic variant coronavirus spike protein constructs and/or nucleic acids encoding the immunogenic variant coronavirus spike protein constructs of the disclosure may include equal to any one of, at least any one of, at most any one of, or between any two of 1, 2, 3, 4, 5, 6, 7, 8, 9, 10, 11, 12, 13, 14, 15, 16, 17, or 18 amino acid substitutions as disclosed herein and/or be equal to any one of, at least any one of, at most any one of, or between any two of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% similar, identical, or homologous with equal to any one of, at least any one of, at most any one of, or between any two of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, or more contiguous amino acids of native, unmodified immunogenic coronavirus spike protein constructs and/or nucleic acids encoding the native, unmodified immunogenic coronavirus spike protein constructs of the present disclosure and/or of homologous peptides or proteins.

In some aspects, the immunogenic variant coronavirus spike protein constructs and/or nucleic acids encoding the immunogenic variant coronavirus spike protein constructs may comprise equal to any one of, at least any one of, at most any one of, or between any two of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, or more contiguous amino acids that are equal to any one of, at least any one of, at most any one of, or between any two of 1%, 2%, 3%, 4%, 5%, 6%, 7%, 8%, 9%, 10%, 11%, 12%, 130%, 14%, 15%, 16%, 17%, 18%, 19%, 20%, 210%, 22%, 230%, 24%, 250%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with native, unmodified immunogenic coronavirus spike protein constructs and/or nucleic acids encoding the native, unmodified immunogenic coronavirus spike protein constructs of the present disclosure and/or of homologous peptides or proteins.

As modifications and/or changes may be made in the sequence and/or structure of polynucleotides and/or proteins according to the present disclosure, while obtaining molecules having similar or improved characteristics (e.g., maintenance of antibody binding and immune response and attenuation of cross-reactivity with endogenously-expressed host proteins), such biologically functional equivalents of the immunogenic polypeptide constructs and/or nucleic acids encoding the immunogenic polypeptide constructs are also encompassed within the present invention.

The immunogenic variant coronavirus spike protein constructs and/or nucleic acids encoding the immunogenic variant coronavirus spike protein constructs may comprise a polynucleotide that has been engineered to contain distinct sequences while at the same time retaining the capacity to encode the “wild-type” or standard protein or peptide or “modified” or “variant” protein or peptide. This can be accomplished to the degeneracy of the genetic code, i.e., the presence of multiple codons, which encode for the same amino acids. The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids. In one example, one of skill in the art may wish to introduce a mutation into a polynucleotide to reduce cross-reactivity of the protein encoded by the polynucleotide with endogenously-expressed host proteins while not disturbing the ability of that polynucleotide to encode a protein that is bound by an antibody and that elicits an immune response.

In terms of functional equivalents, it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity. Biologically functional equivalents are thus defined herein as those proteins (and polynucleotides) having substitutions or mutations in selected amino acids (or codons) that retain the ability to be bound by an antibody and elicit an immune response and/or proteins (and polynucleotides) having substitutions or mutations in selected amino acids (or codons).

In one example, a polynucleotide may be (and encode) a biological functional equivalent with significant changes. Certain amino acids may be substituted for other amino acids in a protein structure without appreciable loss of interactive binding capacity with structures such as, for example, antigen-binding regions of antibodies, binding sites on substrate molecules, receptors, and such like.

### A. Coronavirus Spike Proteins

The following Table 1 includes the amino acid sequences of the spike proteins of the seven coronaviruses known to infect humans. Amino acid sequences were obtained from the UniProt database, accessible via the World Wide Web at uniprot.org, or the GenBank database, accessible via the World Wide Web at ncbi.nlm.nih.gov, and the UniProt or GenBank database accession numbers of each spike protein sequence are included in the Table 1. These amino acid sequences correspond to the amino acid sequences of native coronavirus spike proteins. In some aspects, the amino acid sequences of native coronavirus spike proteins may be modified, as described herein, to produce isolated immunogenic polypeptides comprising variant coronavirus spike proteins that are modifications of native coronavirus spike proteins or fragments thereof. For example, in some aspects, the amino acid sequences of native coronavirus spike proteins are substituted, as described herein, to produce isolated immunogenic polypeptides comprising variant coronavirus spike proteins that are modifications of native coronavirus spike proteins or fragments thereof.

Also included in the following Table 1 are the amino acid sequences of the spike proteins of SARS-CoV-2 variants, including the alpha, beta, gamma, delta, and omicron variants (including omicron BA1 and BA2). Like the amino acid sequences of native coronavirus spike proteins, the amino acid sequences of spike proteins of these SARS-CoV-2 variants may be modified at the corresponding position, as described herein, to produce isolated immunogenic polypeptides comprising variant coronavirus spike proteins that are modifications of the native variant coronavirus spike proteins or fragments thereof. For example, in some aspects, the amino acid sequences of spike proteins of these SARS-CoV-2 variants are substituted, as described herein, to produce isolated immunogenic polypeptides comprising variant coronavirus spike proteins that are modifications of variant coronavirus spike proteins or fragments thereof. Additional variants not specifically set forth below are also contemplated. For example, any variant coronavirus spike protein having 70%, 75%, 80%, 85%, 90%, 95%, 96%, 97%, 98%, or 99% or higher sequence identity with the native coronavirus spike protein sequence may be modified at the corresponding position, (e.g., substituted), as described herein, to produce isolated immunogenic polypeptides comprising variant coronavirus spike proteins that are modifications of native coronavirus spike proteins or fragments thereof.

TABLE 1

| | Accession | | |
|---|---|---|---|
| Virus | Number | Sequence | |
| MERS-CoV | K9N5Q8-1 | MIHSVFLLMFLLTPTESYVDVGPDSVKSACIEVDIQQTFFD | |
| | | KTWPRPIDVSKADGIIYPQGRTYSNITITYQGLFPYQGDHG | |
| | | DMYVYSAGHATGTTPQKLFVANYSQDVKQFANGFVVRIGAA | |
| | | ANSTGTVIISPSTSATIRKIYPAFMLGSSVGNFSDGKMGRF | |
| | | FNHTLVLLPDGCGTLLRAFYCILEPRSGNHCPAGNSYTSFA | |
| | | TYHTPATDCSDGNYNRNASLNSFKEYFNLRNCTFMYTYNIT | |
| | | EDEILEWFGITQTAQGVHLFSSRYVDLYGGNMFQFATLPVY | |
| | | DTIKYYSIIPHSIRSIQSDRKAWAAFYVYKLQPLTFLLDFS | |
| | | VDGYIRRAIDCGFNDLSQLHCSYESFDVESGVYSVSSFEAK | |
| | | PSGSVVEQAEGVECDFSPLLSGTPPQVYNFKRLVFTNCNYN | |
| | | LTKLLSLFSVNDFTCSQISPAAIASNCYSSLILDYFSYPLS | |
| | | MKSDLSVSSAGPISQFNYKQSFSNPTCLILATVPHNLTTIT | |
| | | KPLKYSYINKCSRFLSDDRTEVPQLVNANQYSPCVSIVPST | |
| | | VWEDGDYYRKQLSPLEGGGWLVASGSTVAMTEQLQMGFGIT | |
| | | VQYGTDTNSVCPKLEFANDTKIASQLGNCVEYSLYGVSGRG | |
| | | VFQNCTAVGVRQQRFVYDAYQNLVGYYSDDGNYYCLRACVS | |
| | | VPVSVIYDKETKTHATLFGSVACEHISSTMSQYSRSTRSML | |
| | | KRRDSTYGPLQTPVGCVLGLVNSSLFVEDCKLPLGQSLCAL | |
| | | PDTPSTLTPRSVRSVPGEMRLASIAFNHPIQVDQLNSSYFK | |
| | | LSIPTNFSFGVTQEYIQTTIQKVTVDCKQYVCNGFQKCEQL | |
| | | LREYGQFCSKINQALHGANLRQDDSVRNLFASVKSSQSSPI | |
| | | IPGFGGDFNLTLLEPVSISTGSRSARSAIEDLLFDKVTIAD | |
| | | PGYMQGYDDCMQQGPASARDLICAQYVAGYKVLPPLMDVNM | |
| | | EAAYTSSLLGSIAGVGWTAGLSSFAAIPFAQSIFYRLNGVG | |
| | | ITQQVLSENQKLIANKFNQALGAMQTGFTTTNEAFHKVQDA | |
| | | VNNNAQALSKLASELSNTFGAISASIGDIIQRLDVLEQDAQ | |
| | | IDRLINGRLTTLNAFVAQQLVRSESAALSAQLAKDKVNECV | |
| | | KAQSKRSGFCGQGTHIVSFVVNAPNGLYFMHVGYYPSNHIE | |
| | | VVSAYGLCDAANPTNCIAPVNGYFIKTNNTRIVDEWSYTGS | |
| | | SFYAPEPITSLNTKYVAPQVTYQNISTNLPPPLLGNSTGID | |
| | | FQDELDEFFKNVSTSIPNFGSLTQINTTLLDLTYEMLSLQQ | |
| | | VVKALNESYIDLKELGNYTYYNKWPWYIWLGFIAGLVALAL | |
| | | CVFFILCCTGCGTNCMGKLKCNRCCDRYEEYDLEPHKVHVH | |
| | | (SEQ ID NO: 2) | |
| NL63-CoV | Q6Q1S2-1 | MKLFLILLVLPLASCFFTCNSNANLSMLQLGVPDNSSTIVT | |
| | | GLLPTHWFCANQSTSVYSANGFFYIDVGNHRSAFALHTGYY | |
| | | DANQYYIYVTNEIGLNASVTLKICKFSRNTTFDFLSNASSS | |
| | | FDCIVNLLFTEQLGAPLGITISGETVRLHLYNVTRTFYVPA | |
| | | AYKLTKLSVKCYFNYSCVFSVVNATVTVNVTTHNGRVVNYT | |
| | | VCDDCNGYTDNIFSVQQDGRIPNGFPFNNWFLLTNGSTLVD | |
| | | GVSRLYQPLRLTCLWPVPGLKSSTGFVYFNATGSDVNCNGY | |
| | | QHNSVVDVMRYNLNFSANSLDNLKSGVIVFKTLQYDVLFYC | |
| | | SNSSSGVLDTTIPFGPSSQPYYCFINSTINTTHVSTFVGIL | |
| | | PPTVREIVVARTGQFYINGFKYFDLGFIEAVNFNVTTASAT | |
| | | DFWTVAFATFVDVLVNVSATNIQNLLYCDSPFEKLQCEHLQ | |
| | | FGLQDGFYSANFLDDNVLPETYVALPIYYQHTDINFTATAS | |
| | | FGGSCYVCKPHQVNISLNGNTSVCVRTSHFSIRYIYNRVKS | |
| | | GSPGDSSWHIYLKSGTCPFSFSKLNNFQKFKTICFSTVEVP | |
| | | GSCNFPLEATWHYTSYTIVGALYVTWSEGNSITGVPYPVSG | |
| | | IREFSNLVLNNCTKYNIYDYVGTGIIRSSNQSLAGGITYVS | |
| | | NSGNLLGFKNVSTGNIFIVTPCNQPDQVAVYQQSIIGAMTA | |
| | | VNESRYGLQNLLQLPNFYYVSNGGNNCTTAVMTYSNFGICA | |
| | | DGSLIPVRPRNSSDNGISAIITANLSIPSNWTTSVQVEYLQ | |
| | | ITSTPIVVDCATYVCNGNPRCKNLLKQYTSACKTIEDALRL | |
| | | SAHLETNDVSSMLTFDSNAFSLANVTSFGDYNLSSVLPQRN | |
| | | IRSSRIAGRSALEDLLFSKVVTSGLGTVDVDYKSCTKGLSI | |
| | | ADLACAQYYNGIMVLPGVADAERMAMYTGSLIGGMVLGGLT | |
| | | SAAAIPFSLALQARLNYVALQTDVLQENQKILAASFNKAIN | |
| | | NIVASFSSVNDAITQTAEAIHTVTIALNKIQDVVNQQGSAL | |
| | | NHLTSQLRHNFQAISNSIQAIYDRLDSIQADQQVDRLITGR | |
| | | LAALNAFVSQVLNKYTEVRGSRRLAQQKINECVKSQSNRYG | |
| | | FCGNGTHIFSIVNSAPDGLLFLHTVLLPTDYKNVKAWSGIC | |
| | | VDGIYGYVLRQPNLVLYSDNGVFRVTSRVMFQPRLPVLSDF | |
| | | VQIYNCNVTFVNISRVELHTVIPDYVDVNKTLQEFAQNLPK | |
| | | YVKPNFDLTPFNLTYLNLSSELKQLEAKTASLFQTTVELQG | |
| | | LIDQINSTYVDLKLLNRFENYIKWPWWVWLIISVVFVVLLS | |
| | | LLVFCCLSTGCCGCCNCLTSSMRGCCDCGSTKLPYYEFEKV | |
| | | HVQ (SEQ ID NO: 3) | |
| 229E-CoV | P15423-1 | MFVLLVAYALLHIAGCQTTNGLNTSYSVCNGCVGYSENVFA | |
| | | VESGGYIPSDFAFNNWFLLTNTSSVVDGVVRSFQPLLLNCL | |
| | | WSVSGLRFTTGFVYFNGTGRGDCKGFSSDVLSDVIRYNLNF | |
| | | EENLRRGTILFKTSYGVVVFYCTNNTLVSGDAHIPFGTVLG | |
| | | NFYCFVNTTIGNETTSAFVGALPKTVREFVISRTGHFYING | |
| | | YRYFTLGNVEAVNFNVTTAETTDFCTVALASYADVLVNVSQ | |
| | | TSIANIIYCNSVINRLRCDQLSFDVPDGFYSTSPIQSVELP | |
| | | VSIVSLPVYHKHTFIVLYVDFKPQSGGGKCFNCYPAGVNIT | |
| | | LANFNETKGPLCVDTSHFTTKYVAVYANVGRWSASINTGNC | |
| | | PFSFGKVNNFVKFGSVCFSLKDIPGGCAMPIVANWAYSKYY | |
| | | TIGSLYVSWSDGDGITGVPQPVEGVSSFMNVTLDKCTKYNI | |
| | | YDVSGVGVIRVSNDTFLNGITYTSTSGNLLGFKDVTKGTIY | |
| | | SITPCNPPDQLVVYQQAVVGAMLSENFTSYGFSNVVELPKF | |
| | | FYASNGTYNCTDAVLTYSSFGVCADGSIIAVQPRNVSYDSV | |
| | | SAIVTANLSIPSNWTTSVQVEYLQITSTPIVVDCSTYVCNG | |
| | | NVRCVELLKQYTSACKTIEDALRNSARLESADVSEMLTFDK | |
| | | KAFTLANVSSFGDYNLSSVIPSLPTSGSRVAGRSAIEDILF | |
| | | SKLVTSGLGTVDADYKKCTKGLSIADLACAQYYNGIMVLPG | |
| | | VADAERMAMYTGSLIGGIALGGLTSAVSIPFSLAIQARLNY | |
| | | VALQTDVLQENQKILAASFNKAMTNIVDAFTGVNDAITQTS | |
| | | QALQTVATALNKIQDVVNQQGNSLNHLTSQLRQNFQAISSS | |
| | | IQAIYDRLDTIQADQQVDRLITGRLAALNVFVSHTLTKYTE | |
| | | VRASRQLAQQKVNECVKSQSKRYGFCGNGTHIFSIVNAAPE | |
| | | GLVFLHTVLLPTQYKDVEAWSGLCVDGTNGYVLRQPNLALY | |
| | | KEGNYYRITSRIMFEPRIPTMADFVQIENCNVTFVNISRSE | |
| | | LQTIVPEYIDVNKTLQELSYKLPNYTVPDLVVEQYNQTILN | |
| | | LTSEISTLENKSAELNYTVQKLQTLIDNINSTLVDLKWLNR | |
| | | VETYIKWPWWVWLCISVVLIFVVSMLLLCCCSTGCCGFFSC | |
| | | FASSIRGCCESTKLPYYDVEKIHIQ (SEQ ID NO: 4) | |
| OC43-CoV | P36334-1 | MFLILLISLPTAFAVIGDLKCTSDNINDKDTGPPPISTDTV | |
| | | DVTNGLGTYYVLDRVYLNTTLFLNGYYPTSGSTYRNMALKG | |
| | | SVLLSRLWFKPPFLSDFINGIFAKVKNTKVIKDRVMYSEFP | |
| | | AITIGSTFVNTSYSVVVQPRTINSTQDGDNKLQGLLEVSVC | |
| | | QYNMCEYPQTICHPNLGNHRKELWHLDTGVVSCLYKRNFTY | |
| | | DVNADYLYFHFYQEGGTFYAYFTDTGVVTKFLFNVYLGMAL | |
| | | SHYYVMPLTCNSKLTLEYWVTPLTSRQYLLAFNQDGIIFNA | |
| | | EDCMSDFMSEIKCKTQSIAPPTGVYELNGYTVQPIADVYRR | |
| | | KPNLPNCNIEAWLNDKSVPSPLNWERKTFSNCNFNMSSLMS | |
| | | FIQADSFTCNNIDAAKIYGMCFSSITIDKFAIPNGRKVDLQ | |
| | | LGNLGYLQSFNYRIDTTATSCQLYYNLPAANVSVSRFNPST | |
| | | WNKRFGFIEDSVFKPRPAGVLTNHDVVYAQHCFKAPKNFCP | |
| | | CKLNGSCVGSGPGKNNGIGTCPAGTNYLTCDNLCTPDPITF | |
| | | TGTYKCPQTKSLVGIGEHCSGLAVKSDYCGGNSCTCRPQAF | |
| | | LGWSADSCLQGDKCNIFANFILHDVNSGLTCSTDLQKANTD | |
| | | IILGVCVNYDLYGILGQGIFVEVNATYYNSWQNLLYDSNGN | |
| | | LYGFRDYIINRTFMIRSCYSGRVSAAFHANSSEPALLFRNI | |
| | | KCNYVFNNSLTRQLQPINYFDSYLGCVVNAYNSTAISVQTC | |
| | | DLTVGSGYCVDYSKNRRSRGAITTGYRFTNFEPFTVNSVND | |
| | | SLEPVGGLYEIQIPSEFTIGNMVEFIQTSSPKVTIDCAAFV | |
| | | CGDYAACKSQLVEYGSFCDNINAILTEVNELLDTTQLQVAN | |
| | | SLMNGVTLSTKLKDGVNFNVDDINFSPVLGCLGSECSKASS | |
| | | RSAIEDLLFDKVKLSDVGFVEAYNNCTGGAEIRDLICVQSY | |
| | | KGIKVLPPLLSENQISGYTLAATSASLFPPWTAAAGVPFYL | |
| | | NVQYRINGLGVTMDVLSQNQKLIANAFNNALYAIQEGFDAT | |
| | | NSALVKIQAVVNANAEALNNLLQQLSNRFGAISASLQEILS | |
| | | RLDALEAEAQIDRLINGRLTALNAYVSQQLSDSTLVKFSAA | |
| | | QAMEKVNECVKSQSSRINFCGNGNHIISLVQNAPYGLYFIH | |
| | | FSYVPTKYVTARVSPGLCIAGDRGIAPKSGYFVNVNNTWMY | |
| | | TGSGYYYPEPITENNVVVMSTCAVNYTKAPYVMLNTSIPNL | |
| | | PDFKEELDQWFKNQTSVAPDLSLDYINVTFLDLQVEMNRLQ | |
| | | EAIKVLNQSYINLKDIGTYEYYVKWPWYVWLLICLAGVAML | |
| | | VLLFFICCCTGCGTSCFKKCGGCCDDYTGYQELVIKTSHDD | |
| | | (SEQ ID NO: 5) | |
| HKU1 | Q5MQD0-1 | MLLIIFILPTTLAVIGDFNCTNFAINDLNTTVPRISEYVVD | |
| | | VSYGLGTYYILDRVYLNTTILFTGYFPKSGANFRDLSLKGT | |
| | | TYLSTLWYQKPFLSDFNNGIFSRVKNTKLYVNKTLYSEFST | |
| | | IVIGSVFINNSYTIVVQPHNGVLEITACQYTMCEYPHTICK | |
| | | SKGSSRNESWHFDKSEPLCLFKKNFTYNVSTDWLYFHFYQE | |
| | | RGTFYAYYADSGMPTTFLFSLYLGTLLSHYYVLPLTCNAIS | |
| | | SNTDNETLQYWVTPLSKRQYLLKFDNRGVITNAVDCSSSFF | |
| | | SEIQCKTKSLLPNTGVYDLSGFTVKPVATVHRRIPDLPDCD | |
| | | IDKWLNNFNVPSPLNWERKIFSNCNFNLSTLLRLVHTDSFS | |
| | | CNNFDESKIYGSCFKSIVLDKFAIPNSRRSDLQLGSSGFLQ | |
| | | SSNYKIDTTSSSCQLYYSLPAINVTINNYNPSSWNRRYGFN | |
| | | NFNLSSHSVVYSRYCFSVNNTFCPCAKPSFASSCKSHKPPS | |
| | | ASCPIGTNYRSCESTTVLDHTDWCRCSCLPDPITAYDPRSC | |
| | | SQKKSLVGVGEHCAGFGVDEEKCGVLDGSYNVSCLCSTDAF | |
| | | LGWSYDTCVSNNRCNIFSNFILNGINSGTTCSNDLLQPNTE | |
| | | VFTDVCVDYDLYGITGQGIFKEVSAVYYNSWQNLLYDSNGN | |
| | | IIGFKDFVTNKTYNIFPCYAGRVSAAFHQNASSLALLYRNL | |
| | | KCSYVLNNISLTTQPYFDSYLGCVFNADNLTDYSVSSCALR | |
| | | MGSGFCVDYNSPSSSSSRRKRRSISASYRFVTFEPFNVSFV | |
| | | NDSIESVGGLYEIKIPTNFTIVGQEEFIQTNSPKVTIDCSL | |
| | | FVCSNYAACHDLLSEYGTFCDNINSILDEVNGLLDTTQLHV | |
| | | ADTLMQGVTLSSNLNTNLHFDVDNINFKSLVGCLGPHCGSS | |
| | | SRSFFEDLLFDKVKLSDVGFVEAYNNCTGGSEIRDLLCVQS | |
| | | FNGIKVLPPILSESQISGYTTAATVAAMFPPWSAAAGIPFS | |
| | | LNVQYRINGLGVTMDVLNKNQKLIATAFNNALLSIQNGFSA | |
| | | TNSALAKIQSVVNSNAQALNSLLQQLFNKFGAISSSLQEIL | |
| | | SRLDALEAQVQIDRLINGRLTALNAYVSQQLSDISLVKFGA | |
| | | ALAMEKVNECVKSQSPRINFCGNGNHILSLVQNAPYGLLFM | |
| | | HFSYKPISFKTVLVSPGLCISGDVGIAPKQGYFIKHNDHWM | |
| | | FTGSSYYYPEPISDKNVVFMNTCSVNFTKAPLVYLNHSVPK | |
| | | LSDFESELSHWFKNQTSIAPNLTLNLHTINATFLDLYYEMN | |
| | | LIQESIKSLNNSYINLKDIGTYEMYVKWPWYVWLLISFSFI | |
| | | IFLVLLFFICCCTGCGSACFSKCHNCCDEYGGHHDFVIKTS | |
| | | HDD (SEQ ID NO: 6) | |
| SARS-CoV | P59594-1 | MFIFLLFLTLTSGSDLDRCTTFDDVQAPNYTQHTSSMRGVY | |
| | | YPDEIFRSDTLYLTQDLFLPFYSNVTGFHTINHTFGNPVIP | |
| | | FKDGIYFAATEKSNVVRGWVFGSTMNNKSQSVIIINNSTNV | |
| | | VIRACNFELCDNPFFAVSKPMGTQTHTMIFDNAFNCTFEYI | |
| | | SDAFSLDVSEKSGNFKHLREFVFKNKDGFLYVYKGYQPIDV | |
| | | VRDLPSGFNTLKPIFKLPLGINITNFRAILTAFSPAQDIWG | |
| | | TSAAAYFVGYLKPTTFMLKYDENGTITDAVDCSQNPLAELK | |
| | | CSVKSFEIDKGIYQTSNFRVVPSGDVVRFPNITNLCPFGEV | |
| | | FNATKFPSVYAWERKKISNCVADYSVLYNSTFFSTFKCYGV | |
| | | SATKLNDLCFSNVYADSFVVKGDDVRQIAPGQTGVIADYNY | |
| | | KLPDDFMGCVLAWNTRNIDATSTGNYNYKYRYLRHGKLRPF | |
| | | ERDISNVPFSPDGKPCTPPALNCYWPLNDYGFYTTTGIGYQ | |
| | | PYRVVVLSFELLNAPATVCGPKLSTDLIKNQCVNFNFNGLT | |
| | | GTGVLTPSSKRFQPFQQFGRDVSDFTDSVRDPKTSEILDIS | |
| | | PCSFGGVSVITPGTNASSEVAVLYQDVNCTDVSTAIHADQL | |
| | | TPAWRIYSTGNNVFQTQAGCLIGAEHVDTSYECDIPIGAGI | |
| | | CASYHTVSLLRSTSQKSIVAYTMSLGADSSIAYSNNTIAIP | |
| | | TNFSISITTEVMPVSMAKTSVDCNMYICGDSTECANLLLQY | |
| | | GSFCTQLNRALSGIAAEQDRNTREVFAQVKQMYKTPTLKYF | |
| | | GGFNFSQILPDPLKPTKRSFIEDLLFNKVTLADAGFMKQYG | |
| | | ECLGDINARDLICAQKFNGLTVLPPLLTDDMIAAYTAALVS | |
| | | GTATAGWTFGAGAALQIPFAMQMAYRFNGIGVTQNVLYENQ | |
| | | KQIANQFNKAISQIQESLTTTSTALGKLQDVVNQNAQALNT | |
| | | LVKQLSSNFGAISSVLNDILSRLDKVEAEVQIDRLITGRLQ | |
| | | SLQTYVTQQLIRAAEIRASANLAATKMSECVLGQSKRVDFC | |
| | | GKGYHLMSFPQAAPHGVVFLHVTYVPSQERNFTTAPAICHE | |
| | | GKAYFPREGVFVFNGTSWFITQRNFFSPQIITTDNTFVSGN | |
| | | CDVVIGIINNTVYDPLQPELDSFKEELDKYFKNHTSPDVDL | |
| | | GDISGINASVVNIQKEIDRLNEVAKNLNESLIDLQELGKYE | |
| | | QYIKWPWYVWLGFIAGLIAIVMVTILLCCMTSCCSCLKGAC | |
| | | SCGSCCKFDEDDSEPVLKGVKLHYT (SEQ ID NO: 7) | |
| SARS-CoV-2 | P0DTC2-1 | MFVFLVLLPLVSSQCVNLTTRTQLPPAYTNSFTRGVYYPDK | |
| | | VFRSSVLHSTQDLFLPFFSNVTWFHAIHVSGTNGTKRFDNP | |
| | | VLPFNDGVYFASTEKSNIIRGWIFGTTLDSKTQSLLIVNNA | |
| | | TNVVIKVCEFQFCNDPFLGVYYHKNNKSWMESEFRVYSSAN | |
| | | NCTFEYVSQPFLMDLEGKQGNFKNLREFVFKNIDGYFKIYS | |
| | | KHTPINLVRDLPQGFSALEPLVDLPIGINITRFQTLLALHR | |
| | | SYLTPGDSSSGWTAGAAAYYVGYLQPRTFLLKYNENGTITD | |
| | | AVDCALDPLSETKCTLKSFTVEKGIYQTSNFRVQPTESIVR | |
| | | FPNITNLCPFGEVENATRFASVYAWNRKRISNCVADYSVLY | |
| | | NSASFSTFKCYGVSPTKLNDLCFTNVYADSFVIRGDEVRQI | |
| | | APGQTGKIADYNYKLPDDFTGCVIAWNSNNLDSKVGGNYNY | |
| | | LYRLFRKSNLKPFERDISTEIYQAGSTPCNGVEGENCYFPL | |
| | | QSYGFQPTNGVGYQPYRVVVLSFELLHAPATVCGPKKSTNL | |
| | | VKNKCVNFNFNGLTGTGVLTESNKKFLPFQQFGRDIADTTD | |
| | | AVRDPQTLEILDITPCSFGGVSVITPGTNTSNQVAVLYQDV | |
| | | NCTEVPVAIHADQLTPTWRVYSTGSNVFQTRAGCLIGAEHV | |
| | | NNSYECDIPIGAGICASYQTQTNSPRRARSVASQSIIAYTM | |
| | | SLGAENSVAYSNNSIAIPTNFTISVTTEILPVSMTKTSVDC | |
| | | TMYICGDSTECSNLLLQYGSFCTQLNRALTGIAVEQDKNTQ | |
| | | EVFAQVKQIYKTPPIKDFGGFNFSQILPDPSKPSKRSFIED | |
| | | LLFNKVTLADAGFIKQYGDCLGDIAARDLICAQKFNGLTVL | |
| | | PPLLTDEMIAQYTSALLAGTITSGWTFGAGAALQIPFAMQM | |
| | | AYRFNGIGVTQNVLYENQKLIANQFNSAIGKIQDSLSSTAS | |
| | | ALGKLQDVVNQNAQALNTLVKQLSSNFGAISSVLNDILSRL | |
| | | DKVEAEVQIDRLITGRLQSLQTYVTQQLIRAAEIRASANLA | |
| | | ATKMSECVLGQSKRVDFCGKGYHLMSFPQSAPHGVVFLHVT | |
| | | YVPAQEKNFTTAPAICHDGKAHFPREGVFVSNGTHWFVTQR | |
| | | NFYEPQIITTDNTFVSGNCDVVIGIVNNTVYDPLQPELDSF | |
| | | KEELDKYFKNHTSPDVDLGDISGINASVVNIQKEIDRLNEV | |
| | | AKNLNESLIDLQELGKYEQYIKWPWYIWLGFIAGLIAIVMV | |
| | | TIMLCCMTSCCSCLKGCCSCGSCCKFDEDDSEPVLKGVKLH | |
| | | YT (SEQ ID NO: 8) | |
| SARS-CoV-2 | A0A7T8KZF1-1 | MFVFLVLLPLVSSQCVNLTTRTQLPPAYTNSFTRGVYYPDK | |
| Alpha | | VFRSSVLHSTQDLFLPFFSNVTWFHAISGTNGTKRFDNPVL | |
| (B.1.1.7) | | PFNDGVYFASTEKSNIIRGWIFGTTLDSKTQSLLIVNNATN | |
| | | VVIKVCEFQFCNDPFLGVYHKNNKSWMESEFRVYSSANNCT | |
| | | FEYVSQPFLMDLEGKQGNFKNLREFVFKNIDGYFKIYSKHT | |
| | | PINLVRDLPQGFSALEPLVDLPIGINITRFQTLLALHRSYL | |
| | | TPGDSSSGWTAGAAAYYVGYLQPRTFLLKYNENGTITDAVD | |
| | | CALDPLSETKCTLKSFTVEKGIYQTSNFRVQPTESIVRFPN | |
| | | ITNLCPFGEVFNATRFASVYAWNRKRISNCVADYSVLYNSA | |
| | | SFSTFKCYGVSPTKLNDLCFTNVYADSFVIRGDEVRQIAPG | |
| | | QTGKIADYNYKLPDDFTGCVIAWNSNNLDSKVGGNYNYLYR | |
| | | LFRKSNLKPFERDISTEIYQAGSTPCNGVEGFNCYFPLQSY | |
| | | GFQPTYGVGYQPYRVVVLSFELLHAPATVCGPKKSTNLVKN | |
| | | KCVNFNFNGLTGTGVLTESNKKFLPFQQFGRDIDDTTDAVR | |
| | | DPQTLEILDITPCSFGGVSVITPGTNTSNQVAVLYQGVNCT | |
| | | EVPVAIHADQLTPTWRVYSTGSNVFQTRAGCLIGAEHVNNS | |
| | | YECDIPIGAGICASYQTQTNSHRRARSVASQSIIAYTMSLG | |
| | | AENSVAYSNNSIAIPINFTISVTTEILPVSMTKTSVDCTMY | |
| | | ICGDSTECSNLLLQYGSFCTQLNRALTGIAVEQDKNTQEVF | |
| | | AQVKQIYKTPPIKDFGGFNFSQILPDPSKPSKRSFIEDLLF | |
| | | NKVTLADAGFIKQYGDCLGDIAARDLICAQKFNGLTVLPPL | |
| | | LTDEMIAQYTSALLAGTITSGWTFGAGAALQIPFAMQMAYR | |
| | | FNGIGVTQNVLYENQKLIANQFNSAIGKIQDSLSSTASALG | |
| | | KLQDVVNQNAQALNTLVKQLSSNFGAISSVLNDILARLDKV | |
| | | EAEVQIDRLITGRLQSLQTYVTQQLIRAAEIRASANLAATK | |
| | | MSECVLGQSKRVDFCGKGYHLMSFPQSAPHGVVFLHVTYVP | |
| | | AQEKNFTTAPAICHDGKAHFPREGVFVSNGTHWFVTQRNFY | |
| | | EPQIITTHNTFVSGNCDVVIGIVNNTVYDPLQPELDSFKEE | |
| | | LDKYFKNHTSPDVDLGDISGINASVVNIQKEIDRLNEVAKN | |
| | | LNESLIDLQELGKYEQYIKWPWYIWLGFIAGLIAIVMVTIM | |
| | | LCCMTSCCSCLKGCCSCGSCCKFDEDDSEPVLKGVKLHYT | |
| | | (SEQ ID NO: 9) | |
| SARS-CoV-2 | QRN78347.1 | MFVFLVLLPLVSSQCVNLTTRTQLPPAYTNSFTRGVYYPDK | |
| Beta (B.1.351) | | VFRSSVLHSTQDLFLPFFSNVTWFHAIHVSGTNGTKRFANP | |
| | | VLPFNDGVYFASTEKSNIIRGWIFGTTLDSKTQSLLIVNNA | |
| | | TNVVIKVCEFQFCNDPFLGVYYHKNNKSWMESEFRVYSSAN | |
| | | NCTFEYVSQPFLMDLEGKQGNFKNLREFVFKNIDGYFKIYS | |
| | | KHTPINLVRGLPQGFSALEPLVDLPIGINITRFQTLHRSYL | |
| | | TPGDSSSGWTAGAAAYYVGYLQPRTFLLKYNENGTITDAVD | |
| | | CALDPLSETKCTLKSFTVEKGIYQTSNFRVQPTESIVRFPN | |
| | | ITNLCPFGEVENATRFASVYAWNRKRISNCVADYSVLYNSA | |
| | | SFSTFKCYGVSPTKLNDLCFTNVYADSFVIRGDEVRQIAPG | |
| | | QTGNIADYNYKLPDDFTGCVIAWNSNNLDSKVGGNYNYLYR | |
| | | LFRKSNLKPFERDISTEIYQAGSTPCNGVKGFNCYFPLQSY | |
| | | GFQPTYGVGYQPYRVVVLSFELLHAPATVCGPKKSTNLVKN | |
| | | KCVNFNFNGLTGTGVLTESNKKFLPFQQFGRDIADTTDAVR | |
| | | DPQTLEILDITPCSFGGVSVITPGTNTSNQVAVLYQGVNCT | |
| | | EVPVAIHADQLTPTWRVYSTGSNVFQTRAGCLIGAEHVNNS | |
| | | YECDIPIGAGICASYQTQTNSPRRARSVASQSIIAYTMSLG | |
| | | VENSVAYSNNSIAIPTNFTISVTTEILPVSMTKTSVDCTMY | |
| | | ICGDSTECSNLLLQYGSFCTQLNRALTGIAVEQDKNTQEVF | |
| | | AQVKQIYKTPPIKDFGGFNFSQILPDPSKPSKRSFIEDLLF | |
| | | NKVTLADAGFIKQYGDCLGDIAARDLICAQKFNGLTVLPPL | |
| | | LTDEMIAQYTSALLAGTITSGWTFGAGAALQIPFAMQMAYR | |
| | | FNGIGVTQNVLYENQKLIANQFNSAIGKIQDSLSSTASALG | |
| | | KLQDVVNQNAQALNTLVKQLSSNFGAISSVLNDILSRLDKV | |
| | | EAEVQIDRLITGRLQSLQTYVTQQLIRAAEIRASANLAATK | |
| | | MSECVLGQSKRVDFCGKGYHLMSFPQSAPHGVVFLHVTYVP | |
| | | AQEKNFTTAPAICHDGKAHFPREGVFVSNGTHWFVTQRNFY | |
| | | EPQIITTDNTFVSGNCDVVIGIVNNTVYDPLQPELDSFKEE | |
| | | LDKYFKNHTSPDVDLGDISGINASVVNIQKEIDRLNEVAKN | |
| | | LNESLIDLQELGKYEQYIKWPWYIWLGFIAGLIAIVMVTIM | |
| | | LCCMTSCCSCLKGCCSCGSCCKFDEDDSEPVLKGVKLHYT | |
| | | (SEQ ID NO: 10) | |
| SARS-CoV-2 | A0A7U0LDJ8-1 | MFVFLVLLPLVSSQCVNFTNRTQLPSAYTNSFTRGVYYPDK | |
| Gamma (P.1) | | VFRSSVLHSTQDLFLPFFSNVTWFHAIHVSGTNGTKRFDNP | |
| | | VLPFNDGVYFASTEKSNIIRGWIFGTTLDSKTQSLLIVNNA | |
| | | TNVVIKVCEFQFCNYPFLGVYYHKNNKSWMESEFRVYSSAN | |
| | | NCTFEYVSQPFLMDLEGKQGNFKNLSEFVFKNIDGYFKIYS | |
| | | KHTPINLVRDLPQGFSALEPLVDLPIGINITRFQTLLALHR | |
| | | SYLTPGDSSSGWTAGAAAYYVGYLQPRTFLLKYNENGTITD | |
| | | AVDCALDPLSETKCTLKSFTVEKGIYQTSNFRVQPTESIVR | |
| | | FPNITNLCPFGEVFNATRFASVYAWNRKRISNCVADYSVLY | |
| | | NSASFSTFKCYGVSPTKLNDLCFTNVYADSFVIRGDEVRQI | |
| | | APGQTGTIADYNYKLPDDFTGCVIAWNSNNLDSKVGGNYNY | |
| | | LYRLFRKSNLKPFERDISTEIYQAGSTPCNGVKGFNCYFPL | |
| | | QSYGFQPTYGVGYQPYRVVVLSFELLHAPATVCGPKKSTNL | |
| | | VKNKCVNFNFNGLTGTGVLTESNKKFLPFQQFGRDIADTTD | |
| | | AVRDPQTLEILDITPCSFGGVSVITPGTNTSNQVAVLYQGV | |
| | | NCTEVPVAIHADQLTPTWRVYSTGSNVFQTRAGCLIGAEYV | |
| | | NNSYECDIPIGAGICASYQTQTNSPRRARSVASQSIIAYTM | |
| | | SLGAENSVAYSNNSIAIPTNFTISVTTEILPVSMTKTSVDC | |
| | | TMYICGDSTECSNLLLQYGSFCTQLNRALTGIAVEQDKNTQ | |
| | | EVFAQVKQIYKTPPIKDFGGFNFSQILPDPSKPSKRSFIED | |
| | | LLFNKVTLADAGFIKQYGDCLGDIAARDLICAQKFNGLTVL | |
| | | PPLLTDEMIAQYTSALLAGTITSGWTFGAGAALQIPFAMQM | |
| | | AYRFNGIGVTQNVLYENQKLIANQFNSAIGKIQDSLSSTAS | |
| | | ALGKLQDVVNQNAQALNTLVKQLSSNFGAISSVLNDILSRL | |
| | | DKVEAEVQIDRLITGRLQSLQTYVTQQLIRAAEIRASANLA | |
| | | AIKMSECVLGQSKRVDFCGKGYHLMSFPQSAPHGVVFLHVT | |
| | | YVPAQEKNFTTAPAICHDGKAHFPREGVFVSNGTHWFVTQR | |
| | | NFYEPQIITTDNTFVSGNCDVVIGIVNNTVYDPLQPELDSF | |
| | | KEELDKYFKNHTSPDVDLGDISGINASFVNIQKEIDRLNEV | |
| | | AKNLNESLIDLQELGKYEQYIKWPWYIWLGFIAGLIAIVMV | |
| | | TIMLCCMTSCCSCLKGCCSCGSCCKFDEDDSEPVLKGVKLH | |
| | | YT (SEQ ID NO: 11) | |
| SARS-CoV-2 | QQM19141.1 | MFVFLVLLPLVSIQCVNLTTRTQLPPAYTNSFTRGVYYPDK | |
| Epsilon | | VFRSSVLHSTQDLFLPFFSNVTWFHAIHVSGTNGTKRFDNP | |
| (B.1.427) | | VLPFNDGVYFASTEKSNIIRGWIFGTTLDSKTQSLLIVNNA | |
| | | TNVVIKVCEFQFCNDPFLGVYYHKNNKSCMESEFRVYSSAN | |
| | | NCTFEYVSQPFLMDLEGKQGNFKNLREFVFKNIDGYFKIYS | |
| | | KHTPINLVRDLPQGFSALEPLVDLPIGINITRFQTLLALHR | |
| | | SYLTPGDSSSGWTAGAAAYYVGYLQPRTFLLKYNENGTITD | |
| | | AVDCALDPLSETKCTLKSFTVEKGIYQTSNFRVQPTESIVR | |
| | | FPNITNLCPFGEVFNATRFASVYAWNRKRISNCVADYSVLY | |
| | | NSASFSTFKCYGVSPTKLNDLCFTNVYADSFVIRGDEVRQI | |
| | | APGQTGKIADYNYKLPDDFTGCVIAWNSNNLDSKVGGNYNY | |
| | | RYRLFRKSNLKPFERDISTEIYQAGSTPCNGVEGENCYFPL | |
| | | QSYGFQPTNGVGYQPYRVVVLSFELLHAPATVCGPKKSTNL | |
| | | VKNKCVNFNFNGLTGTGVLTESNKKFLPFQQFGRDIADTTD | |
| | | AVRDPQTLEILDITPCSFGGVSVITPGTNTSNQVAVLYQGV | |
| | | NCTEVPVAIHADQLTPTWRVYSTGSNVFQTRAGCLIGAEHV | |
| | | NNSYECDIPIGAGICASYQTQTNSPRRARSVASQSIIAYTM | |
| | | SLGAENSVAYSNNSIAIPTNFTISVTTEILPVSMTKTSVDC | |
| | | TMYICGDSTECSNLLLQYGSFCTQLNRALTGIAVEQDKNTQ | |
| | | EVFAQVKQIYKTPPIKDFGGFNFSQILPDPSKPSKRSFIED | |
| | | LLFNKVTLADAGFIKQYGDCLGDIAARDLICAQKFNGLTVL | |
| | | PPLLTDEMIAQYTSALLAGTITSGWTFGAGAALQIPFAMQM | |
| | | AYRFNGIGVTQNVLYENQKLIANQFNSAIGKIQDSLSSTAS | |
| | | ALGKLQDVVNQNAQALNTLVKQLSSNFGAISSVLNDILSRL | |
| | | DKVEAEVQIDRLITGRLQSLQTYVTQQLIRAAEIRASANLA | |
| | | ATKMSECVLGQSKRVDFCGKGYHLMSFPQSAPHGVVFLHVT | |
| | | YVPAQEKNFTTAPAICHDGKAHFPREGVFVSNGTHWFVTQR | |
| | | NFYEPQIITTDNTFVSGNCDVVIGIVNNTVYDPLQPELDSF | |
| | | KEELDKYFKNHTSPDVDLGDISGINASVVNIQKEIDRLNEV | |
| | | AKNLNESLIDLQELGKYEQYIKWPWYIWLGFIAGLIAIVMV | |
| | | TIMLCCMTSCCSCLKGCCSCGSCCKFDEDDSEPVLKGVKLH | |
| | | YT (SEQ ID NO: 12) | |
| SARS-CoV-2 | QWK65230.1 | MFVFLVLLPLVSSQCVNLRTRTQLPPAYTNSFTRGVYYPDK | |
| Delta | | VFRSSVLHSTQDLFLPFFSNVTWFHAIHVSGTNGTKRFDNP | |
| (B.1.617.2) | | VLPFNDGVYFASTEKSNIIRGWIFGTTLDSKTQSLLIVNNA | |
| | | NVVIKVCEFQFCNDPFLDVYYHKNNKSWMESGVYSSANNTC | |
| | | TFEYVSQPFLMDLEGKQGNFKNLREFVFKNIDGYFKIYSKH | |
| | | TPINLVRDLPQGFSALEPLVDLPIGINITRFQTLLALHRSY | |
| | | LTPGDSSSGWTAGAAAYYVGYLQPRTFLLKYNENGTITDAV | |
| | | DCALDPLSETKCTLKSFTVEKGIYQTSNFRVQPTESIVRFP | |
| | | NITNLCPFGEVFNATRFASVYAWNRKRISNCVADYSVLYNS | |
| | | ASFSTFKCYGVSPTKLNDLCFTNVYADSFVIRGDEVRQIAP | |
| | | GQTGKIADYNYKLPDDFTGCVIAWNSNNLDSKVGGNYNYRY | |
| | | RLFRKSNLKPFERDISTEIYQAGSKPCNGVEGENCYFPLQS | |
| | | YGFQPTNGVGYQPYRVVVLSFELLHAPATVCGPKKSTNLVK | |
| | | NKCVNFNFNGLTGTGVLTESNKKFLPFQQFGRDIADTTDAV | |
| | | RDPQTLEILDITPCSFGGVSVITPGTNTSNQVAVLYQGVNC | |
| | | TEVPVAIHADQLTPTWRVYSTGSNVFQTRAGCLIGAEHVNN | |
| | | SYECDIPIGAGICASYQTQTNSRRRARSVASQSIIAYTMSL | |
| | | GAENSVAYSNNSIAIPTNFTISVTTEILPVSMTKTSVDCTM | |
| | | YICGDSTECSNLLLQYGSFCTQLNRALTGIAVEQDKNTQEV | |
| | | FAQVKQIYKTPPIKDFGGFNFSQILPDPSKPSKRSFIEDLL | |
| | | FNKVTLADAGFIKQYGDCLGDIAARDLICAQKFNGLTVLPP | |
| | | LLTDEMIAQYTSALLAGTITSGWTFGAGAALQIPFAMQMAY | |
| | | RFNGIGVTQNVLYENQKLIANQFNSAIGKIQDSLSSTASAL | |
| | | GKLQNVVNQNAQALNTLVKQLSSNFGAISSVLNDILSRLDK | |
| | | VEAEVQIDRLITGRLQSLQTYVTQQLIRAAEIRASANLAAT | |
| | | KMSECVLGQSKRVDFCGKGYHLMSFPQSAPHGVVFLHVTYV | |
| | | PAQEKNFTTAPAICHDGKAHFPREGVFVSNGTHWFVTQRNF | |
| | | YEPQIITTDNTFVSGNCDVVIGIVNNTVYDPLQPELDSFKE | |
| | | ELDKYFKNHTSPDVDLGDISGINASVVNIQKEIDRLNEVAK | |
| | | NLNESLIDLQELGKYEQYIKWPWYIWLGFIAGLIAIVMVTI | |
| | | MLCCMTSCCSCLKGCCSCGSCCKFDEDDSEPVLKGVKLHYT | |
| | | (SEQ ID NO: 13) | |
| SARS-CoV-2 | EPI_ISL_ | AA Substitutions as compared to Severe | |
| Omicron | 6795834.2 | acute respiratory syndrome coronavirus | |
| (BA2), | | 2 isolate WIV04: Spike A27S, Spike | |
| Virus name: | | D405N, Spike D614G, Spike D796Y, Spike | |
| hCoV-19/ | | E484A, Spike G142D, Spike G339D, Spike | |
| South Africa/ | | H655Y, Spike K417N, Spike L24del, Spike | |
| CERI-KRISP- | | N440K, Spike N501Y, Spike N679K, Spike | |
| K032307/2021 | | N764K, Spike N969K, Spike P25del, Spike | |
| Type: beta- | | P26del, Spike P681H, Spike Q493R, Spike | |
| coronavirus | | Q498R, Spike Q954H, Spike R408S, Spike | |
| Clade: GR | | S371F, Spike S373P, Spike S375F, Spike | |
| Pango | | S477N, Spike T19I, Spike T376A, Spike | |
| Lineage: BA.2 | | T478K, Spike V213G, Spike Y505H, ET9I, | |
| (Pango | | M A63T, M D3G, M Q19E, N G204R, N R203K, | |
| v.3.1.17 | | N S413R, NS3 T223I, NSP1 S135R, NSP3 | |
| 2021-11-25), | | G489S, NSP3 T24I, NSP4 L264F, NSP4 T327I, | |
| BA.2-like | | NSP4 T492I, NSP5 P132H, NSP6 F108del, | |
| (Scorpio) | | NSP6 G107del, NSP6 S106del, NSP12 P323L, | |
| | | NSP13 R392C, NSP14 142V, NSP15 T112I | |
| SARS-CoV-2 | UFO69279.1 | MFVFLVLLPLVSSQCVNLTTRTQLPPAYTNSFTRGVYYPDK | |
| Omicron | | VFRSSVLHSTQDLFLPFFSNVTWFHVISGTNGTKRFDNPVL | |
| (BA1, | | PFNDGVYFASIEKSNIIRGWIFGTTLDSKTQSLLIVNNATN | |
| previously | | VIKVCEFQFCNDPFLDHKNNKSWMESEFRVYSSANNCTFEV | |
| B.1.1.529) | | YVSQPFLMDLEGKQGNFKNLREFVFKNIDGYFKIYSKHTPI | |
| | | IVREPEDLPQGFSALEPLVDLPIGINITRFQTLLALHRSYL | |
| | | TPGDSSSGWTAGAAAYYVGYLQPRTFLLKYNENGTITDAVD | |
| | | CALDPLSETKCTLKSFTVEKGIYQTSNFRVQPTESIVRFPN | |
| | | ITNLCPFDEVFNATRFASVYAWNRKRISNCVADYSVLYNLA | |
| | | PFFTFKCYGVSPTKLNDLCFTNVYADSFVIRGDEVRQIAPG | |
| | | QTGNIADYNYKLPDDFTGCVIAWNSNKLDSKVSGNYNYLYR | |
| | | LFRKSNLKPFERDISTEIYQAGNKPCNGVAGFNCYFPLRSY | |
| | | SFRPTYGVGHQPYRVVVLSFELLHAPATVCGPKKSTNLVKN | |
| | | KCVNFNFNGLKGTGVLTESNKKFLPFQQFGRDIADTTDAVR | |
| | | DPQTLEILDITPCSFGGVSVITPGTNTSNQVAVLYQGVNCT | |
| | | EVPVAIHADQLTPTWRVYSTGSNVFQTRAGCLIGAEYVNNS | |
| | | YECDIPIGAGICASYQTQTKSHRRARSVASQSIIAYTMSLG | |
| | | AENSVAYSNNSIAIPTNFTISVTTEILPVSMTKTSVDCTMY | |
| | | ICGDSTECSNLLLQYGSFCTQLKRALTGIAVEQDKNTQEVF | |
| | | AQVKQIYKTPPIKYFGGFNFSQILPDPSKPSKRSFIEDLLF | |
| | | NKVTLADAGFIKQYGDCLGDIAARDLICAQKFKGLTVLPPL | |
| | | LTDEMIAQYTSALLAGTITSGWTFGAGAALQIPFAMQMAYR | |
| | | FNGIGVTQNVLYENQKLIANQFNSAIGKIQDSLSSTASALG | |
| | | KLQDVVNHNAQALNTLVKQLSSKFGAISSVLNDIFSRLDKV | |
| | | EAEVQIDRLITGRLQSLQTYVTQQLIRAAEIRASANLAATK | |
| | | MSECVLGQSKRVDFCGKGYHLMSFPQSAPHGVVFLHVTYVP | |
| | | AQEKNFTTAPAICHDGKAHFPREGVFVSNGTHWFVTQRNFY | |
| | | EPQIITTDNTFVSGNCDVVIGIVNNTVYDPLQPELDSFKEE | |
| | | LDKYFKNHTSPDVDLGDISGINASVVNIQKEIDRLNEVAKN | |
| | | LNESLIDLQELGKYEQYIKWPWYIWLGFIAGLIAIVMVTIM | |
| | | LCCMTSCCSCLKGCCSCGSCCKFDEDDSEPVLKGVKLHYT | |
| | | (SEQ ID NO:1) (SEQ ID NO: 1) | |
| SARS-CoV-2 | UJQ54091.1 | MFVFLVLLPL VSSQCVNLIT RTQSYTNSFT | 60 |
| | | RGVYYPDKVF RSSVLHSTQD LFLPFFSNVT | |
| | | WFHAIHVSGT NGTKRFDNPV LPFNDGVYFA | 120 |
| | | STEKSNIIRG WIFGTTLDSK TQSLLIVNNA | |
| | | TNVVIKVCEF QFCNDPFLDV YYHKNNKSWM | 180 |
| | | ESEFRVYSSA NNCTFEYVSQ PFLMDLEGKQ | |
| | | GNFKNLREFV FKNIDGYFKI YSKHTPINLG | 240 |
| | | RDLPQGFSAL EPLVDLPIGI NITRFQTLLA | |
| | | LHRSYLTPGD SSSGWTAGAA AYYVGYLQPR | 300 |
| | | TFLLKYNENG TITDAVDCAL DPLSETKCTL | |
| | | KSFTVEKGIY QTSNFRVQPT ESIVRFPNIT | 360 |
| | | NLCPFDEVEN ATRFASVYAW NRKRISNCVA | |
| | | DYSVLYNFAP FFAFKCYGVS PTKLNDLCFT | 420 |
| | | NVYADSFVIR GNEVSQIAPG QTGNIADYNY | |
| | | KLPDDFTGCV IAWNSNKLDS KVGGNYNYLY | 480 |
| | | RLFRKSNLKP FERDISTEIY QAGNKPCNGV | |
| | | AGFNCYFPLR SYGFRPTYGV GHQPYRVVVL | 540 |
| | | SFELLHAPAT VCGPKKSTNL VKNKCVNFNF | |
| | | NGLTGTGVLT ESNKKFLPFQ QFGRDIADTT | 600 |
| | | DAVRDPQTLE ILDITPCSFG GVSVITPGTN | |
| | | TSNQVAVLYQ GVNCTEVPVA IHADQLTPTW | 660 |
| | | RVYSTGSNVF QTRAGCLIGA EYVNNSYECD | |
| | | IPIGAGICAS YQTQTKSHRR ARSVASQSII | 720 |
| | | AYTMSLGAEN SVAYSNNSIA IPTNFTISVT | |
| | | TEILPVSMTK TSVDCTMYIC GDSTECSNLL | 780 |
| | | LQYGSFCTQL KRALTGIAVE QDKNTQEVFA | |
| | | QVKQIYKTPP IKYFGGFNFS QILPDPSKPS | 840 |
| | | KRSFIEDLLF NKVTLADAGF IKQYGDCLGD | |
| | | IAARDLICAQ KFNGLTVLPP LLTDEMIAQY | 900 |
| | | TSALLAGTIT SGWTFGAGAA LQIPFAMQMA | |
| | | YRENGIGVTQ NVLYENQKLI ANQFNSAIGK | 960 |
| | | IQDSLSSTAS ALGKLQDVVN HNAQALNTLV | |
| | | KQLSSKFGAI SSVLNDILSR LDKVEAEVQI | 1020 |
| | | DRLITGRLQS LQTYVTQQLI RAAEIRASAN | |
| | | LAATKMSECV LGQSKRVDFC GKGYHLMSFP | 1080 |
| | | QSAPHGVVFL HVTYVPAQEK NFTTAPAICH | |
| | | DGKAHFPREG VFVSNGTHWF VTQRNFYEPQ | 1140 |
| | | IITTDNTFVS GNCDVVIGIV NNTVYDPLQP | |
| | | ELDSFKEELD KYFKNHTSPD VDLGDISGIN | 1200 |
| | | ASVVNIQKEI DRLNEVAKNL NESLIDLQEL | |
| | | GKYEQYIKWP WYIWLGFIAG LIAIVMVTIM | 1260 |
| | | LCCMTSCCSC LKGCCSCGSC CKFDEDDSEP | |
| | | VLKGVKLHYT (SEQ ID NO: 14) | 1270 |

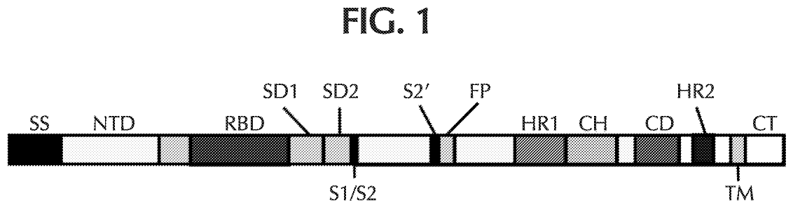

A schematic of a coronavirus spike protein having a S1 subunit and a S2 subunit is shown in FIG. 1. The S1 subunit comprises a leader, or signal, sequence (SS), a N-terminal domain (NTD) and a receptor binding domain (RBD). The S2 subunit comprises heptad repeat regions (HR1 and HR2) and a transmembrane domain (TM). Modifications to the spike protein sequence may be made anywhere within the sequence as described herein, but in some aspects, modifications are made in the NTD or RBD or a sequence linking the NTD and RBD. In some aspects, modifications are made in an amino acid sequence linking the first heptad repeat region to the second heptad repeat region. In some aspects, modifications are made in an amino acid sequence at the interface of the S1 and S2 subunits. For example, in some aspects, amino acid substitutions are made in the NTD and/or RBD and/or a sequence linking the NTD and RBD and/or amino acid substitutions are made in an amino acid sequence linking the first heptad repeat region to the second heptad repeat region and/or amino acid substitutions are made in an amino acid sequence at the interface of the S1 and S2 subunits.

### B. Specific Modifications & Combinations Thereof

The following Table 2 lists the various combinations of modifications that can be made to amino acids in the coronavirus spike protein sequences disclosed above to produce isolated immunogenic polypeptides comprising variant coronavirus spike proteins that are variants of native coronavirus spike proteins or fragments thereof. A “+” symbol indicates the inclusion of the specified modification in a specific variant coronavirus spike protein. In some instances, the variant contains any combination of the modifications in the following Table 2. The amino acid position corresponds with the amino acid position in SEQ ID. NO: 1, and can be used to determine the corresponding amino acid in other coronavirus spike proteins.

TABLE 2
Non-Inclusive Coronavirus Spike Protein Modification Combinations

| | |
|---|---|
| Amino Acid | Modification Status of Variant (Variant #) |

| Position | 1 | 2 | 3 | 4 | 5 | 6 | 7 | 8 | 9 | 10 | 11 | 12 | 13 | 14 | 15 | 16 | 17 | 18 |
|---|---|---|---|---|---|---|---|---|---|---|---|---|---|---|---|---|---|---|
| 326 | + | + | + | + | + | + | + | + | + | + | + | + | + | + | + | + | + | + |
| 364 | | + | + | + | + | + | + | + | + | + | + | + | + | + | + | + | + | + |
| 567 | | | + | + | + | + | + | + | + | + | + | + | + | + | + | + | + | + |
| 611 | | | | + | + | + | + | + | + | + | + | + | + | + | + | + | + | + |
| 814 | | | | | + | + | + | + | + | + | + | + | + | + | + | + | + | + |
| 840 | | | | | | + | + | + | + | + | + | + | + | + | + | + | + | + |
| 851 | | | | | | | + | + | + | + | + | + | + | + | + | + | + | + |
| 889 | | | | | | | | + | + | + | + | + | + | + | + | + | + | + |
| 896 | | | | | | | | | + | + | + | + | + | + | + | + | + | + |
| 939 | | | | | | | | | | + | + | + | + | + | + | + | + | + |
| 957 | | | | | | | | | | | + | + | + | + | + | + | + | + |
| 977 | | | | | | | | | | | | + | + | + | + | + | + | + |
| 981 | | | | | | | | | | | | | + | + | + | + | + | + |
| 982 | | | | | | | | | | | | | | + | + | + | + | + |
| 983 | | | | | | | | | | | | | | | + | + | + | + |
| 984 | | | | | | | | | | | | | | | | + | + | + |
| 986 | | | | | | | | | | | | | | | | | + | + |
| 989 | | | | | | | | | | | | | | | | | | + |

| Amino Acid | Modification Status of Variant (Variant #) | | | | | | | | | | | | | | | | |
|---|---|---|---|---|---|---|---|---|---|---|---|---|---|---|---|---|---|
| Position | 19 | 20 | 21 | 22 | 23 | 24 | 25 | 26 | 27 | 28 | 29 | 30 | 31 | 32 | 33 | 34 | 35 |
| 364 | + | + | + | + | + | + | + | + | + | + | + | + | + | + | + | + | + |
| 567 | | + | + | + | + | + | + | + | + | + | + | + | + | + | + | + | + |
| 611 | | | + | + | + | + | + | + | + | + | + | + | + | + | + | + | + |
| 814 | | | | + | + | + | + | + | + | + | + | + | + | + | + | + | + |
| 840 | | | | | + | + | + | + | + | + | + | + | + | + | + | + | + |
| 851 | | | | | | + | + | + | + | + | + | + | + | + | + | + | + |
| 889 | | | | | | | + | + | + | + | + | + | + | + | + | + | + |
| 896 | | | | | | | | + | + | + | + | + | + | + | + | + | + |
| 939 | | | | | | | | | + | + | + | + | + | + | + | + | + |
| 957 | | | | | | | | | | + | + | + | + | + | + | + | + |
| 977 | | | | | | | | | | | + | + | + | + | + | + | + |
| 981 | | | | | | | | | | | | + | + | + | + | + | + |
| 982 | | | | | | | | | | | | | + | + | + | + | + |
| 983 | | | | | | | | | | | | | | + | + | + | + |
| 984 | | | | | | | | | | | | | | | + | + | + |
| 986 | | | | | | | | | | | | | | | | + | + |
| 989 | | | | | | | | | | | | | | | | | + |

| Amino Acid | Modification Status of Variant (Variant #) | | | | | | | | | | | | | | | |
|---|---|---|---|---|---|---|---|---|---|---|---|---|---|---|---|---|
| Position | 36 | 37 | 38 | 39 | 40 | 41 | 42 | 43 | 44 | 45 | 46 | 47 | 48 | 49 | 50 | 51 |
| 567 | + | + | + | + | + | + | + | + | + | + | + | + | + | + | + | + |
| 611 | | + | + | + | + | + | + | + | + | + | + | + | + | + | + | + |
| 814 | | | + | + | + | + | + | + | + | + | + | + | + | + | + | + |
| 840 | | | | + | + | + | + | + | + | + | + | + | + | + | + | + |
| 851 | | | | | + | + | + | + | + | + | + | + | + | + | + | + |
| 889 | | | | | | + | + | + | + | + | + | + | + | + | + | + |
| 896 | | | | | | | + | + | + | + | + | + | + | + | + | + |
| 939 | | | | | | | | + | + | + | + | + | + | + | + | + |
| 957 | | | | | | | | | + | + | + | + | + | + | + | + |
| 977 | | | | | | | | | | + | + | + | + | + | + | + |
| 981 | | | | | | | | | | | + | + | + | + | + | + |
| 982 | | | | | | | | | | | | + | + | + | + | + |
| 983 | | | | | | | | | | | | | + | + | + | + |
| 984 | | | | | | | | | | | | | | + | + | + |
| 986 | | | | | | | | | | | | | | | + | + |
| 989 | | | | | | | | | | | | | | | | + |

| | |
|---|---|
| Amino Acid | Modification Status of Variant (Variant #) |

| Position | 52 | 53 | 54 | 55 | 56 | 57 | 58 | 59 | 60 | 61 | 62 | 63 | 64 | 65 | 66 |
|---|---|---|---|---|---|---|---|---|---|---|---|---|---|---|---|
| 611 | + | + | + | + | + | + | + | + | + | + | + | + | + | + | + |
| 814 | | + | + | + | + | + | + | + | + | + | + | + | + | + | + |
| 840 | | | + | + | + | + | + | + | + | + | + | + | + | + | + |
| 851 | | | | + | + | + | + | + | + | + | + | + | + | + | + |
| 889 | | | | | + | + | + | + | + | + | + | + | + | + | + |
| 896 | | | | | | + | + | + | + | + | + | + | + | + | + |
| 939 | | | | | | | + | + | + | + | + | + | + | + | + |
| 957 | | | | | | | | + | + | + | + | + | + | + | + |
| 977 | | | | | | | | | + | + | + | + | + | + | + |
| 981 | | | | | | | | | | + | + | + | + | + | + |
| 982 | | | | | | | | | | | + | + | + | + | + |
| 983 | | | | | | | | | | | | + | + | + | + |
| 984 | | | | | | | | | | | | | + | + | + |
| 986 | | | | | | | | | | | | | | + | + |
| 989 | | | | | | | | | | | | | | | + |

| | |
|---|---|
| Amino Acid | Modification Status of Variant (Variant #) |

| Position | 67 | 68 | 69 | 70 | 71 | 72 | 73 | 74 | 75 | 76 | 77 | 78 | 79 | 80 |
|---|---|---|---|---|---|---|---|---|---|---|---|---|---|---|
| 814 | + | + | + | + | + | + | + | + | + | + | + | + | + | + |
| 840 | | + | + | + | + | + | + | + | + | + | + | + | + | + |
| 851 | | | + | + | + | + | + | + | + | + | + | + | + | + |
| 889 | | | | + | + | + | + | + | + | + | + | + | + | + |
| 896 | | | | | + | + | + | + | + | + | + | + | + | + |
| 939 | | | | | | + | + | + | + | + | + | + | + | + |
| 957 | | | | | | | + | + | + | + | + | + | + | + |
| 977 | | | | | | | | + | + | + | + | + | + | + |
| 981 | | | | | | | | | + | + | + | + | + | + |
| 982 | | | | | | | | | | + | + | + | + | + |
| 983 | | | | | | | | | | | + | + | + | + |
| 984 | | | | | | | | | | | | + | + | + |
| 986 | | | | | | | | | | | | | + | + |
| 989 | | | | | | | | | | | | | | + |

| | |
|---|---|
| Amino Acid | Modification Status of Variant (Variant #) |

| Position | 81 | 82 | 83 | 84 | 85 | 86 | 87 | 88 | 89 | 90 | 91 | 92 | 93 |
|---|---|---|---|---|---|---|---|---|---|---|---|---|---|
| 840 | + | + | + | + | + | + | + | + | + | + | + | + | + |
| 851 | | + | + | + | + | + | + | + | + | + | + | + | + |
| 889 | | | + | + | + | + | + | + | + | + | + | + | + |
| 896 | | | | + | + | + | + | + | + | + | + | + | + |
| 939 | | | | | + | + | + | + | + | + | + | + | + |
| 957 | | | | | | + | + | + | + | + | + | + | + |
| 977 | | | | | | | + | + | + | + | + | + | + |
| 981 | | | | | | | | + | + | + | + | + | + |
| 982 | | | | | | | | | + | + | + | + | + |
| 983 | | | | | | | | | | + | + | + | + |
| 984 | | | | | | | | | | | + | + | + |
| 986 | | | | | | | | | | | | + | + |
| 989 | | | | | | | | | | | | | + |

| Amino Acid | Modification Status of Variant (Variant #) | | | | | | | | | | | |
|---|---|---|---|---|---|---|---|---|---|---|---|---|
| Position | 94 | 95 | 96 | 97 | 98 | 99 | A1 | A2 | A3 | A4 | A5 | A6 |
| 851 | + | + | + | + | + | + | + | + | + | + | + | + |
| 889 | | + | + | + | + | + | + | + | + | + | + | + |
| 896 | | | + | + | + | + | + | + | + | + | + | + |
| 939 | | | | + | + | + | + | + | + | + | + | + |
| 957 | | | | | + | + | + | + | + | + | + | + |
| 977 | | | | | | + | + | + | + | + | + | + |
| 981 | | | | | | | + | + | + | + | + | + |
| 982 | | | | | | | | + | + | + | + | + |
| 983 | | | | | | | | | + | + | + | + |
| 984 | | | | | | | | | | + | + | + |
| 986 | | | | | | | | | | | + | + |
| 989 | | | | | | | | | | | | + |

| Amino Acid | Modification Status of Variant (Variant #) | | | | | | | | | | |
|---|---|---|---|---|---|---|---|---|---|---|---|
| Position | A7 | A8 | A9 | B1 | B2 | B3 | B4 | B5 | B6 | B7 | B8 |
| 889 | + | + | + | + | + | + | + | + | + | + | + |
| 896 | | + | + | + | + | + | + | + | + | + | + |
| 939 | | | + | + | + | + | + | + | + | + | + |
| 957 | | | | + | + | + | + | + | + | + | + |
| 977 | | | | | + | + | + | + | + | + | + |
| 981 | | | | | | + | + | + | + | + | + |
| 982 | | | | | | | + | + | + | + | + |
| 983 | | | | | | | | + | + | + | + |
| 984 | | | | | | | | | + | + | + |
| 986 | | | | | | | | | | + | + |
| 989 | | | | | | | | | | | + |

| | |
|---|---|
| Amino Acid | Modification Status of Variant (Variant #) |

| Position | B9 | C1 | C2 | C3 | C4 | C5 | C6 | C7 | C8 | C9 |
|---|---|---|---|---|---|---|---|---|---|---|
| 896 | + | + | + | + | + | + | + | + | + | + |
| 939 | | + | + | + | + | + | + | + | + | + |
| 957 | | | + | + | + | + | + | + | + | + |
| 977 | | | | + | + | + | + | + | + | + |
| 981 | | | | | + | + | + | + | + | + |
| 982 | | | | | | + | + | + | + | + |
| 983 | | | | | | | + | + | + | + |
| 984 | | | | | | | | + | + | + |
| 986 | | | | | | | | | + | + |
| 989 | | | | | | | | | | + |

| Amino Acid | Modification Status of Variant (Variant #) | | | | | | | | |
|---|---|---|---|---|---|---|---|---|---|
| Position | D1 | D2 | D3 | D4 | D5 | D6 | D7 | D8 | D9 |
| 939 | + | + | + | + | + | + | + | + | + |
| 957 | | + | + | + | + | + | + | + | + |
| 977 | | | + | + | + | + | + | + | + |
| 981 | | | | + | + | + | + | + | + |
| 982 | | | | | + | + | + | + | + |
| 983 | | | | | | + | + | + | + |
| 984 | | | | | | | + | + | + |
| 986 | | | | | | | | + | + |
| 989 | | | | | | | | | + |

| | |
|---|---|
| Amino Acid | Modification Status of Variant (Variant #) |

| Position | E1 | E2 | E3 | E4 | E5 | E6 | E7 | E8 |
|---|---|---|---|---|---|---|---|---|
| 957 | + | + | + | + | + | + | + | + |
| 977 | | + | + | + | + | + | + | + |
| 981 | | | + | + | + | + | + | + |
| 982 | | | | + | + | + | + | + |
| 983 | | | | | + | + | + | + |
| 984 | | | | | | + | + | + |
| 986 | | | | | | | + | + |
| 989 | | | | | | | | + |

| Amino Acid | Modification Status of Variant (Variant #) | | | | | | |
|---|---|---|---|---|---|---|---|
| Position | E9 | F1 | F2 | F3 | F4 | F5 | F6 |
| 977 | + | + | + | + | + | + | + |
| 981 | | + | + | + | + | + | + |
| 982 | | | + | + | + | + | + |
| 983 | | | | + | + | + | + |
| 984 | | | | | + | + | + |
| 986 | | | | | | + | + |
| 989 | | | | | | | + |

| | |
|---|---|
| Amino Acid | Modification Status of Variant (Variant #) |

| Position | F7 | F8 | F9 | G1 | G2 | G3 |
|---|---|---|---|---|---|---|
| 981 | + | + | + | + | + | + |
| 982 | | + | + | + | + | + |
| 983 | | | + | + | + | + |
| 984 | | | | + | + | + |
| 986 | | | | | + | + |
| 989 | | | | | | + |

| Amino Acid | Modification Status of Variant (Variant #) | | | | |
|---|---|---|---|---|---|
| Position | G4 | G5 | G6 | G7 | G8 |
| 982 | + | + | + | + | + |
| 983 | | + | + | + | + |
| 984 | | | + | + | + |
| 986 | | | | + | + |
| 989 | | | | | + |

| | |
|---|---|
| Amino Acid | Modification Status of Variant (Variant #) |

| Position | G9 | H1 | H2 | H3 |
|---|---|---|---|---|
| 983 | + | + | + | + |
| 984 | | + | + | + |
| 986 | | | + | + |
| 989 | | | | + |

| Amino Acid | Modification Status of Variant (Variant #) | | |
|---|---|---|---|
| Position | H4 | H5 | H6 |
| 984 | + | + | + |
| 986 | | + | + |
| 989 | | | + |

| | |
|---|---|
| Amino Acid | Modification Status of Variant (Variant #) |

| Position | H7 | H8 |
|---|---|---|
| 986 | + | + |
| 989 | | + |

| Amino Acid | Modification Status of Variant (Variant #) |
|---|---|
| Position | H9 |
| 989 | + |

The amino acid numbers in the above Table 2 correspond to the amino acid position in the sequence of the SARS-CoV-2 omicron BA1 variant spike protein (SEQ ID NO: 1) and identify the position of a modification that can be made in any coronavirus spike protein. As one example, the amino acid in SARS-CoV corresponding to the amino acid at position 326 of SEQ ID NO:1 may be modified to produce an isolated immunogenic polypeptide comprising a variant coronavirus spike protein. As another example, the amino acid in MERS-CoV corresponding to the amino acid at position 326 of SEQ ID NO:1 may be modified to produce an isolated immunogenic polypeptide comprising a variant coronavirus spike protein. The amino acids in each human coronavirus spike protein sequence and the corresponding position of that amino acid with respect to SEQ ID NO:1 can be determined based an alignment of the protein sequences. Below in Table 3 is an alignment of human coronavirus spike protein sequences (e.g., the spike protein sequences of NL63-CoV, 229E-CoV, OC43-CoV, HKU1-CoV, MERS-CoV, SARS-CoV, and SARS-CoV-2) with SEQ ID NO:1 (Omicron, corresponding to the SARS-CoV-2 BA1 omicron variant spike protein). The highlighted positions in the below alignment correspond to the location of the amino acids to be modified identified in the Table 2 above.

TABLE 3

| | | |
|---|---|---|
| NL63 | --MKLFLILLVLPLASCFF-----TCNSNA-N----LSMLQLGVPDNSSTIVTGLLPTHW | 48 |
| 229E | ------------------------------------------------------------ | 0 |
| OC43 | --MFLILLISLP-TAFAVIGDLKCTSDNIND--------KDTGPPPIST----------- | 38 |
| HKU1 | ---MLLIIFILP-TTLAVIGDFNCTNFAIND--------LNTTVPRISE----------- | 37 |
| MERS | MIHSVFLLMFLLTPTESYVDVGPDSVKSACIEVDIQQTFFDKTWP---R----------- | 46 |
| SARS-CoV | ----MFIFLLFLTL-T----------SGSDLDR--CTTFDDVQAP---N----------- | 29 |
| SARS-CoV-2 | ----MFVFLVLLPL-V----------SSQCVNL--TT--RTQLPP---A----------- | 27 |
| Omicron | ----MFVFLVLLPL-V----------SSQCVNL--TT--RTQLPP---A----------- | 27 |
| NL63 | FCANQSTSVYSANGFFYIDVGNHRSAFALHTGYYDA---NQYYIYVTNEIGLNASVTLKI | 105 |
| 229E | ------------------------------------------------------------ | 0 |
| OC43 | ----DTVDVTNGLGTYYVLDRVYLNTTLFLNGYYPTSGSTYRNM------ALKGSVLLSR | 88 |
| HKU1 | ----YVVDVSYGLGTYYILDRVYLNTTILFTGYFPKSGANFRDL------SLKGTTYLST | 87 |
| MERS | ----P-IDVSKADGIIYPQGRTYSNITITYQGLF-PYQGDHGDMYVYSAGHATGTTPQKL | 100 |
| SARS-CoV | ----YTQHTSSMRGVYYPDEIFRSDTLYLTQDLFLPFYSNVTG---FHTIN-------HT | 75 |
| SARS-CoV-2 | ----Y--TNSFTRGVYYPDKVFRSSVLHSTQDLFLPFFSNVTW---FHAIHVSGTNGTKR | 78 |
| Omicron | ----Y--TNSFTRGVYYPDKVFRSSVLHSTQDLFLPFFSNVTW---FHVI--SGTNGTKR | 76 |
| NL63 | --CKFSRNTTFDFLSNASSSFDCIVNLLFTEQLGAPLGITISGETVRLHLYNVTRTFYVP | 163 |
| 229E | ----------------------------------------------------------- | 0 |
| OC43 | --LWFKPPFLSDFINGIFAKVKNTK--V-------IKDR---------VMYSEFPAITIG | 128 |
| HKU1 | --LWYQKPFLSDFNNGIFSRVKNTK--L-------YVNK---------TLYSEFSTIVIG | 127 |
| MERS | FVANYSQD-VKQFANGFVVRIGAAA--N-------STGTVIISPSTSATIRKIYPAFMLG | 150 |
| SARS-CoV | ----FGNP-VIPFKDGIYFAAT----------------------EK----SNVVRGWVFG | 104 |
| SARS-CoV-2 | ----FDNP-VLPFNDGVYFAST----------------------EK----SNIIRGWIFG | 107 |
| Omicron | ----FDNP-VLPFNDGVYFASI----------------------EK----SNIIRGWIFG | 105 |
| NL63 | AAYKLTKL-SVKCYFNYSCVFSVVNATVTVNVTTHNGRVVNYTVCDDCNGYTDNIFSVQQ | 222 |
| 229E | -------------MFVLLVAYA---LLHIAGCQTTNGLNTSYSVCNGCVGYSENVFAVES | 44 |
| OC43 | STFVNTSY---------SVVV----QPRTINS-TQDG----DNKLQGLLEVSVCQY---- | 166 |
| HKU1 | SVFINNSY---------TIVV----QPH-----------------NGVLEITACQY---- | 153 |
| MERS | SSVGNFSDGKMGRFFNHTLVL----LPDG---------------CGTLLRAFYCILEPRS | 191 |
| SARS-CoV | STMNNKSQ---------SVII----INNS---------------TNVVIRA--CNF---- | 130 |
| SARS-CoV-2 | TTLDSKTQ---------SLLI----VNNA---------------TNVVIKV--CEF---- | 133 |
| Omicron | TTLDSKTQ---------SLLI----VNNA---------------TNVVIKV--CEF---- | 131 |
| NL63 | DGRIPNGFPFNNWFLLTNGSTLVDGVSRLYQPLRLTCLWPVPGLKSSTGFVYFNATGSDV | 282 |
| 229E | GGYIPSDFAFNNWFLLTNTSSVVDGVVRSFQPLLLNCLWSVSGLRFTTGFVYFNGTGR-G | 103 |
| OC43 | -------------NMCEYPQTICHPNL----GNHRKELWHLD--------T--G----VV | 195 |
| HKU1 | -------------TMCEYPHTICKS-K----GSSRNESWHFD--------K--S----EP | 181 |
| MERS | GNHCPAGNSYTSFATYHTPATDCSDGN----YNRNASLNSFK--------EYFN----LR | 235 |
| SARS-CoV | -------------ELCDNPFFAVSKPM----GT----QTHTM--------IFDN----AF | 157 |
| SARS-CoV-2 | -------------QFCNDPFLGVYYHK----NNKSWMESEFR--------VYSS----AN | 164 |
| Omicron | -------------QFCNDPFLD---HK----NNKSWMESEFR--------VYSS----AN | 159 |
| NL63 | NCN---GYQHNSVVDVMR-YNLNFSA---NSLDNLKSGVIVFKTLQYDVLFYCSNSSSG- | 334 |
| 229E | DCK---GFSSDVLSDVIR-YNLNFEE-------NLRRGTILFKTSYGVVVFYCTNNTLV- | 151 |
| OC43 | SCLYKRNFTYDVNAD-----------YLYFHFYQEGGTFYAYFTDTGVVT---------- | 234 |
| HKU1 | LCLFKKNFTYNVSTD-----------WLYFHFYQERGTFYAYYADSGMPT---------- | 220 |
| MERS | NCTFM--YTYNITEDEIL------------------EWFGITQTAQGVHLFSSRYVDLYG | 275 |
| SARS-CoV | NCTFE--YISDAFSLDVSEKSGNFKHLREFVFKNKDGFLYVYKGYQPIDV--V--RDLPS | 211 |
| SARS-CoV-2 | NCTFE--YVSQPFLMDLEGKQGNFKNLREFVFKNIDGYFKIYSKHTPINL--V--RDLPQ | 218 |
| Omicron | NCTFE--YVSQPFLMDLEGKQGNFKNLREFVFKNIDGYFKIYSKHTPIIVREP--EDLPQ | 215 |
| | * : : | |
| NL63 | -----VLDTTIPFGPSSQPYYCFINSTINTTHVSTFVGILPPTVREIVVARTGQFYINGF | 389 |
| 229E | -----SGDAHIPFGTVLGNFYCFVNTTIGNETTSAFVGALPKTVREFVISRTGHFYINGY | 206 |
| OC43 | -----KFLFNVYLGMALSHYYVM-----------------PLTCN--------------- | 257 |
| HKU1 | -----TFLFSLYLGTLLSHYYVL-----------------PLTCN--------------- | 243 |
| MERS | G--NMFQFATLPVYDTIKYYSII-----------------PHSIR---S----------- | 302 |
| SARS-CoV | GFNTLKPIFKLPLGINITNFRAI-----------------LTAFS--------------- | 239 |
| SARS-CoV-2 | GFSALEPLVDLPIGINITRFQTL-----------------LALHRSYLT----------- | 250 |
| Omicron | GFSALEPLVDLPIGINITRFQTL-----------------LALHRSYLT----------- | 247 |
| | : . : : | |
| NL63 | KYFDLGFIEAVNFNVTTASATDFWTVAFATFVDVLVNVSATNIQNLLYCD-SPFEKLQCE | 448 |
| 229E | RYFTLGNVEAVNFNVTTAETTDFCTVALASYADVLVNVSQTSIANIIYCN-SVINRLRCD | 265 |
| OC43 | ------S----------KLTLEYWVTPLTSRQYLLAFNQDGIIFNAEDCMSDFMSEIKCK | 301 |
| HKU1 | ------A----ISSNTDNETLQYWVTPLSKRQYLLKFDNRGVITNAVDCSSSFFSEIQCK | 293 |
| MERS | ----IQS----DRKAW----AAFYVYKLQPLTFLLDFSVDGYIRRAIDCGFNDLSQLHCS | 350 |
| SARS-CoV | ------P----AQDIWGTSAAAYFVGYLKPTTFMLKYDENGTITDAVDCSQNPLAELKCS | 289 |
| SARS-CoV-2 | ----PGD----SSSGWTAGAAAYYVGYLQPRTFLLKYNENGTITDAVDCALDPLSETKCT | 302 |
| Omicron | ----PGD----SSSGWTAGAAAYYVGYLQPRTFLLKYNENGTITDAVDCALDPLSETKCT | 299 |
| | : . : :* * * . : . :* | |
| NL63 | HLQFGLQDGFYSANFLDDNVLPETYVALPI--------------------YYQHTDINFT | 488 |
| 229E | QLSFDVPDGFYSTSPIQSVELPVSIVSLPV--------------------YHKHTFIVLY | 305 |
| OC43 | TQSIAPPTGVYELNGYTVQPIADVYRRKPNLPNCNIEAWLNDKSVPSPLNWERKTFSNCN | 361 |
| HKU1 | TKSLLPNTGVYDLSGFTVKPVATVHRRIPDLPDCDIDKWLNNFNVPSPLNWERKIFSNCN | 353 |
| MERS | YESFDVESGVYSVSSFEAKPSGSVVEQAEG-VECDFSPLLSG-TPPQVYNFKRLVFTNCN | 408 |
| SARS-CoV | VKSFEIDKGIYQTSNFRVVPSGDVVRFPNITNLCPFGEVENATKFPSVYAWERKKISNCV | 349 |
| SARS-CoV-2 | LKSFTVEKGIYQTSNFRVQPTESIVRFPNITNLCPFGEVENATRFASVYAWNRKRISNCV | 362 |
| Omicron | LKSFTVEKGIYQTSNFRVQPTESIVRFPNITNLCPFDEVFNATRFASVYAWNRKRISNCV | 359 |
| | .: *.*. . : : | |
| NL63 | --------------------ATASFGGSCYVCKPHQVNISLNG-------NTSVCVRTSH | 521 |
| 229E | -----------------VDFKPQSGGGKCFNCYPAGVNITLANFNET---KGPLCVDTSH | 345 |
| OC43 | FNMSSLMSFIQADSFTCNNIDAAKIYGMCFSSI------TIDKFAIPNGRKVDLQLGNLG | 415 |
| HKU1 | FNLSTLLRLVHTDSFSCNNFDESKIYGSCFKSI------VLDKFAIPNSRRSDLQLGSSG | 407 |
| MERS | YNLTKLLSLFSVNDFTCSQISPAAIASNCYSSL------ILDYFSYPLSMKSDLSVSSAG | 462 |
| SARS-CoV | ADYSVLYNSTFFSTFKCYGVSATKLNDLCFSNV------YADSFVVKGDDVRQIAPGQTG | 403 |
| SARS-CoV-2 | ADYSVLYNSASFSTFKCYGVSPTKLNDLCFTNV------YADSFVIRGDEVRQIAPGQTG | 416 |
| Omicron | ADYSVLYNLAPFFTFKCYGVSPTKLNDLCFTNV------YADSFVIRGDEVRQIAPGQTG | 413 |
| | . *: : | |
| NL63 | FSIRYIYNRVKSGSPGDSSWHIYL--KSGTCPFS-------FSKLN-------------- | 558 |
| 229E | FTTKYVAVYA-----NVGRWSASI--NTGNCPFS-------FGKVN-------------- | 377 |
| OC43 | YLQSFNYRIDTTATSCQLYYNLPAANVSVSR-FNPSTWNKRFGFIEDSVFKPRPAGVLTN | 474 |
| HKU1 | FLQSSNYKIDTTSSSCQLYYSLPAINVTINN-YNPSSWNRRYGFNNF---------NLSS | 457 |
| MERS | PISQFNYKQSFSNPTCLILATVPHNLTTITKPLKYSYINKCSRFLSD------------- | 509 |
| SARS-CoV | VIADYNYKLPDDFMGCVLAWNTRNIDATSTGNYNYKYRYLRHGKLRP------------- | 450 |
| SARS-CoV-2 | KIADYNYKLPDDFTGCVIAWNSNNLDSKVGGNYNYLYRLFRKSNLKP------------- | 463 |
| Omicron | NIADYNYKLPDDFTGCVIAWNSNKLDSKVSGNYNYLYRLFRKSNLKP------------- | 460 |
| | . . | |
| NL63 | NFQKFKTICFSTVEVPGSCNF--------------------------------------- | 579 |
| 229E | NFVKFGSVCFSLKDIPGGCAM--------------------------------------- | 398 |
| OC43 | HDVVYAQHCFKAPKNFCPCKLNGSCVGSGPGKNNGIGTCPAGTNYLTCD----------- | 523 |
| HKU1 | HSVVYSRYCFSVNNTFCPCAKPSFAS-SCKSHKPPSASCPIGTNYRSCESTTVLDHTDWC | 516 |
| MERS | DRTEVPQLVNA--NQYSPCVSI--------------------------VPSTVWEDGDYY | 541 |
| SARS-CoV | FERDISNVPFS--PDGKPCTP---------------------------PALNCY------ | 475 |
| SARS-CoV-2 | FERDISTEIYQ--AGSTPCNGV--------------------------EGFNCY------ | 489 |
| Omicron | FERDISTEIYQ--AGNKPCNGV--------------------------AGENCY------ | 486 |
| | * | |
| NL63 | -------PLEATWHYT--------------------SY-------------TIVGALYVT | 599 |
| 229E | -------PIVANWAYS--------------------KY-------------YTIGSLYVS | 418 |
| OC43 | -NLCTPDPITFTGTYKCPQTKSLVGIGEHCSGLAVKSDYCGG------NSCTCRPQAFLG | 576 |
| HKU1 | RCSCLPDPITAYDPRSCSQKKSLVGVGEHCAGFGVDEEKCGVLDGSYNVSCLCSTDAFLG | 576 |
| MERS | RKQLS--PLEGGGWLV-------------ASGSTVAMT----------------EQLQMG | 570 |
| SARS-CoV | ----W--PLNDYGFYT-------------TTGIGYQPY----------------RVVVLS | 500 |
| SARS-CoV-2 | ----F--PLQSYGFQP-------------TNGVGYQPY----------------RVVVLS | 514 |
| Omicron | ----F--PLRSYSFRP-------------TYGVGHQPY----------------RVVVLS | 511 |
| | *: : | |
| NL63 | WSEGNSITG--------------VPYPV-----SGIREFSNLVLNNCTKYNIYDYVGTGI | 640 |
| 229E | WSDGDGITG--------------VPQPV-----EGVSSFMNVTLDKCTKYNIYDVSGVGV | 459 |
| OC43 | WSADSCLQGDKCNIFANFILHDVNSGLTCST--DLQKANTDIILGVCVNYDLYGILGOGI | 634 |
| HKU1 | WSYDTCVSNNRCNIFSNFILNGINSGTTCSN--DLLQPNTEVFTDVCVDYDLYGITGQGI | 634 |
| MERS | FGIT--------------VQYGTDTNSVCPKLEFANDTKIASQLGNCVEYSLYGVSGRGV | 616 |
| SARS-CoV | FEL------------------LNAPATVCGP-----KLSTDLIKNQCVNFNFNGLTGTGV | 537 |
| SARS-CoV-2 | FEL------------------LHAPATVCGP-----KKSTNLVKNKCVNFNFNGLTGTGV | 551 |
| Omicron | FEL------------------LHAPATVCGP-----KKSTNLVKNKCVNFNFNGLKGTGV | 548 |
| | : . . *..:.: . * *: | |
| NL63 | IRSSNQSLAGG---ITYVSNSGNLL-GFKNVSTGNIFIVTPCNQPDQVAVYQQ------- | 689 |
| 229E | IRVSNDTFLNG---ITYTSTSGNLL-GFKDVTKGTIYSITPCNPPDQLVVYQQ------- | 508 |
| OC43 | FVEVNATYYNSWQNLLYD-SNGNLY-GFRDYIINRTFMIRSCYSGRVSAAFH--ANSSEP | 690 |
| HKU1 | FKEVSAVYYNSWQNLLYD-SNGNII-GFKDFVTNKTYNIFPCYAGRVSAAFH--QNASSL | 690 |
| MERS | FQNCTAVGV-RQQRFVYD-AYQNLVGYYSD--DGNYYCLRACVSVPVSVIYD--KETKTH | 670 |
| SARS-CoV | LTPSSKRFQ-PFQQFGRD-VSDFTD-SVRDPKTSEILDISPCSFGGVSVITPGTNASSEV | 594 |
| SARS-CoV-2 | LTESNKKFL-PFQQFGRD-IADTTD-AVRDPQTLEILDITPCSFGGVSVITPGTNTSNQV | 608 |
| Omicron | LTESNKKFL-PFQQFGRD-IADTTD-AVRDPQTLEILDITPCSFGGVSVITPGTNTSNQV | 605 |
| | : . : : : * . | |
| NL63 | -----SIIGAMTAVNESRYGLONLLQLP------NFYYVS----NGGNNCTT----AVMT | 730 |
| 229E | -----AVVGAMLSENFTSYGFSNVVELP------KFFYAS----NGTYNCTD----AVLT | 549 |
| OC43 | ALLFRNIKCNYVFNNSLTRQLQPI----------NYFDSYLGCVVNAYNSTAISVQTCDL | 740 |
| HKU1 | ALLYRNLKCSYVLNNIS---LTTQ----------PYFDSYLGCVFNADNLTDYSVSSCAL | 737 |
| MERS | ATLFGSVACEHISSTMSQYSRSTRSMLKRRDSTYGPLQTPVGCVLGLVNSS-LFVEDCKL | 729 |
| SARS-CoV | AVLYQDVNCTDVSTAIHADQLT--PAWRIYSTGNNVFQTQAGCLIGAEHVD--TSYECDI | 650 |
| SARS-CoV-2 | AVLYQDVNCTEVPVAIHADQLT--PTWRVYSTGSNVFQTRAGCLIGAEHVN--NSYECDI | 664 |
| Omicron | AVLYQGVNCTEVPVAIHADQLT--PTWRVYSTGSNVFQTRAGCLIGAEYVN--NSYECDI | 661 |
| | : . | |
| NL63 | YSNFGICADGSLI----PVRPRNSSDNG------------------I-SAIITANLSIPS | 767 |
| 229E | YSSFGVCADGSII----AVQPRNVSYDS------------------V-SAIVTANLSIPS | 586 |
| OC43 | TVGSGYCVDYSK-----NRRSRGAITTGYRFTNFEPFTVNS-VNDSLEPVGGLYEIQIPS | 794 |
| HKU1 | RMGSGFCVDYNSPSSSSSRRKRRSISASYRFVTFEPFNVSF-VNDSIESVGGLYEIKIPT | 796 |
| MERS | PLGQSLCALPDTPSTLTPRSVRSVPGEM-RLASI-AFNHPIQV-DQL--NSSYFKLSIPT | 784 |
| SARS-CoV | PIGAGICASYHTVSL-----LRSTSQKS-I---V-AYTMSLGADSSI--AYSNNTIAIPT | 698 |
| SARS-CoV-2 | PIGAGICASYQTQTN-SPRRARSVASQS-I---I-AYTMSLGAENSV--AYSNNSIAIPT | 716 |
| Omicron | PIGAGICASYQTQTK-SHRRARSVASQS-I---I-AYTMSLGAENSV--AYSNNSIAIPT | 713 |
| | . . *. * : : **: | |
| NL63 | NWTTSVQVEYLQITSTPIVVDCATYVCNGNPRCKNLLKQYTSACKTIEDALRLSAHLETN | 827 |
| 229E | NWTTSVQVEYLQITSTPIVVDCSTYVCNGNVRCVELLKQYTSACKTIEDALRNSARLESA | 646 |
| OC43 | EFTIGNMVEFIQTSSPKVTIDCAAFVCGDYAACKSQLVEYGSFCDNINAILTEVNELLDT | 854 |
| HKU1 | NFTIVGQEEFIQTNSPKVTIDCSLFVCSNYAACHDLLSEYGTFCDNINSILDEVNGLLDT | 856 |
| MERS | NFSFGVTQEYIQTTIQKVTVDCKQYVCNGFQKCEQLLREYGQFCSKINQALHGANLRQDD | 844 |
| SARS-CoV | NFSISITTEVMPVSMAKTSVDCNMYICGDSTECANLLLQYGSFCTQLNRALSGIAAEQDR | 758 |
| SARS-CoV-2 | NFTISVTTEILPVSMTKTSVDCTMYICGDSTECSNLLLQYGSFCTQLNRALTGIAVEQDK | 776 |
| Omicron | NFTISVTTEILPVSMTKTSVDCTMYICGDSTECSNLLLQYGSFCTQLKRALTGIAVEQDK | 773 |
| | ::: * : . :** ::*.. * . * :* * :: * | |
| NL63 | DVSSMLTFDSNAFSLANVTSF------GDYNLSSVLPQRNIRSSRIAGRSALEDLLFSKV | 881 |
| 229E | DVSEMLTFDKKAFTLANVSSF------GDYNLSSVIPSLPTSGSRVAGRSAIEDILFSKL | 700 |
| OC43 | TQLQVANSLMNGVTLSTKLKDGVNFNVDDINFSPVLGCLGSECSKASSRSAIEDLLFDKV | 914 |
| HKU1 | TQLHVADTLMQGVTLSSNLNTNLHFDVDNINFKSLVGCLGPHCGS-SSRSFFEDLLFDKV | 915 |
| MERS | SVRNLFASVKSSQSSPIIPGFG-----GDFNLTLLEP-VSISTGSRSARSAIEDLLFDKV | 898 |
| SARS-CoV | NTREVFAQVKQMYKTPTLKYFG-----GF-NFSQILP-D---PLKPTKRSFIEDLLFNKV | 808 |
| SARS-CoV-2 | NTQEVFAQVKQIYKTPPIKDFG-----GF-NFSQILP-D---PSKPSKRSFIEDLLFNKV | 826 |
| Omicron | NTQEVFAQVKQIYKTPPIKYFG-----GF-NFSQILP-D---PSKPSKRSFIEDLLFNKV | 823 |
| | : . . . *:. : : ** :**:**.*: | |
| NL63 | VTSGLGTVDVDYKSCT--KGLSIADLACAQYYNGIMVLPGVADAERMAMYTGSLIGGMVL | 939 |
| 229E | VTSGLGTVDADYKKCT--KGLSIADLACAQYYNGIMVLPGVADAERMAMYTGSLIGGIAL | 758 |
| OC43 | KLSDVGFVE-AYNNCT--GGAEIRDLICVQSYKGIKVLPPLLSENQISGYTLAATSASLF | 971 |
| HKU1 | KLSDVGFVE-AYNNCT--GGSEIRDLLCVQSFNGIKVLPPILSESQISGYTTAATVAAMF | 972 |
| MERS | TIADPGYMQ-GYDDCMQQGPASARDLICAQYVAGYKVLPPLMDVNMEAAYTSSLLGSIAG | 957 |
| SARS-CoV | TLADAGFMK-QYGECL--GDINARDLICAQKFNGLTVLPPLLTDDMIAAYTAALVSGTAT | 865 |
| SARS-CoV-2 | TLADAGFIK-QYGDCL--GDIAARDLICAQKFNGLTVLPPLLTDEMIAQYTSALLAGTIT | 883 |
| Omicron | TLADAGFIK-QYGDCL--GDIAARDLICAQKFKGLTVLPPLLTDEMIAQYTSALLAGTIT | 880 |
| | :. * :. * .* ** *.* * *** : . : ** : . | |
| NL63 | GGLTSA--AAIPFSLALQARLNYVALQTDVLQENQKILAASFNKAINNIVASFSSVND | 995 |
| 229E | GGLTSA----VSIPFSLAIQARLNYVALQTDVLQENQKILAASFNKAMINIVDAFTGVND | 814 |
| OC43 | PPWTAA----AGVPFYLNVQYRINGLGVTMDVLSQNQKLIANAFNNALYAIQEGFDATN- | 1026 |
| HKU1 | PPWSAA----AGIPFSLNVQYRINGLGVTMDVLNKNQKLIATAFNNALLSIQNGFSATN- | 1027 |
| MERS | VGWTAGLSSFAAIPFAQSIFYRLNGVGITQQVLSENQKLIANKFNQALGAMQTGFTTTN- | 1016 |
| SARS-CoV | AGWTFGAGAALQIPFAMQMAYRFNGIGVTQNVLYENQKQIANQFNKAISQIQESLTTTS- | 924 |
| SARS-CoV-2 | SGWTFGAGAALQIPFAMQMAYRFNGIGVTQNVLYENQKLIANQFNSAIGKIQDSLSSTA- | 942 |
| Omicron | SGWTFGAGAALQIPFAMQMAYRFNGIGVTQNVLYENQKLIANQFNSAIGKIQDSLSSTA- | 939 |
| | : . :** : *:* :.: :** :*** :* **.*: : .: . | |
| NL63 | AITQTAEAIHTVTIALNKIQDVVNQQGSALNHLTSQLRHNFQAISNSIQAIYDRLDSIQA | 1055 |
| 229E | AITQTSQALQTVATALNKIQDVVNQQGNSLNHLTSQLRONFQAISSSIQAIYDRLDTIQA | 874 |
| OC43 | -------------SALVKIQAVVNANAEALNNLLQQLSNRFGAISASLQEILSRLDALEA | 1073 |
| HKU1 | -------------SALAKIQSVVNSNAQALNSLLQQLFNKFGAISSSLQEILSRLDALEA | 1074 |
| MERS | -------------EAFHKVQDAVNNNAQALSKLASELSNTFGAISASIGDIIQRLDVLEQ | 1063 |
| SARS-CoV | -------------TALGKLQDVVNQNAQALNTLVKQLSSNFGAISSVLNDILSRLDKVEA | 971 |
| SARS-CoV-2 | -------------SALGKLQDVVNQNAQALNTLVKQLSSNFGAISSVLNDILSRLDKVEA | 989 |
| Omicron | -------------SALGKLQDVVNHNAQALNTLVKQLSSKFGAISSVLNDIFSRLDKVEA | 986 |
| | *: *:* .** :..:*. * .:* * *** : * .*** :: | |
| NL63 | DQQVDRLITGRLAALNAFVSQVLNKYTEVRGSRRLAQQKINECVKSQSNRYGFCGNGTHI | 1115 |
| 229E | DQQVDRLITGRLAALNVFVSHTLTKYTEVRASRQLAQQKVNECVKSQSKRYGFCGNGTHI | 934 |
| OC43 | EAQIDRLINGRLTALNAYVSQQLSDSTLVKFSAAQAMEKVNECVKSQSSRINFCGNGNHI | 1133 |
| HKU1 | QVQIDRLINGRLTALNAYVSQQLSDISLVKFGAALAMEKVNECVKSQSPRINFCGNGNHI | 1134 |
| MERS | DAQIDRLINGRLTTLNAFVAQQLVRSESAALSAQLAKDKVNECVKAQSKRSGFCGQGTHI | 1123 |
| SARS-CoV | EVQIDRLITGRLQSLQTYVTQQLIRAAEIRASANLAATKMSECVLGQSKRVDFCGKGYHL | 1031 |
| SARS-CoV-2 | EVQIDRLITGRLQSLQTYVTQQLIRAAEIRASANLAATKMSECVLGQSKRVDFCGKGYHL | 1049 |
| Omicron | EVQIDRLITGRLQSLQTYVTQQLIRAAEIRASANLAATKMSECVLGQSKRVDFCGKGYHL | 1046 |
| | : *:****.*** :*:.:*:: * . * *:.*** .** * .***:* *: | |
| NL63 | FSIVNSAPDGLLFLHTVLLPTDYKNVKAWSGICVDGIY--GYVLRQPNLVLY---SDNGV | 1170 |
| 229E | FSIVNAAPEGLVFLHTVLLPTQYKDVEAWSGLCVDGTN--GYVLRQPNLALY---KEGNY | 989 |
| OC43 | ISLVQNAPYGLYFIHFSYVPTKYVTARVSPGLCIAGDR--GIAPKSGYFVNV-----NNT | 1186 |
| HKU1 | LSLVQNAPYGLLFMHFSYKPISFKTVLVSPGLCISGDV--GIAPKQGYFIKH-----NDH | 1187 |
| MERS | VSFVVNAPNGLYFMHVGYYPSNHIEVVSAYGLCDAANPTNCIAPVNGYFIKTNNTRIVDE | 1183 |
| SARS-CoV | MSFPQAAPHGVVFLHVTYVPSQERNFTTAPAICHEGKA---YFPREGVFVFN-----GTS | 1083 |
| SARS-CoV-2 | MSFPQSAPHGVVFLHVTYVPAQEKNFTTAPAICHDGKA---HFPREGVFVSN-----GTH | 1101 |
| Omicron | MSFPQSAPHGVVFLHVTYVPAQEKNFTTAPAICHDGKA---HFPREGVFVSN-----GTH | 1098 |
| | .*: ** *: *:* * . .:* . . : | |
| NL63 | FRVTSRVMFQPRLPVLSDFVQIYNCNVTFVNISRVE--LHTVIPDYVDVNKTLQEFAQNL | 1228 |
| 229E | YRITSRIMFEPRIPTMADFVQIENCNVTFVNISRSE--LQTIVPEYIDVNKTLQELSYKL | 1047 |
| OC43 | WMYTGSGYYYPEPITENNVVVMSTCAVNYTKAPYVM--LNTSIPNLPDFKEELDQWFKNQ | 1244 |
| HKU1 | WMFTGSSYYYPEPISDKNVVFMNTCSVNFTKAPLVY--LNHSVPKLSDFESELSHWFKNQ | 1245 |
| MERS | WSYTGSSFYAPEPITSLNTKYVAPQVTYQN-ISTNLPPPLLGNSTGIDFQDELDEFFKNV | 1242 |
| SARS-CoV | WFITQRNFFSPQIITTDNTFVSGNCDVVIGIINNTVYDPLQ--PELDSFKEELDKYFKNH | 1141 |
| SARS-CoV-2 | WFVTQRNFYEPQIITTDNTFVSGNCDVVIGIVNNTVYDPLQ--PELDSFKEELDKYFKNH | 1159 |
| Omicron | WFVTQRNFYEPQIITTDNTFVSGNCDVVIGIVNNTVYDPLQ--PELDSFKEELDKYFKNH | 1156 |
| | : * : *. : . ..:. *.. : | |
| NL63 | PKYVKPNF--DLTPFNLTYLNLSSELKQLEAKTASLFQTTVELQGLIDQINSTYVDLKLL | 1286 |
| 229E | PNYTVPDL--VVEQYNQTILNLTSEISTLENKSAELNYTVQKLQTLIDNINSTLVDLKWL | 1105 |
| OC43 | TS-VAPDLSL--DYINVTFLDL--------------QVEMNRLQEAIKVLNQSYINLKDI | 1287 |
| HKU1 | TS-IAPNLTLNLHTINATFLDL--------------YYEMNLIQESIKSLNNSYINLKDI | 1290 |
| MERS | ST-SIPNFG-SLTQINTTLLDL--------------TYEMLSLQQVVKALNESYIDLKEL | 1286 |
| SARS-CoV | TS-PDVDLG-DISGINASVVNI--------------QKEIDRLNEVAKNLNESLIDLQEL | 1185 |
| SARS-CoV-2 | TS-PDVDLG-DISGINASVVNI--------------QKEIDRLNEVAKNLNESLIDLQEL | 1203 |
| Omicron | TS-PDVDLG-DISGINASVVNI--------------QKEIDRLNEVAKNLNESLIDLQEL | 1200 |
| | . :: * : ::: :: . :*.: ::*: : | |
| NL63 | NRFENYIKWPWWVWLIISVVFVVLLSLLVFCCLSTGCCGCCNCLTSSMRGCCDCGSTKLP | 1346 |
| 229E | NRVETYIKWPWWVWLCISVVLIFVVSMLLLCCCSTGCCGFFSCFASSIRGCCE--STKLP | 1163 |
| OC43 | GTYEYYVKWPWYVWLLICLAGVAMLVLLFFICCCTGCGTSCF-------K--KCGGCCDD | 1338 |
| HKU1 | GTYEMYVKWPWYVWLLISFSFIIFLVLLFFICCCTGCGSACF-------S--KCHNCCDE | 1341 |
| MERS | GNYTYYNKWPWYIWLGFIAGLVALALCVFFILCCTGCGTNCM-------GKLKCNRCCDR | 1339 |
| SARS-CoV | GKYEQYIKWPWYVWLGFIAGLIAIVMVTILLCCMTSCCSCLK-------GACSCGSCCKF | 1238 |
| SARS-CoV-2 | GKYEQYIKWPWYIWLGFIAGLIAIVMVTIMLCCMTSCCSCLK-------GCCSCGSCCKF | 1256 |
| Omicron | GKYEQYIKWPWYIWLGFIAGLIAIVMVTIMLCCMTSCCSCLK-------GCCSCGSCCKF | 1253 |
| | . * ****::** : : . .: *.* | |
| NL63 | YYEFEKVHVQ-------- (SEQ ID NO: 3) | 1356 |
| 229E | YYDVEKIHIQ-------- (SEQ ID NO: 4) | 1173 |
| OC43 | YTGYQELVIK-TSHDD-- (SEQ ID NO: 5) | 1353 |
| HKU1 | YGGHHDFVIK-TSHDD-- (SEQ ID NO: 6) | 1356 |
| MERS | YEEYDLEPHKVHVH---- (SEQ ID NO: 2) | 1353 |
| SARS-CoV | -DEDDSEPVLKGVKLHYT (SEQ ID NO: 7) | 1255 |
| SARS-CoV-2 | -DEDDSEPVLKGVKLHYT (SEQ ID NO: 8) | 1273 |
| Omicron | -DEDDSEPVLKGVKLHYT (SEQ ID NO: 1) | 1270 |

In some aspects, the amino acid corresponding to the amino acid at position 326 in SEQ ID NO:1 can be substituted to produce a variant coronavirus spike protein. In some aspects, the amino acid corresponding to the amino acid at position 326 in SEQ ID NO:1 can be substituted with a serine residue to produce a variant coronavirus spike protein. A substitution with a serine residue at 326 may be referred to herein as 326S.

In some aspects, the amino acid corresponding to the amino acid at position 364 in SEQ ID NO:1 can be substituted to produce a variant coronavirus spike protein. In some aspects, the amino acid corresponding to the amino acid at position 364 in SEQ ID NO:1 can be substituted with a phenylalanine residue to produce a variant coronavirus spike protein. A substitution with a phenylalanine residue at 364 may be referred to herein as 364F.

In some aspects, the amino acid corresponding to the amino acid at position 567 in SEQ ID NO:1 can be substituted to produce a variant coronavirus spike protein. In some aspects, the amino acid corresponding to the amino acid at position 567 in SEQ ID NO:1 can be substituted with a cysteine residue to produce a variant coronavirus spike protein. A substitution with a cysteine residue at 567 may be referred to herein as 567C.

In some aspects, the amino acid corresponding to the amino acid at position 611 in SEQ ID NO:1 can be substituted to produce a variant coronavirus spike protein. In some aspects, the amino acid corresponding to the amino acid at position 611 in SEQ ID NO:1 can be substituted with a glycine residue to produce a variant coronavirus spike protein. A substitution with a glycine residue at 611 may be referred to herein as 611G.

In some aspects, the amino acid corresponding to the amino acid at position 814 in SEQ ID NO:1 can be substituted to produce a variant coronavirus spike protein. In some aspects, the amino acid corresponding to the amino acid at position 814 in SEQ ID NO:1 can be substituted with a phenylalanine residue to produce a variant coronavirus spike protein. A substitution with a phenylalanine residue at 814 may be referred to herein as 814P.

In some aspects, the amino acid corresponding to the amino acid at position 840 in SEQ ID NO:1 can be substituted to produce a variant coronavirus spike protein. In some aspects, the amino acid corresponding to the amino acid at position 840 in SEQ ID NO:1 can be substituted with an asparagine residue to produce a variant coronavirus spike protein. A substitution with a asparagine residue at 840 may be referred to herein as 840N.

In some aspects, the amino acid corresponding to the amino acid at position 851 in SEQ ID NO:1 can be substituted to produce a variant coronavirus spike protein. In some aspects, the amino acid corresponding to the amino acid at position 851 in SEQ ID NO:1 can be substituted with a phenylalanine residue to produce a variant coronavirus spike protein. A substitution with a phenylalanine residue at 851 may be referred to herein as 851F.

In some aspects, the amino acid corresponding to the amino acid at position 889 in SEQ ID NO:1 can be substituted to produce a variant coronavirus spike protein. In some aspects, the amino acid corresponding to the amino acid at position 889 in SEQ ID NO:1 can be substituted with a proline residue to produce a variant coronavirus spike protein. A substitution with a proline residue at 889 may be referred to herein as 889P.

In some aspects, the amino acid corresponding to the amino acid at position 896 in SEQ ID NO:1 can be substituted to produce a variant coronavirus spike protein. In some aspects, the amino acid corresponding to the amino acid at position 896 in SEQ ID NO:1 can be substituted with a proline residue to produce a variant coronavirus spike protein. A substitution with a proline residue at 896 may be referred to herein as 896P.

In some aspects, the amino acid corresponding to the amino acid at position 939 in SEQ ID NO:1 can be substituted to produce a variant coronavirus spike protein. In some aspects, the amino acid corresponding to the amino acid at position 939 in SEQ ID NO:1 can be substituted with a proline residue to produce a variant coronavirus spike protein. A substitution with a proline residue at 939 may be referred to herein as 939P.

In some aspects, the amino acid corresponding to the amino acid at position 957 in SEQ ID NO:1 can be substituted to produce a variant coronavirus spike protein. In some aspects, the amino acid corresponding to the amino acid at position 957 in SEQ ID NO:1 can be substituted with a cysteine residue to produce a variant coronavirus spike protein. A substitution with a cysteine residue at 957 may be referred to herein as 957C.

In some aspects, the amino acid corresponding to the amino acid at position 977 in SEQ ID NO:1 can be substituted to produce a variant coronavirus spike protein. In some aspects, the amino acid corresponding to the amino acid at position 977 in SEQ ID NO:1 can be substituted with a cysteine residue to produce a variant coronavirus spike protein. A substitution with a cysteine residue at 977 may be referred to herein as 977C.

In some aspects, the amino acid corresponding to the amino acid at position 981 in SEQ ID NO:1 can be substituted to produce a variant coronavirus spike protein. In some aspects, the amino acid corresponding to the amino acid at position 981 in SEQ ID NO:1 can be substituted with a cysteine residue to produce a variant coronavirus spike protein. A substitution with a cysteine residue at 981 may be referred to herein as 981C.

In some aspects, the amino acid corresponding to the amino acid at position 982 in SEQ ID NO:1 can be substituted to produce a variant coronavirus spike protein. In some aspects, the amino acid corresponding to the amino acid at position 982 in SEQ ID NO:1 can be substituted with a proline residue to produce a variant coronavirus spike protein. A substitution with a proline residue at 982 may be referred to herein as 982P.

In some aspects, the amino acid corresponding to the amino acid at position 983 in SEQ ID NO:1 can be substituted to produce a variant coronavirus spike protein. In some aspects, the amino acid corresponding to the amino acid at position 983 in SEQ ID NO:1 can be substituted with a proline residue to produce a variant coronavirus spike protein. A substitution with a proline residue at 983 may be referred to herein as 983P.

In some aspects, the amino acid corresponding to the amino acid at position 984 in SEQ ID NO:1 can be substituted to produce a variant coronavirus spike protein. In some aspects, the amino acid corresponding to the amino acid at position 984 in SEQ ID NO:1 can be substituted with a proline residue to produce a variant coronavirus spike protein. A substitution with a proline residue at 983 may be referred to herein as 984P.

In some aspects, the amino acid corresponding to the amino acid at position 986 in SEQ ID NO:1 can be substituted to produce a variant coronavirus spike protein. In some aspects, the amino acid corresponding to the amino acid at position 986 in SEQ ID NO:1 can be substituted with a cysteine residue to produce a variant coronavirus spike protein. A substitution with a cysteine residue at 986 may be referred to herein as 986C.

In some aspects, the amino acid corresponding to the amino acid at position 989 in SEQ ID NO:1 can be substituted to produce a variant coronavirus spike protein. In some aspects, the amino acid corresponding to the amino acid at position 989 in SEQ ID NO:1 can be substituted with a cysteine residue to produce a variant coronavirus spike protein. A substitution with a cysteine residue at 989 may be referred to herein as 989C.

In some aspects, a variant spike protein has, has at least, or has at most 1, 2, 3, 4, 5, 6, 7, 8, 9, 10, 11, 12, 13, 14, 15, 16, 17, and/or 18 of the following modifications at positions 326, 364, 567, 611, 814, 840, 851, 889, 896, 939, 957, 977, 981, 982, 983, 984, 986, 989 as set forth in SARS-CoV-2 Omicron (BA1, previously B.1.1.529) spike protein, UniProt Accession Number UFO69279.1, or the corresponding amino acid in the spike protein of another coronavirus, wherein in some aspects the modification at the position or corresponding position 326 is a serine, 364 is a phenylalanine, 567 is a cysteine, 611 is a glycine, 814 is a proline, 840 is a asparagine, 851 is a phenylalanine, 889 is a proline, 896 is a proline, 939 is a proline, 957 is a cysteine, 977 is a cysteine, 981 is a cysteine, 982 is a proline, 983 is a proline, 984 is a proline, 986 is a cysteine, and 989 is a cysteine and wherein in further aspects the variant spike protein has at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to a SARS-CoV-2 Omicron (BA1, previously B.1.1.529) spike protein, UniProt Accession Number UFO69279.1. In some instances, the modifications described herein may be applied alone or in combination with any one or more additional modifications described herein to produce an isolated immunogenic polypeptide comprising a variant coronavirus spike protein that is a variant of a native coronavirus spike protein or fragment thereof. In some aspects, these modifications may (a) increase adoption by RBDs of the variant coronavirus spike proteins of the RBD-up conformation to expose more neutralization-sensitive epitopes on the spike protein, (b) decrease adoption by RBDs of the variant coronavirus spike proteins of the RBD-down conformation, (c) increase expression of the variant coronavirus spike protein compared to the native coronavirus spike protein, (d) increase adoption of a prefusion conformation, (e) decrease shedding of a S1 subunit of the variant coronavirus spike protein, and/or (f) improve localization of the variant coronavirus spike protein to a host cell membrane.

In some aspects, a variant spike protein has, has at least, or has at most 1, 2, 3, 4, 5, 6, 7, 8, 9, 10, 11, 12, 13, 14, 15, 16, 17, and/or 18 of the following modifications at positions 326, 364, 567, 611, 814, 840, 851, 889, 896, 939, 957, 977, 981, 982, 983, 984, 986, 989 as set forth in SARS-CoV-2 Omicron (BA1, previously B.1.1.529) spike protein, UniProt Accession Number UFO69279.1, or the corresponding amino acid in the spike protein of another coronavirus, wherein in some aspects the modification at the position or corresponding position 326 is to any amino acid except phenylalanine, 364 is any amino acid except valine, 567 is any amino acid except alanine, 611 is any amino acid except glycine, 814 is any amino acid except phenylalanine, 840 is any amino acid except aspartic acid, 851 is any amino acid except lysine, 889 is any amino acid except alanine, 896 is any amino acid except alanine, 939 is any amino acid except alanine, 957 is any amino acid except asparagine, 977 is any amino acid except isoleucine, 981 is any amino acid except leucine, 982 is any amino acid except aspartic acid, 983 is any amino acid except lysine, 984 is any amino acid except valine, 986 is any amino acid except alanine, and 989 is any amino acid except glutamine, and wherein in further aspects the variant spike protein has at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to a SARS-CoV-2 Omicron (BA1, previously B.1.1.529) spike protein, UniProt Accession Number UFO69279.1. In some instances, the modifications described herein may be applied alone or in combination with any one or more additional modifications described herein to produce an isolated immunogenic polypeptide comprising a variant coronavirus spike protein that is a variant of a native coronavirus spike protein or fragment thereof. In some aspects, these modifications may (a) increase adoption by RBDs of the variant coronavirus spike proteins of the RBD-up conformation to expose more neutralization-sensitive epitopes on the spike protein, (b) decrease adoption by RBDs of the variant coronavirus spike proteins of the RBD-down conformation, (c) increase expression of the variant coronavirus spike protein compared to the native coronavirus spike protein, (d) increase adoption of a prefusion conformation, (e) decrease shedding of a S1 subunit of the variant coronavirus spike protein, and/or (f) improve localization of the variant coronavirus spike protein to a host cell membrane.

In some instances, a variant spike protein is truncated as compared to SEQ ID NO: 1. The truncated variant in some instances can be transmembrane RBD trimer. The truncated variant in some instances can contain a RBD domain, and can further include a leader sequence, a transmembrane domain, and a trimerization domain. In some instances, the truncated variant does not contain an ER signal sequence. In some instances, the truncated variant contains a substitution of amino acid position corresponding to amino acid position 364 of SARS-CoV-2 Omicron (BA1, previously B.1.1.529) spike protein, UniProt Accession Number UFO69279.1. In some instances the substitution is to replace with a phenylalanine residue. In some instances, the leader sequence has 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity to a leader sequence of a SARS-CoV-2 Omicron (BA1, previously B.1.1.529) spike protein, UniProt Accession Number UFO69279.1. In some instances, the transmembrane domain has 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity to a transmembrane domain of a SARS-CoV-2 Omicron (BA1, previously B.1.1.529) spike protein, UniProt Accession Number UFO69279.1. In some instances, the trimerization domain has 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity to a trimerization domain of a SARS-CoV-2 Omicron (BA1, previously B.1.1.529) spike protein, UniProt Accession Number UFO69279.1. In some instances, the truncated variant has the amino acid sequence of RBDT3 as shown in Table 4.

TABLE 4
RBDT3 Sequence

MFVFLVLLPLVSSQCVNLTVRFPNITNLCPFGEVFNATRFASVYAWNRKR
ISNCVADYSFLYNSASFSTFKCYGVSPTKLNDLCFTNVYADSFVIRGDEV
RQIAPGQTGKIADYNYKLPDDFTGCVIAWNSNNLDSKVGGNYNYLYRLFR
KSNLKPFERDISTEIYQAGSTPCNGVEGFNCYFPLQSYGFQPTNGVGYQP
YRVVVLSFELLHAPATVCGPKGSPGSGSGSGYIPEAPRDGQAYVRKDGEW
VLLSTFLGSGSGSEQYIKWPWYIWLGFIAGLIAIVMVTIMLCCMTSCCSC
LKGCCSCGSCC

### C. Altered Amino Acids

As used herein, an “amino molecule” refers to any amino acid, amino acid derivative, or amino acid mimic as would be known to one of ordinary skill in the art. In certain aspects, the residues of the peptide or protein are sequential, without any non-amino molecule interrupting the sequence of amino molecule residues. In other aspects, the sequence may comprise one or more non-amino molecule moieties. In particular aspects, the sequence of residues of the proteinaceous molecule may be interrupted by one or more non-amino molecule moieties. Peptides and proteins include the twenty “natural” amino acids, and post-translational modifications thereof. However, in vitro peptide synthesis permits the use of modified and/or unusual amino acids.

Accordingly, the term “protein,” “peptide,” or “polypeptide” encompasses amino molecule sequences comprising at least one of the 20 common amino acids in naturally synthesized proteins, or at least one modified or unusual amino acid, including but not limited to those shown in the Table 5 below.

TABLE 5
Modified and Unusual Amino Acids

| Abbr. | Amino Acid | Abbr. | Amino Acid |
|---|---|---|---|
| Aad | 2-Aminoadipic acid | EtAsn | N-Ethylasparagine |
| Baad | 3-Aminoadipic acid | Hyl | Hydroxylysine |
| Bala | β-alanine, β-Amino-propionic acid | AHyl | allo-Hydroxylysine |
| Abu | 2-Aminobutyric acid | 3Hyp | 3-Hydroxyproline |
| 4Abu | 4-Aminobutyric acid, piperidinic acid | 4Hyp | 4-Hydroxyproline |
| Acp | 6-Aminocaproic acid | Ide | Isodesmosine |
| Ahe | 2-Aminoheptanoic acid | AIle | allo-Isoleucine |
| Aib | 2-Aminoisobutyric acid | MeGly | N-Methylglycine, sarcosine |
| Baib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

### VI. Obtaining Encoded Polypeptide Aspects

In some aspects, there are nucleic acid molecules encoding peptides of interest, e.g., antigens. These nucleic acids may be generated by methods known in the art.

### A. Expression

The nucleic acid molecules described herein may be used to express large quantities of the polypeptide of interest, such as an antigen, such as variant coronavirus spike protein.

### 1. Nucleic Acid Synthesis

In some aspects, contemplated are isolated nucleic acid molecules comprising a nucleic acid molecule encoding a polypeptide of the desired sequence or a portion thereof (e.g., a fragment containing one or more polypeptides, or antigens). In some aspects, nucleic acid molecules comprising nucleic acid molecules may encode antigens, fusion proteins, modified antibodies, antibody fragments, and probes thereof. In addition to control sequences that govern transcription and translation, the nucleic acid molecules may contain nucleic acid sequences that serve other functions as well.

In some aspects, the nucleic acid molecule is an analog and may include modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

Modified oligonucleotides and oligonucleotide analogs may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified intemucleoside linkages are intended to replace naturally-occurring phosphodiester-5′-methylene linkages with four atom linking groups to confer nuclease resistance and enhanced cellular uptake to the resulting compound.

Modifications may be achieved using solid supports which may be manually manipulated or used in conjunction with a nucleic acid synthesizer using methodology commonly known to those skilled in nucleic acid synthesizer art. Generally, the procedure involves functionalizing the sugar moieties of two nucleosides which will be adjacent to one another in the selected sequence. In a 5′ to 3′ sense, an “upstream” synthon such as structure H is modified at its terminal 3′ site, while a “downstream” synthon such as structure H1 is modified at its terminal 5′ site.

Oligonucleosides linked by hydrazines, hydroxylarnines, and other linking groups can be protected by a dimethoxytrityl group at the 5′-hydroxyl and activated for coupling at the 3′-hydroxyl with cyanoethyldiisopropyl-phosphite moieties. These compounds can be inserted into any desired sequence by standard, solid phase, automated nucleic acid synthesis techniques. One of the most popular processes is the phosphoramidite technique. Oligonucleotides containing a uniform backbone linkage can be synthesized by use of CPG-solid support and standard nucleic acid synthesizing machines such as Applied Biosystems Inc. 380B and 394 and Milligen/Biosearch 7500 and 8800s. The initial nucleotide (number 1 at the 3′-terminus) is attached to a solid support such as controlled pore glass. In sequence specific order, each new nucleotide is attached either by manual manipulation or by the automated synthesizer system.

Free amino groups can be alkylated with, for example, acetone and sodium cyanoboro hydride in acetic acid. The alkylation step can be used to introduce other, useful, functional molecules on the macromolecule. Such useful functional molecules include but are not limited to reporter molecules, cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference, and in other of the above-referenced patent applications.

Solid supports may include any of those known in the art for polynucleotide synthesis, including controlled pore glass (CPG), oxalyl controlled pore glass, TentaGel Support—an aminopolyethyleneglycol derivatized support or Poros—a copolymer of polystyrene/divinylbenzene. Attachment and cleavage of nucleotides and oligonucleotides can be effected via standard procedures. As used herein, the term solid support further includes any linkers (e.g., long chain alkyl amines and succinyl residues) used to bind a growing oligonucleoside to a stationary phase such as CPG. In some aspects, the oligonucleotide may be further defined as having one or more locked nucleotides, ethylene bridged nucleotides, peptide nucleic acids, or a 5′(E)-vinyl-phosphonate (VP) modification. In some aspects, the oligonucleotides has one or more phosphorothioated nucleic acid bases.

### 2. Expression Systems

Numerous expression systems exist that comprise at least a part or all of the proteins, peptides, or nucleic acid molecules discussed above. Prokaryote- and/or eukaryote-based systems or cell free systems can be employed for use with an aspect to produce proteins, peptides, nucleic acid sequences, or their cognate polypeptides, proteins and peptides. Commercially and widely available systems include but are not limited to bacterial, mammalian, yeast, insect cell, and cell free systems. Different host cells have characteristic and specific mechanisms for the post-translational processing and modification of proteins. Appropriate cell lines, host systems, or expression systems can be chosen to ensure the correct modification and processing of the nucleic acid or polypeptide(s) expressed. Those skilled in the art are able to express a vector to produce a nucleic acid sequence or its cognate polypeptide, protein, or peptide using an appropriate expression system.

In some aspects, immunogenic variant coronavirus spike protein constructs and/or nucleic acids encoding the immunogenic variant coronavirus spike protein constructs of the present disclosure are achieved by operably linking a nucleic acid encoding the immunogenic variant coronavirus spike protein constructs to a promoter, and incorporating the construct into an expression vector, which is taken up and expressed by cells. The vectors can be suitable for replication and, in some cases, integration in eukaryotes. Typical vectors contain transcription and translation terminators, initiation sequences, and promoters useful for regulation of the expression of the desired nucleic acid sequence. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

In certain aspects the expression vector may be provided to a cell in the form of a viral vector. Viral vector technology is well known in the art and is described, for example, in Sambrook et al. (2001) Molecular Cloning: A Laboratory Manual, Cold Spring Harbor Laboratory, New York), and in other virology and molecular biology manuals.

A number of viral based systems have been developed for gene transfer into mammalian cells. Viruses that are useful as vectors include, but are not limited to, retroviruses, adenoviruses, adeno-associated viruses, herpes viruses, and lentiviruses (including self-inactivating lentivirus vectors). For example, adenoviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. Thus, in some aspects, the nucleic acid encoding immunogenic polypeptide constructs of the present disclosure is introduced into cells using a recombinant vector such as a viral vector including, for example, a lentivirus, a retrovirus, gamma-retroviruses, an adeno-associated virus (AAV), a herpesvirus, or an adenovirus.

One of skill in the art would be well equipped to construct a vector comprising one or more polynucleotide sequences of interest through standard recombinant techniques (see, for example, Maniatis et al., 1988 and Ausubel et al., 1994, both specifically incorporated by reference herein in their entirety).

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also might include markers, such as detectable and/or selection markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

Eukaryotic expression cassettes included in the vectors particularly contain (in a 5′-to-3′ direction) regulatory elements including a eukaryotic transcriptional promoter operably linked to a protein-coding sequence, splice signals including intervening sequences, a transcriptional termination/polyadenylation sequence, post-transcriptional regulatory elements, and origins of replication.

### 3. Methods of Nucleic Acid Delivery

Suitable methods for nucleic acid delivery to effect expression of compositions are anticipated to include virtually any method by which a particular nucleic acid (e.g., DNA) can be introduced into a cell, a tissue or an organism, as described herein or as would be known to one of ordinary skill in the art. Such methods may include, but are not limited to, direct delivery of nucleic acids such as by injection (U.S. Pat. No. 5,994,624,5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

One illustrative delivery vehicle is a lipid and/or a liposome. The use of lipid formulations is contemplated for the introduction of the nucleic acids into a host cell (in vitro, ex vivo, or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

In a certain aspect, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The amount of liposomes used may vary upon the nature of the liposome as well as the cell used, for example, about 5 to about 20 g vector DNA per 1 to 10 million of cells may be contemplated.

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain aspects, a liposome may be complexed with a hemagglutinating virus (HVJ). This has been shown to facilitate fusion with the cell membrane and promote cell entry of liposome-encapsulated DNA (Kaneda et al., 1989). In other aspects, a liposome may be complexed or employed in conjunction with nuclear non-histone chromosomal proteins (HMG-1) (Kato et al., 1991). In yet further aspects, a liposome may be complexed or employed in conjunction with both HVJ and HMG-1. In other aspects, a delivery vehicle may comprise a ligand and a liposome.

In various aspects lipids suitable for use can be obtained from commercial sources. For example, lipofectamine can be obtained from Thermo Fisher Scientific, Waltham, Mass.; dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform can be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al. (1991) Glycobiology 5: 505-510). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

In certain aspects, a nucleic acid is introduced into a cell via electroporation. Electroporation involves the exposure of a suspension of cells and DNA to a high-voltage electric discharge. Recipient cells can be made more susceptible to transformation by mechanical wounding. Also the amount of vectors used may vary upon the nature of the cells used, for example, about 5 to about 20 g vector DNA per 1 to 10 million of cells may be contemplated.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

In other aspects, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

In another aspect, a nucleic acid is delivered into a cell using DEAE-dextran followed by polyethylene glycol. In this manner, reporter plasmids were introduced into mouse myeloma and erythroleukemia cells (Gopal, 1985).

### 4. Host Cells

In another aspect, contemplated are the use of host cells into which a nucleic acid molecule has been introduced. Nucleic acids can be transfected into cells according to a variety of methods known in the art. Nucleic acids can be introduced into prokaryotic or eukaryotic cells via conventional transformation or transfection techniques. Some nucleic acids may employ control sequences that allow it to be replicated and/or expressed in both prokaryotic and eukaryotic cells. In certain aspects, the polypeptide of interest expression construct or nucleic acid replicase can be placed under control of a promoter that is linked to T-cell activation, such as one that is controlled by NFAT-1 or NF-κB, both of which are transcription factors that can be activated upon T-cell activation. Control of expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a nucleic acid molecule. Also understood and known are techniques and conditions that would allow large-scale production of nucleic acid molecules, as well as production of the nucleic acids encoded by vectors and their cognate polypeptides, proteins, or peptides.

For transfection of mammalian cells, it is known, depending upon the nucleic acid and transfection technique used, only a small fraction of cells may integrate the foreign nucleic acid into their cells. Regardless of the method used to introduce exogenous nucleic acids into a host cell, in order to identify and select these integrants, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

In certain aspects, cells containing an exogenous nucleic acid may be identified in vitro or in vivo by including a marker in the expression vector or the exogenous nucleic acid. Such markers would confer an identifiable change to the cell permitting easy identification of cells containing the expression vector. Generally, a selection marker may be one that confers a property that allows for selection. A positive selection marker may be one in which the presence of the marker allows for its selection, while a negative selection marker is one in which its presence prevents its selection. An example of a positive selection marker is a drug resistance marker.

In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

Selectable markers may include a type of reporter gene used in laboratory microbiology, molecular biology, and genetic engineering to indicate the success of a transfection or other procedure meant to introduce foreign DNA into a cell. Selectable markers are often antibiotic resistance genes; cells that have been subjected to a procedure to introduce foreign DNA are grown on a medium containing an antibiotic, and those cells that can grow have successfully taken up and expressed the introduced genetic material. Examples of selectable markers include: the Abier gene or Neo gene from Tn5, which confers antibiotic resistance to geneticin.

A screenable marker may comprise a reporter gene, which allows the researcher to distinguish between wanted and unwanted cells. Certain aspects of the present invention utilize reporter genes to indicate specific cell lineages. For example, the reporter gene can be located within expression elements and under the control of the ventricular- or atrial-selective regulatory elements normally associated with the coding region of a ventricular- or atrial-selective gene for simultaneous expression. A reporter allows the cells of a specific lineage to be isolated without placing them under drug or other selective pressures or otherwise risking cell viability.

Examples of such reporters include genes encoding cell surface proteins (e.g., CD4, HA epitope), fluorescent proteins, antigenic determinants and enzymes (e.g., β-galactosidase). The vector containing cells may be isolated, e.g., by FACS using fluorescently-tagged antibodies to the cell surface protein or substrates that can be converted to fluorescent products by a vector encoded enzyme.

In specific aspects, the reporter gene is a fluorescent protein. A broad range of fluorescent protein genetic variants have been developed that feature fluorescence emission spectral profiles spanning almost the entire visible light spectrum (see Table 1 for non-limiting examples). Mutagenesis efforts in the original Aequorea victoria jellyfish green fluorescent protein have resulted in new fluorescent probes that range in color from blue to yellow, and are some of the most widely used in vivo reporter molecules in biological research. Longer wavelength fluorescent proteins, emitting in the orange and red spectral regions, have been developed from the marine anemone, Discosoma striata, and reef corals belonging to the class Anthozoa. Still other species have been mined to produce similar proteins having cyan, green, yellow, orange, and deep red fluorescence emission. Developmental research efforts are ongoing to improve the brightness and stability of fluorescent proteins, thus improving their overall usefulness.

In particular aspects, the cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain aspects, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). In specific aspects, one or more of these may be explicitly excluded.

In some aspects, the medium further comprises vitamins. In some aspects, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some aspects, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some aspects, the vitamins include or consist essentially of biotin, DL alpha tocopherol acetate, DL alpha-tocopherol, vitamin A, or combinations or salts thereof. In some aspects, the medium further comprises proteins. In some aspects, the proteins comprise albumin or bovine serum albumin, a fraction of BSA, catalase, insulin, transferrin, superoxide dismutase, or combinations thereof. In some aspects, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some aspects, the medium comprises one or more of the following: a B-27® supplement, xeno-free B-27® supplement, GS21TM supplement, or combinations thereof. In some aspects, the medium comprises or further comprises amino acids, monosaccharides, inorganic ions. In some aspects, the amino acids comprise arginine, cystine, isoleucine, leucine, lysine, methionine, glutamine, phenylalanine, threonine, tryptophan, histidine, tyrosine, or valine, or combinations thereof. In some aspects, the inorganic ions comprise sodium, potassium, calcium, magnesium, nitrogen, or phosphorus, or combinations or salts thereof. In some aspects, the medium further comprises one or more of the following: molybdenum, vanadium, iron, zinc, selenium, copper, or manganese, or combinations thereof. In certain aspects, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21TM supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific aspects, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific aspects, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/mL, μg/mL, mg/mL, or any range derivable therein.

In specific aspects, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular aspects the cells are in a formulated cell suspension that is stable at room temperature for 1, 2, 3, or 4 hours or more from time of thawing.

### B. Isolation

The nucleic acid molecules disclosed herein may be obtained from any source that produces nucleic acids. Methods of isolating nucleic acids are well known in the art.

### VII. Immune Response and Assays

As discussed herein, the disclosure concerns evoking or inducing an immune response in a subject against a coronavirus protein, e.g., a native or variant coronavirus spike protein. In one aspect, the immune response can protect against or treat a subject having, suspected of having, or at risk of developing an infection or related disease, particularly those related to coronaviruses. One use of the immunogenic compositions of the disclosure is to prevent coronavirus infections by inoculating a subject.

### A. Immunoassays

The present disclosure includes the implementation of serological assays to evaluate whether and to what extent an immune response is induced or evoked by compositions of the disclosure. There are many types of immunoassays that can be implemented. Immunoassays encompassed by the present disclosure include, but are not limited to, those described in U.S. Pat. No. 4,367,110 (double monoclonal antibody sandwich assay) and U.S. Pat. No. 4,452,901 (western blot). Other assays include immunoprecipitation of labeled ligands and immunocytochemistry, both in vitro and in vivo.

Immunoassays generally are binding assays. Certain preferred immunoassays are the various types of enzyme linked immunosorbent assays (ELISAs) and radioimmunoassays (RIA) known in the art. Immunohistochemical detection using tissue sections is also particularly useful. In one example, antibodies or antigens are immobilized on a selected surface, such as a well in a polystyrene microtiter plate, dipstick, or column support. Then, a test composition suspected of containing the desired antigen or antibody, such as a clinical sample, is added to the wells. After binding and washing to remove non-specifically bound immune complexes, the bound antigen or antibody may be detected. Detection is generally achieved by the addition of another antibody, specific for the desired antigen or antibody, that is linked to a detectable label. This type of ELISA is known as a “sandwich ELISA.” Detection also may be achieved by the addition of a second antibody specific for the desired antigen, followed by the addition of a third antibody that has binding affinity for the second antibody, with the third antibody being linked to a detectable label.

Competition ELISAs are also possible implementations in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal. Irrespective of the format employed, ELISAs have certain features in common, such as coating, incubating or binding, washing to remove non-specifically bound species, and detecting the bound immune complexes.

Antigen or antibodies may also be linked to a solid support, such as in the form of plate, beads, dipstick, membrane, or column matrix, and the sample to be analyzed is applied to the immobilized antigen or antibody. In coating a plate with either antigen or antibody, one will generally incubate the wells of the plate with a solution of the antigen or antibody, either overnight or for a specified period. The wells of the plate will then be washed to remove incompletely-adsorbed material. Any remaining available surfaces of the wells are then “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

### B. Diagnosis of Coronavirus Infection

In addition to the use of proteins, polypeptides, and/or peptides to treat, prevent, or reduce the severity of illness from infection as described above, the present disclosure contemplates the use of these polypeptides, proteins, and/or peptides in a variety of ways, including the detection of the presence of coronavirus to diagnose an infection. In accordance with the disclosure, a preferred method of detecting the presence of infections involves the steps of obtaining a sample suspected of being infected by one or more coronavirus strains, such as a sample taken from an individual, for example, from one's blood, saliva, tissues, bone, muscle, cartilage, or skin. Following isolation of the sample, diagnostic assays utilizing the polypeptides, proteins, and/or peptides of the present disclosure may be carried out to detect the presence of coronavirus, and such assay techniques for determining such presence in a sample are well known to those skilled in the art and include methods such as radioimmunoassay, western blot analysis and ELISA assays.

In general, in accordance with the disclosure, a method of diagnosing an infection is contemplated wherein a sample suspected of being infected with coronavirus has added to it the polypeptide, protein, or peptide, in accordance with the present disclosure, and coronaviruses are indicated by antibody binding to the polypeptides, proteins, and/or peptides, or polypeptides, proteins, and/or peptides binding to the antibodies in the sample.

Accordingly, polypeptides, proteins, and/or peptides in accordance with the disclosure may be used for to treat, prevent, or reduce the severity of illness from infection due to coronavirus infection (i.e., active or passive immunization) or for use as research tools.

Any of the above described polypeptides, proteins, and/or peptides may be labeled directly with a detectable label for identification and quantification of coronavirus. Labels for use in immunoassays are generally known to those skilled in the art and include enzymes, radioisotopes, and fluorescent, luminescent and chromogenic substances, including colored particles such as colloidal gold or latex beads. Suitable immunoassays include enzyme-linked immunosorbent assays (ELISA).

### C. Protective Immunity

In some aspects of the disclosure, proteinaceous compositions confer protective immunity to a subject. Protective immunity refers to a body's ability to mount a specific immune response that protects the subject from developing a particular disease or condition that involves the agent against which there is an immune response. An immunogenically effective amount is capable of conferring protective immunity to the subject.

As used herein in the specification and in the claims section that follows, the term polypeptide or peptide refer to a stretch of amino acids covalently linked there amongst via peptide bonds. Different polypeptides have different functionalities according to the present invention. While according to one aspect, a polypeptide is derived from an immunogen designed to induce an active immune response in a recipient, according to another aspect of the invention, a polypeptide is derived from an antibody which results following the elicitation of an active immune response in, for example, an animal, and which can serve to induce a passive immune response in the recipient. In both cases, however, the polypeptide is encoded by a polynucleotide according to any possible codon usage.

As used herein the phrase “immune response” or its equivalent “immunological response” refers to the development of a humoral (antibody mediated), cellular (mediated by antigen-specific T cells or their secretion products) or both humoral and cellular response directed against a protein, peptide, carbohydrate, or polypeptide of the invention in a recipient patient. Such a response can be an active response induced by administration of immunogen or a passive response induced by administration of antibody, antibody containing material, or primed T-cells. A cellular immune response is elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity. As used herein “active immunity” refers to any immunity conferred upon a subject by administration of an antigen.

As used herein “passive immunity” refers to any immunity conferred upon a subject without administration of an antigen to the subject. “Passive immunity” therefore includes, but is not limited to, administration of activated immune effectors including cellular mediators or protein mediators (e.g., monoclonal and/or polyclonal antibodies) of an immune response. A monoclonal or polyclonal antibody composition may be used in passive immunization to treat, prevent, or reduce the severity of illness caused by infection by organisms that carry the antigen recognized by the antibody. An antibody composition may include antibodies that bind to a variety of antigens that may in turn be associated with various organisms. The antibody component can be a polyclonal antiserum. In certain aspects the antibody or antibodies are affinity purified from an animal or second subject that has been challenged with an antigen(s). Alternatively, an antibody mixture may be used, which is a mixture of monoclonal and/or polyclonal antibodies to antigens present in the same, related, or different microbes or organisms, such as viruses, including but not limited to coronaviruses.

Passive immunity may be imparted to a patient or subject by administering to the patient immunoglobulins (Ig) and/or other immune factors obtained from a donor or other non-patient source having a known immunoreactivity. In other aspects, an immunogenic composition of the present disclosure can be administered to a subject who then acts as a source or donor for globulin, produced in response to challenge with the immunogenic composition (“hyperimmune globulin”), that contains antibodies directed against a coronavirus or other organism. A subject thus treated would donate plasma from which hyperimmune globulin would then be obtained, via conventional plasma-fractionation methodology, and administered to another subject in order to impart resistance against or to treat coronavirus infection.

For purposes of this specification and the accompanying claims the terms “epitope” and “antigenic determinant” are used interchangeably to refer to a site on an antigen to which B and/or T cells respond or recognize. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols (1996). Antibodies that recognize the same epitope can be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke et al., 1994), by antigen-dependent killing (cytotoxic T lymphocyte assay, Tigges et al., 1996) or by cytokine secretion.

The presence of a cell-mediated immunological response can be determined by proliferation assays (CD4 (+) T cells) or CTL (cytotoxic T lymphocyte) assays. The relative contributions of humoral and cellular responses to the protective or therapeutic effect of an immunogen can be distinguished by separately isolating IgG and T-cells from an immunized syngeneic animal and measuring protective or therapeutic effect in a second subject.

As used herein, the terms “antibody” or “immunoglobulin” are used interchangeably and refer to any of several classes of structurally related proteins that function as part of the immune response of an animal or recipient, which proteins include IgG, IgD, IgE, IgA, IgM and related proteins. Under normal physiological conditions antibodies are found in plasma and other body fluids and in the membrane of certain cells and are produced by lymphocytes of the type denoted B cells or their functional equivalent.

As used herein the terms “immunogenic agent” or “immunogen” or “antigen” are used interchangeably to describe a molecule capable of inducing an immunological response against itself on administration to a recipient, either alone, in conjunction with an adjuvant, or presented on a display vehicle.

### VIII. Compositions

In one aspect, the disclosure relates to an immunogenic composition for administration to a host. In some aspects, the host is a human. In other aspects, the host is a non-human.

In some aspects, the composition comprises immunogenic polypeptide construct. In some aspects, the immunogenic polypeptide construct of the composition is an isolated immunogenic polypeptide comprising a variant coronavirus spike protein that is a variant of a native coronavirus spike protein or fragment thereof and/or a nucleic acid encoding the isolated immunogenic polypeptide comprising a variant coronavirus spike protein that is a variant of a native coronavirus spike protein or fragment thereof. In some aspects, the composition comprises a variant coronavirus spike protein construct and/or a nucleic acid encoding the modified variant coronavirus spike protein construct. The variant coronavirus spike protein construct can differ from a native, unmodified coronavirus spike protein construct at one or more amino acids. In some aspects, the variant coronavirus spike protein construct has at least about 50% amino acid sequence identity with the native, unmodified coronavirus spike protein construct.

### A. Vaccines

In some instances, the compositions described herein are immunogenic compositions. In some instances, the compositions described herein include at least one isolated nucleic acid or polypeptide molecule as described herein. In some instances, the compositions described herein are vaccines. In specific aspects, the immunogenic compositions comprise nucleic acids, and the immunogenic compositions are nucleic acid vaccines. In other aspects, the immunogenic compositions comprise DNA, and vaccines are DNA vaccines. In yet other aspects, the immunogenic compositions comprise a polypeptide, and vaccines are polypeptide vaccines. Conditions and/or diseases that can be treated with the nucleic acid and/or peptide or polypeptide compositions include, but are not limited to, those caused and/or impacted by infection, cancer, rare diseases, and other diseases or conditions caused by overproduction, underproduction, or improper production of protein or nucleic acids.

In some aspects, the composition is substantially free of one or more impurities or contaminants and, for instance, includes nucleic acid or polypeptide molecules that are equal to any one of, at least any one of, at most any one of, or between any two of 90%, 91%, 92%, 93%, 94%, 95%, 96%, 97%, 98%, or 99% pure; at least 98% pure, or at least 99% pure.

The present invention includes methods for preventing or ameliorating coronavirus infections. As such, the invention contemplates vaccines for use in both active and passive immunization aspects. Immunogenic compositions, proposed to be suitable for use as a vaccine, may be prepared from native or variant coronavirus polypeptide(s), such as a native or variant coronavirus spike proteins. In other aspects coronavirus spike proteins can be used in combination with other secreted virulence proteins, surface proteins, or immunogenic fragments thereof. In certain aspects, antigenic material is extensively dialyzed to remove undesired small molecular weight molecules and/or lyophilized for more ready formulation into a desired vehicle.

The preparation of vaccines that contain nucleic acid and/or peptide or polypeptide as active ingredients is generally well understood in the art, as exemplified by U.S. Pat. Nos. 4,608,251; 4,601,903; 4,599,231; 4,599,230; 4,596,792; and 4,578,770, all of which are incorporated herein by reference. Typically, such vaccines are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific aspects, vaccines are formulated with a combination of substances, as described in U.S. Pat. Nos. 6,793,923 and 6,733,754, which are incorporated herein by reference.

Vaccines may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

The polypeptides and polypeptide-encoding nucleic acid constructs may be formulated into a vaccine as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like.

Typically, vaccines are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms of active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application within a solid physiologically acceptable base or in a physiologically acceptable dispersion, parenterally, by injection and the like. The dosage of the vaccine will depend on the route of administration and will vary according to the size and health of the subject.

In certain instances, it will be desirable to have multiple administrations of the vaccine, e.g., 2, 3, 4, 5, 6 or more administrations. The vaccinations can be at 1, 2, 3, 4, 5, 6, 7, 8, to 5, 6, 7, 8, 9, 10, 11, 12 twelve week intervals, including all ranges there between. Periodic boosters at intervals of 1-5 years will be desirable to maintain protective levels of the antibodies. The course of the immunization may be followed by assays for antibodies against the antigens, as described in U.S. Pat. Nos. 3,791,932; 4,174,384 and 3,949,064.

### 1. Carriers

A given composition may vary in its immunogenicity. It is often necessary therefore to boost the host immune system, as may be achieved by coupling a peptide or polypeptide to a carrier. Exemplary carriers are keyhole limpet hemocyanin (KLH) and bovine serum albumin (BSA). Other albumins such as ovalbumin, mouse serum albumin, or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde, and bis-biazotized benzidine.

### 2. Adjuvants

The immunogenicity of polypeptide or peptide compositions can be enhanced by the use of non-specific stimulators of the immune response, known as adjuvants. Suitable adjuvants include all acceptable immunostimulatory compounds, such as cytokines, toxins, or synthetic compositions. A number of adjuvants can be used to enhance an antibody response against a variant SpA polypeptide or coagulase, or any other bacterial protein or combination contemplated herein. Adjuvants can (1) trap the antigen in the body to cause a slow release; (2) attract cells involved in the immune response to the site of administration; (3) induce proliferation or activation of immune system cells; or (4) improve the spread of the antigen throughout the subject's body.

Adjuvants include, but are not limited to, oil-in-water emulsions, water-in-oil emulsions, mineral salts, polynucleotides, and natural substances. Specific adjuvants that may be used include IL-1, IL-2, IL-4, IL-7, IL-12, □-interferon, GMCSP, BCG, aluminum salts, such as aluminum hydroxide or other aluminum compound, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM), and cell wall skeleton (CWS) in a 2% squalene/Tween 80 emulsion. MHC antigens may even be used. Others adjuvants or methods are exemplified in U.S. Pat. Nos. 6,814,971, 5,084,269, 6,656,462, each of which is incorporated herein by reference).

Various methods of achieving adjuvant affect for the vaccine includes use of agents such as aluminum hydroxide or phosphate (alum), commonly used as about 0.05 to about 0.1% solution in phosphate buffered saline, admixture with synthetic polymers of sugars (Carbopol®) used as an about 0.25% solution, aggregation of the protein in the vaccine by heat treatment with temperatures ranging between about 70° to about 101° C. for a 30-second to 2-minute period, respectively. Aggregation by reactivating with pepsin-treated (Fab) antibodies to albumin; mixture with bacterial cells (e.g., C. parvum), endotoxins or lipopolysaccharide components of Gram-negative bacteria; emulsion in physiologically acceptable oil vehicles (e.g., mannide mono-oleate (Aracel A)); or emulsion with a 20% solution of a perfluorocarbon (Fluosol-DA®) used as a block substitute may also be employed to produce an adjuvant effect.

Examples of and often preferred adjuvants include complete Freund's adjuvant (a non-specific stimulator of the immune response containing killed Mycobacterium tuberculosis), incomplete Freund's adjuvants, and aluminum hydroxide.

In some aspects, it is preferred that the adjuvant be selected to be a preferential inducer of either a Th1 or a Th2 type of response. High levels of Th1-type cytokines tend to favor the induction of cell mediated immune responses to a given antigen, while high levels of Th2-type cytokines tend to favor the induction of humoral immune responses to the antigen.

The distinction of Th1 and Th2-type immune response is not absolute. In reality an individual will support an immune response which is described as being predominantly Th1 or predominantly Th2. However, it is often convenient to consider the families of cytokines in terms of that described in murine CD4+ T cell clones by Mosmann and Coffman (Mosmann, and Coffman, 1989). Traditionally, Th1-type responses are associated with the production of the INF-γ and IL-2 cytokines by T-lymphocytes. Other cytokines often directly associated with the induction of Th1-type immune responses are not produced by T-cells, such as IL-12. In contrast, Th2-type responses are associated with the secretion of IL-4, IL-5, IL-6, IL-10.

In addition to adjuvants, it may be desirable to co-administer biologic response modifiers (BRM) to enhance immune responses. BRMs have been shown to upregulate T cell immunity or downregulate suppresser cell activity. Such BRMs include, but are not limited to, Cimetidine (CIM; 1200 mg/d) (Smith/Kline, PA); or low-dose Cyclophosphamide (CYP; 300 mg/m²) (Johnson/Mead, NJ) and cytokines such as □-interferon, IL-2, or IL-12 or genes encoding proteins involved in immune helper functions, such as B-7.

### B. Lipid Components & Moieties

In some aspects, the composition further includes a lipid-based delivery system (e.g., a lipid-based vaccine), which delivers a nucleic acid molecule to the interior of a cell, where it can then replicate, inhibit protein expression of interest, and/or express the encoded polypeptide of interest. The delivery system may have adjuvant effects which enhance the immunogenicity of an encoded antigen.

Thus, in certain aspects, the present invention concerns compositions comprising one or more lipids associated with a nucleic acid or a polypeptide/peptide. A lipid is a substance that is insoluble in water and extractable with an organic solvent. Compounds other than those specifically described herein are understood by one of skill in the art as lipids, and are encompassed by the compositions and methods of the present invention. A lipid component and a non-lipid may be attached to one another, either covalently or non-covalently.

A lipid may be a naturally occurring lipid or a synthetic lipid. However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof.

In some aspects, the composition further includes neutral lipids, cationic lipids, cholesterol, and polyethylene glycol (PEG), and forms nanoparticles that encompass, or encapsulate, the nucleic acid molecules. In some aspects, the composition further includes any one of a cationic lipid, a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, and a cationic nanoemulsion. In some aspects, the nucleic acid molecule is encapsulated in, bound to or adsorbed on any one of a cationic lipid, a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, and a cationic nanoemulsion, or a combination thereof. As used herein, “encapsulate,” “encapsulated,” “encapsulation,” and grammatically comparable variants thereof mean that at least a portion of a substance is enclosed or surrounded by another material or another substance in a composition. In some aspects, a substance, such as a nucleic acid, can be fully enclosed or surrounded by another material or another substance in a composition, such as a lipid.

In certain aspects, a composition may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or any range therebetween, of a particular lipid, lipid type, or non-lipid component such as an adjuvant, antigen, peptide, polypeptide, sugar, nucleic acid or other material disclosed herein or as would be known to one of skill in the art.

### C. General Pharmaceutical Compositions

In some aspects, the compositions further comprise one or more stabilizing agents and one or more buffers. A nucleic acid molecule, e.g., a naked or encapsulated nucleic acid, or a polypeptide as disclosed herein may be comprised in a solution comprising the one or more stabilizing agents and one or more buffers. In some aspects, the stabilizing agent comprises sucrose, mannose, sorbitol, raffinose, trehalose, mannitol, inositol, sodium chloride, arginine, lactose, hydroxyethyl starch, dextran, polyvinylpyrolidone, glycine, or a combination thereof. In some aspects, the stabilizing agent is a disaccharide, or sugar. In one aspect, the stabilizing agent is sucrose. In another aspect, the stabilizing agent is trehalose. In a further aspect, the stabilizing agent is a combination of sucrose and trehalose. In some aspects, the total concentration of the stabilizing agent(s) in the composition is about 50% to about 10% w/v. For example, the total concentration of the stabilizing agent can be equal to any one of, at least any one of, at most any one of, or between any two of 1%, 2%, 3%, 4%, 5%, 6%, 7%, 8%, 9%, 10%, 11%, 12%, 13%, 14%, 15%, 16%, 17%, 18%, 19%, or 20% w/v or any range or value derivable therein. In specific aspects, the total concentration of the stabilizing agent(s) in the composition is 10% w/v. In specific aspects, the amino acid concentration is 5% w/v.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. In some aspects, the buffer is a HEPES buffer, a Tris buffer, or a PBS buffer. In one aspect, the buffer is Tris buffer. In another aspect, the buffer is a HEPES buffer. In a further aspect, the buffer is a PBS buffer. In some aspects, the concentration of the buffer in the composition is about 10 mM. For example, the buffer concentration can be equal to any one of, at least any one of, at most any one of, or between any two of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM, or any range or value derivable therein. In specific aspects, the buffer concentration is 10 mM. The buffer can be at a neutral pH, pH 6.5 to 8.5, pH 7.0 to pH 8.0, or pH 7.2 to pH 7.6. For example, the buffer can be at pH 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5, or any range or value derivable therein. In specific aspects, the buffer is at pH 7.4.

The compositions may further include one or more salts and/or one or more pharmaceutically acceptable surfactants, preservatives, carriers, diluents, and/or excipients, in some cases. In some aspects, the composition further includes a pharmaceutically acceptable vehicle. In some aspects, each of a buffer, stabilizing agent, salt, surfactant, preservative, and excipient are included in the compositions. In other aspects, any one or more of a buffer, stabilizing agent, salt, surfactant, preservative, excipient, carrier, diluent, or vehicle may be excluded from compositions.

Examples of salts include but not limited to sodium salts and/or potassium salts. In some aspects, the sodium salt comprises sodium chloride. In some aspects, the potassium salt comprises potassium chloride. The concentration of the salts in the composition can be about 70 mM to about 140 mM. For example, the salt concentration can be equal to any one of, at least any one of, at most any one of, or between any two of 50 mM, 60 mM, 70 mM, 80 mM, 90 mM, 100 mM, 120 mM, 130 mM, 140 mM, 150 mM, 160 mM, 170 mM, 180 mM, 190 mM, or 200 mM, or any range or value derivable therein. In specific aspects, the salt concentration is 70 mM. In specific aspects, the salt concentration is 140 mM. The salt can be at a neutral pH, pH 6.5 to 8.5, pH 7.0 to pH 8.0, or pH 7.2 to pH 7.6. For example, the salt can be at a pH equal to any one of, at least any one of, at most any one of, or between any two of 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5, or any range or value derivable therein.

Examples of excipients, which refer to ingredients in the compositions that are not active ingredients, include but are not limited to carriers, binders, diluents, lubricants, thickeners, surface active agents, preservatives, stabilizers, emulsifiers, buffers, flavoring agents, disintegrants, coatings, plasticizers, compression agents, wet granulation agents, or colorants. Preservatives for use in the compositions disclosed herein include but are not limited to benzalkonium chloride, chlorobutanol, paraben and thimerosal. As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. Diluents, or diluting or thinning agents, include but are not limited to ethanol, glycerol, water, sugars such as lactose, sucrose, mannitol, and sorbitol, and starches derived from wheat, corn rice, and potato; and celluloses such as microcrystalline cellulose. The amount of diluent in the composition can range from about 10% to about 90% by weight of the total composition, about 25% to about 75%, about 30% to about 60% by weight, or about 12% to about 60%.

The pH and exact concentration of the various components in a pharmaceutical composition are adjusted according to well-known parameters. The use of such media and agents for pharmaceutical active substances is well known in the art. Except insofar as any conventional media or agent is incompatible with the active ingredients, its use in immunogenic, prophylactic and/or therapeutic compositions is contemplated.

### D. Combination Therapy

The compositions and related methods of the present invention, particularly administration of a coronavirus protein, including an isolated polypeptide comprising a native or variant coronavirus spike protein or fragment thereof, may also be used in combination with the administration of traditional therapies. These include, but are not limited to, the administration of antiviral therapies such as nirmatrelvir/ritonavir, remdesivir, or various combinations of antivirals. Also included are the administration of steroids including corticosteroids, e.g., dexamethasone, anti-inflammatories including acetaminophen or ibuprofen, or various combinations thereof.

In one aspect, it is contemplated that a vaccine and/or therapy is used in conjunction with antiviral treatment. Alternatively, the therapy may precede or follow the other agent treatment by intervals ranging from minutes to weeks. In aspects where the other agents and/or vaccines are administered separately, one would generally ensure that a significant period of time did not expire between the time of each delivery, such that the agent and immunogenic composition would still be able to exert an advantageously combined effect on the subject. In such instances, it is contemplated that one may administer both modalities within about 12-24 h of each other or within about 6-12 h of each other. In some situations, it may be desirable to extend the time period for administration significantly, where several days (2, 3, 4, 5, 6 or 7) to several weeks (1, 2, 3, 4, 5, 6, 7 or 8) lapse between the respective administrations.

Various combinations may be employed, for example antiviral therapy is “A” and the immunogenic polypeptide given as part of an immune therapy regime is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the immunogenic compositions of the present invention to a patient/subject will follow general protocols for the administration of such compounds, taking into account the toxicity, if any, of the coronavirus spike protein composition, or other compositions described herein. It is expected that the treatment cycles would be repeated as necessary. It also is contemplated that various standard therapies, such as hydration, may be applied in combination with the described therapy.

### E. Administration

Administration of the compositions described herein can be carried out via any of the accepted modes of administration of agents for serving similar utilities. Pharmaceutical compositions may be formulated into preparations in solid, semi-solid, liquid or gaseous forms, such as tablets, capsules, powders, granules, ointments, solutions, suspensions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection, or infusion techniques. Pharmaceutical compositions described herein are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound in aerosol form may hold a plurality of dosage units. The composition to be administered will, in any event, contain a therapeutically and/or prophylactically effective amount of a compound within the scope of this disclosure, or a pharmaceutically acceptable salt thereof, for treatment of a disease or condition of interest in accordance with the teachings described herein.

A pharmaceutical composition within the scope of this disclosure may be in the form of a solid or liquid. In one aspect, the carrier(s) are particulate, so that the compositions are, for example, in tablet or powder form. The carrier(s) may be liquid, with the compositions being, for example, an oral syrup, injectable liquid, or an aerosol, which is useful in, for example, inhalator administration. When intended for oral administration, the pharmaceutical composition is preferably in either solid or liquid form, where semi-solid, semi-liquid, suspension, and gel forms are included within the forms considered herein as either solid or liquid. As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present or exclude: binders such as carboxymethylcellulose, ethyl cellulose, microcrystalline cellulose, gum tragacanth, or gelatin; excipients such as starch, lactose, or dextrins; disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate, or orange flavoring; and a coloring agent. When the pharmaceutical composition is in the form of a capsule, for example, a gelatin capsule, it may contain, in addition to materials of the above type, a liquid carrier such as polyethylene glycol or oil. The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant, and flavor enhancer. In a composition intended to be administered by injection, one or more of a surfactant, preservative, wetting agent, dispersing agent, suspending agent, buffer, stabilizer, and isotonic agent may be included or exclude.

A liquid pharmaceutical composition, whether they be solutions, suspensions, or other like form, may include or exclude one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates, or phosphates; and agents for the adjustment of tonicity such as sodium chloride or dextrose; agents to act as cryoprotectants such as sucrose or trehalose. The parenteral preparation can be enclosed in ampoules, disposable syringes, or multiple dose vials made of glass or plastic. Physiological saline is a preferred adjuvant. An injectable pharmaceutical composition is preferably sterile.

A liquid pharmaceutical composition intended for either parenteral or oral administration should contain an amount of a compound such that a suitable dosage will be obtained.

The pharmaceutical compositions may be prepared by methodology well known in the pharmaceutical art. For example, a pharmaceutical composition intended to be administered by injection can be prepared by combining the nucleic acid or polypeptide with sterile, distilled water or other carrier so as to form a solution. A surfactant may be added to facilitate the formation of a homogeneous solution or suspension. Surfactants are compounds that non-covalently interact with a compound consistent with the teachings herein so as to facilitate dissolution or homogeneous suspension of the compound in the aqueous delivery system.

The compositions within the scope of the disclosure are administered in a therapeutically and/or prophylactically effective amount, which will vary depending upon a variety of factors including the activity of the specific therapeutic and/or prophylactic agent employed; the metabolic stability and length of action of the therapeutic and/or prophylactic agent; the age, body weight, general health, gender, and diet of the patient; the mode and time of administration; the rate of excretion; the drug combination; the severity of the particular disorder or condition; and the subject undergoing therapy.

### IX. Methods of Variant Protein Selection and Use

Methods of the present disclosure include treatment for a disease or condition caused by a coronavirus. An immunogenic polypeptide of the disclosure can be given to induce an immune response in a person infected with a coronavirus or suspected of having been exposed to a coronavirus. An immunogenic polypeptide of the disclosure can be given to induce an immune response in a person at risk of being infected with a coronavirus or suspected of being at risk of exposure to a coronavirus. Methods may be employed with respect to individuals who have tested positive for exposure to coronavirus or who are deemed to be at risk for infection based on possible exposure.

In particular, the disclosure encompasses a method of treating, preventing, or reducing the severity of infection caused by coronavirus. In some aspects, the treatment is administered in the absence of adjuvants or carriers or other coronavirus antigens. In some aspects, the treatment is administered in the presence of adjuvants or carriers or other coronavirus antigens. Furthermore, in some examples, treatment comprises administration of other agents commonly used against viral infection, such as one or more antivirals.

In one aspect, the disclosure relates to a method for producing an immunogenic composition (e.g., a vaccine) comprising an immunogenic polypeptide construct and/or a nucleic acid encoding the immunogenic polypeptide construct (e.g., a coronavirus spike protein or fragment thereof). The method can comprise one or both of the following steps: (1) identifying at least one modification to be made to a native, unmodified coronavirus spike protein that would (a) increase adoption by RBDs of the variant coronavirus spike proteins of the RBD-up conformation to expose more neutralization-sensitive epitopes on the spike protein, (b) decrease adoption by RBDs of the variant coronavirus spike proteins of the RBD-down conformation, (c) increase expression of the variant coronavirus spike protein compared to the native coronavirus spike protein, (d) increase adoption of a prefusion conformation, (e) decrease shedding of a S1 subunit of the variant coronavirus spike protein, and/or (f) improve localization of the variant coronavirus spike protein to a host cell membrane; and (2) modifying the antigenic peptide sequence of the native, unmodified coronavirus spike protein according to the identified modifications to form a variant coronavirus spike protein construct and/or a nucleic acid encoding the variant coronavirus spike protein construct.

In some aspects, the method comprises both of the steps of (1) identifying at least one modification to be made to a native, unmodified coronavirus spike protein that would (a) increase adoption by RBDs of the variant coronavirus spike proteins of the RBD-up conformation to expose more neutralization-sensitive epitopes on the spike protein, (b) decrease adoption by RBDs of the variant coronavirus spike proteins of the RBD-down conformation, (c) increase expression of the variant coronavirus spike protein compared to the native coronavirus spike protein, (d) increase adoption of a prefusion conformation, (e) decrease shedding of a S1 subunit of the variant coronavirus spike protein, and/or (f) improve localization of the variant coronavirus spike protein to a host cell membrane; and (2) modifying the antigenic peptide sequence of the native, unmodified coronavirus spike protein according to the identified modifications to form a variant coronavirus spike protein construct and/or a nucleic acid encoding the variant coronavirus spike protein construct.

In some aspects, modifications to native coronavirus spike proteins to produce variant coronavirus spike proteins that would (a) increase adoption by RBDs of the variant coronavirus spike proteins of the RBD-up conformation to expose more neutralization-sensitive epitopes on the spike protein, (b) decrease adoption by RBDs of the variant coronavirus spike proteins of the RBD-down conformation, (c) increase expression of the variant coronavirus spike protein compared to the native coronavirus spike protein, (d) increase adoption of a prefusion conformation, (e) decrease shedding of a S1 subunit of the variant coronavirus spike protein, and/or (f) improve localization of the variant coronavirus spike protein to a host cell membrane can be identified using deep learning methods that utilize the coronavirus genomic sequences compiled by the GISAID database (found on the World Wide Web at GSAID.org). The deep learning methods can analyze the genotype-phenotype relationship between the genomic sequences; perform deep mutational scans of the RBDs of the sequences, including analysis for ACE2 binding and/or antibody escape mutations, to assess antigenic drift; and/or perform structural modeling of the variant spike proteins. In some aspects, deep learning methods can be used to model and predict variant coronavirus strains such that isolated immunogenic polypeptides comprising the spike proteins of these identified variants can be generated for use in treating, preventing, or reducing the severity of infection caused by coronavirus as described herein.

In some aspects, a population of isolated immunogenic polypeptides comprising variant coronavirus spike proteins that are variants of a native coronavirus spike protein or fragments thereof of the present disclosure comprises a plurality of variant coronavirus spike proteins in the RBD-up conformation. In some aspects, the population of the variant coronavirus spike proteins in the RBD-up conformation is greater compared to a population of native coronavirus spike proteins and/or alternative variant coronavirus spike proteins. In some aspects, the population of the variant coronavirus spike proteins of the present disclosure in the RBD-up conformation is greater compared to a population of native coronavirus spike proteins and/or alternative variant coronavirus spike proteins by equal to any one of, at least any one of, at most any one of, or between any two of about 5% or higher, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 1-fold or more, about 2-fold or more, about 3-fold or more, about 4-fold or more, about 5-fold or more, about 10-fold or more, about 20-fold or more, about 30-fold or more, about 40-fold or more, about 50-fold or more, about 100-fold or more, about 200-fold or more, about 300-fold or more, about 400-fold or more, about 500-fold or more, about 1000-fold or more, about 2000-fold or more, about 3000-fold or more, about 4000-fold or more, about 5000-fold or more, or about 10000-fold or more.

In some aspects, the population of the variant coronavirus spike proteins in the RBD-down conformation is decreased compared to a population of native coronavirus spike proteins and/or alternative variant coronavirus spike proteins. In some aspects, the population of the variant coronavirus spike proteins of the present disclosure in the RBD-down conformation is decreased compared to a population of native coronavirus spike proteins and/or alternative variant coronavirus spike proteins by equal to any one of, at least any one of, at most any one of, or between any two of about 5% or higher, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 1-fold or more, about 2-fold or more, about 3-fold or more, about 4-fold or more, about 5-fold or more, about 10-fold or more, about 20-fold or more, about 30-fold or more, about 40-fold or more, about 50-fold or more, about 100-fold or more, about 200-fold or more, about 300-fold or more, about 400-fold or more, about 500-fold or more, about 1000-fold or more, about 2000-fold or more, about 3000-fold or more, about 4000-fold or more, about 5000-fold or more, or about 10000-fold or more.

In some aspects, expression is increased of a population of isolated immunogenic polypeptides comprising variant coronavirus spike proteins that are variants of a native coronavirus spike protein or fragments thereof of the present disclosure. In some aspects, expression of the population of the variant coronavirus spike proteins of the present disclosure is increased compared to expression of a population of native coronavirus spike proteins and/or alternative variant coronavirus spike proteins. In some aspects, expression of the population of the variant coronavirus spike proteins of the present disclosure is greater compared to a population of native coronavirus spike proteins and/or alternative variant coronavirus spike proteins by equal to any one of, at least any one of, at most any one of, or between any two of about 5% or higher, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 1-fold or more, about 2-fold or more, about 3-fold or more, about 4-fold or more, about 5-fold or more, about 10-fold or more, about 20-fold or more, about 30-fold or more, about 40-fold or more, about 50-fold or more, about 100-fold or more, about 200-fold or more, about 300-fold or more, about 400-fold or more, about 500-fold or more, about 1000-fold or more, about 2000-fold or more, about 3000-fold or more, about 4000-fold or more, about 5000-fold or more, or about 10000-fold or more.

In some aspects, a population of isolated immunogenic polypeptides comprising variant coronavirus spike proteins that are variants of a native coronavirus spike protein or fragments thereof of the present disclosure is stabilized in a prefusion conformation. In some aspects, the population of the variant coronavirus spike proteins in a stabilized prefusion conformation is greater compared to a population of native coronavirus spike proteins and/or alternative variant coronavirus spike proteins. In some aspects, the population of the variant coronavirus spike proteins in a stabilized prefusion conformation is greater compared to a population of native coronavirus spike proteins and/or alternative variant coronavirus spike proteins by equal to any one of, at least any one of, at most any one of, or between any two of about 5% or higher, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 1-fold or more, about 2-fold or more, about 3-fold or more, about 4-fold or more, about 5-fold or more, about 10-fold or more, about 20-fold or more, about 30-fold or more, about 40-fold or more, about 50-fold or more, about 100-fold or more, about 200-fold or more, about 300-fold or more, about 400-fold or more, about 500-fold or more, about 1000-fold or more, about 2000-fold or more, about 3000-fold or more, about 4000-fold or more, about 5000-fold or more, or about 10000-fold or more.

In some aspects, shedding of a S1 subunit by a population of isolated immunogenic polypeptides comprising variant coronavirus spike proteins that are variants of a native coronavirus spike protein or fragments thereof of the present disclosure is decreased. In some aspects, shedding of a S1 subunit by a population of the variant coronavirus spike proteins of the present disclosure is decreased compared to a population of native coronavirus spike proteins and/or alternative variant coronavirus spike proteins. In some aspects, shedding of a S1 subunit by a population of the variant coronavirus spike proteins of the present disclosure is decreased compared to a population of native coronavirus spike proteins and/or alternative variant coronavirus spike proteins by equal to any one of, at least any one of, at most any one of, or between any two of about 5% or higher, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 1-fold or more, about 2-fold or more, about 3-fold or more, about 4-fold or more, about 5-fold or more, about 10-fold or more, about 20-fold or more, about 30-fold or more, about 40-fold or more, about 50-fold or more, about 100-fold or more, about 200-fold or more, about 300-fold or more, about 400-fold or more, about 500-fold or more, about 1000-fold or more, about 2000-fold or more, about 3000-fold or more, about 4000-fold or more, about 5000-fold or more, or about 10000-fold or more.

In some aspects, localization of a population of isolated immunogenic polypeptides comprising variant coronavirus spike proteins that are variants of a native coronavirus spike protein or fragments thereof of the present disclosure to a host cell membrane is improved. In some aspects, localization of a population of the variant coronavirus spike proteins of the present disclosure to a host cell membrane is improved compared to a population of native coronavirus spike proteins and/or alternative variant coronavirus spike proteins. In some aspects, localization of a population of the variant coronavirus spike proteins of the present disclosure to a host cell membrane is improved compared to a population of native coronavirus spike proteins and/or alternative variant coronavirus spike proteins by equal to any one of, at least any one of, at most any one of, or between any two of about 5% or higher, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 1-fold or more, about 2-fold or more, about 3-fold or more, about 4-fold or more, about 5-fold or more, about 10-fold or more, about 20-fold or more, about 30-fold or more, about 40-fold or more, about 50-fold or more, about 100-fold or more, about 200-fold or more, about 300-fold or more, about 400-fold or more, about 500-fold or more, about 1000-fold or more, about 2000-fold or more, about 3000-fold or more, about 4000-fold or more, about 5000-fold or more, or about 10000-fold or more.

In some aspects, the isolated immunogenic polypeptides comprising variant coronavirus spike proteins that are variants of a native coronavirus spike protein or fragments thereof of the present disclosure and/or compositions thereof produced by the methods of the present disclosure have an efficacy, intracellular delivery, and/or immunogenicity equivalent to or higher than the efficacy, intracellular delivery, and/or immunogenicity of a native, unmodified coronavirus spike protein and/or a native, unmodified coronavirus spike protein construct and/or compositions thereof.

In some aspects, a population of isolated immunogenic polypeptides comprising variant coronavirus spike proteins that are variants of a native coronavirus spike protein or fragments thereof of the present disclosure have an efficacy, intracellular delivery, and/or immunogenicity higher than the efficacy, intracellular delivery, and/or immunogenicity of a native, unmodified coronavirus spike protein and/or a native, unmodified coronavirus spike protein construct and/or compositions thereof by equal to any one of, at least any one of, at most any one of, or between any two of about 5% or higher, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 1-fold or more, about 2-fold or more, about 3-fold or more, about 4-fold or more, about 5-fold or more, about 10-fold or more, about 20-fold or more, about 30-fold or more, about 40-fold or more, about 50-fold or more, about 100-fold or more, about 200-fold or more, about 300-fold or more, about 400-fold or more, about 500-fold or more, about 1000-fold or more, about 2000-fold or more, about 3000-fold or more, about 4000-fold or more, about 5000-fold or more, or about 10000-fold or more.

In one aspect, the disclosure relates to a method for producing an immunogenic composition comprising an immunogenic polypeptide construct and/or a nucleic acid encoding the immunogenic polypeptide construct. The method can comprise one or both of the following steps: (1) identifying at least one modification to be made to a native, unmodified coronavirus spike protein that would (a) increase adoption by RBDs of the variant coronavirus spike proteins of the RBD-up conformation to expose more neutralization-sensitive epitopes on the spike protein, (b) decrease adoption by RBDs of the variant coronavirus spike proteins of the RBD-down conformation, (c) increase expression of the variant coronavirus spike protein compared to the native coronavirus spike protein, (d) increase adoption of a prefusion conformation, (e) decrease shedding of a S1 subunit of the variant coronavirus spike protein, and/or (f) improve localization of the variant coronavirus spike protein to a host cell membrane; and (2) modifying the antigenic peptide sequence of the native, unmodified coronavirus spike protein according to the identified modifications to form a variant coronavirus spike protein construct and/or a nucleic acid encoding the variant coronavirus spike protein construct.

In another aspect, the disclosure relates to a method of inducing an immune response in a subject. The method includes administering to the mammalian cell an effective amount of a composition as described herein.

In another aspect, the disclosure relates to a method of vaccinating a subject. The method includes administering to the subject in need thereof an effective amount of a composition described herein.

In another aspect, the disclosure relates to a method of treating or preventing or reducing the severity of an infectious disease. The method includes administering to the subject an effective amount of a composition as described herein.

In another aspect, disclosure relates to a method of treating or preventing or reducing the severity of an infectious disease in a subject by, for example, inducing an immune response to an infectious disease in the subject. In some aspects, the method includes administering a priming composition that includes an effective amount of a composition described herein, and administering a booster composition including an effective amount of an adenoviral vector encoding an antigen. In another aspect, the method includes administering a priming composition including an effective amount of an adenoviral vector encoding an antigen, and administering a booster composition that includes an effective amount of a composition described herein. In some aspects, the composition elicits an immune response including an antibody response. In some aspects, the composition elicits an immune response including a T cell response.

In another aspect, the disclosure relates to a method of treating or preventing or reducing the severity of a coronavirus infection and/or illness caused by the coronavirus. The method includes administering to the subject an effective amount of a composition as described herein.

In another aspect, disclosure relates to a method of treating or preventing or reducing the severity of a coronavirus infection and/or illness caused by the coronavirus in a subject by, for example, inducing an immune response to a coronavirus infection in the subject. In some aspects, the method includes administering a priming composition that includes an effective amount of a composition described herein, and administering a booster composition including an effective amount of an adenoviral vector encoding an antigen. In another aspect, the method includes administering a priming composition including an effective amount of an adenoviral vector encoding an antigen, and administering a booster composition that includes an effective amount of a composition described herein. In some aspects, the composition elicits an immune response including an antibody response. In some aspects, the composition elicits an immune response including a T cell response.

In another aspect, the disclosure relates to use of an isolated immunogenic polypeptide as described herein in the manufacture of an immunogenic composition for treatment or prevention of an infectious disease. In another aspect, the disclosure relates to use of an isolated immunogenic polypeptide as described herein in the manufacture of an immunogenic composition for treatment or prevention of a coronavirus infection.

### EXAMPLES

The following examples are included to demonstrate aspects of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the disclosure.

### Example 1

*The amino acid positions in Example 1, Table 6, and the figures associated therewith are amino acid positions from a SARS-CoV-2 spike protein sequence as found in the UniProt Accession Number P0DTC2-1 sequence.

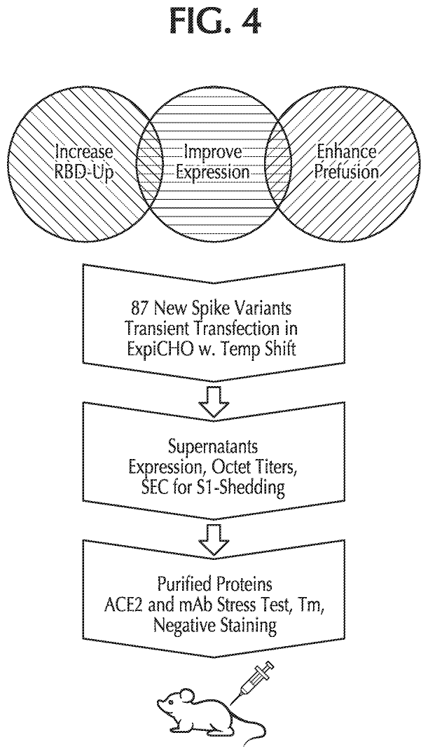

A strategy employed for identifying, producing, and testing variant coronavirus spike protein constructs is illustrated in FIG. 4. In particular, in some embodiments, variants are selected based at least in part on their ability to increase the RBD-up conformation of the spike protein, improve expression of the spike protein, and/or increase adoption of a prefusion conformation. Cells are transiently transfected with expression constructs encoding the selected variants, the variants are expressed by the cells, and the expressed variants are purified. Supernatants collected during purification of the variants are analyzed to determine variant expression, octet titers, and amount of S1 subunit shedding. ACE2 and mAb stress testing, Tm, and negative staining is performed to further characterize the variants. Finally, in vivo testing with the variants is conducted to assess immunogenicity and tolerability of the variants.

The 87 variants tested and referenced in FIG. 4 are shown below in Table 6. In Table 6, the mutant variant numbering is under the Column “Code.” Each mutant variant includes the mutations listed on the respective row. As an example, mutant 66 is represented on the Code row for “066,” which has the following mutations: D614G, F817P, A892P, A899P, A942P, V987P, I980C-Q992C, and RRAR (682-685)→GSAS, according to the amino acid positions from a SARS-CoV-2 spike protein sequence as found in the UNiProt Accession Number P0DTC2-1 sequence. As another example, mutant 70 is represented on the row for “070,” which has the following mutations: D614G, F817P, A892P, A899P, A942P, D985P, V987P, RRAR (682-685)→GSAS, and F329S, according to the amino acid positions from a SARS-CoV-2 spike protein sequence as found in the UniProt Accession Number P0TC2-1 sequence.

TABLE 6
Mutations

| | | | Block pre-to- | | RBD head |
|---|---|---|---|---|---|
| Code | D614G | Proline Mutations | postfusion | Reduce Shedding | up/down |
| 01 | D614G | P2 (K986P, V987P) | n/a | RRAR (682-685) → GSAS | n/a |
| 02 | D614G | P6 (F817P, A892P, A899P, | n/a | RRAR (682-685) → GSAS | n/a |
| | | A942P, K986P, V987P) | | | |
| 03 | D614G | P6 (F817P, A892P, A899P, | n/a | RRAR (682-685) → GSAS | S383C-D985C |
| | | A942P, K986P, V987P) | | | |
| 04 | WT | P2 (K986P, V987P) | n/a | RRAR (682-685) → GSAS | n/a |
| 05 | WT | P6 (F817P, A892P, A899P, | n/a | RRAR (682-685) → GSAS | n/a |
| | | A942P, K986P, V987P) | | | |
| 06 | D614G | F817P, A892P, A899P, | n/a | RRAR (682-685) → GSAS | n/a |
| | | A942P, V987P | | | |
| 07 | D614G | F817P, A892P, A899P, | n/a | RRAR (682-685) → GSAS | n/a |
| | | A942P, D985P, V987P | | | |
| 08 | D614G | F817P, A892P, A899P, | n/a | RRAR (682-685) → GSAS | n/a |
| | | A942P, D985P, K986P, | | | |
| | | V987P | | | |
| 09 | D614G | F817P, A892P, A899P, | n/a | RRAR (682-685) → GSAS | n/a |
| | | A942P, D985P, K986P | | | |
| 10 | D614G | F817P, A892P, A899P, | n/a | RRAR (682-685) → GSAS | n/a |
| | | S943P, K986P, V987P | | | |
| 11 | D614G | F817P, A892P, A899P, | n/a | RRAR (682-685) → GSAS | n/a |
| | | A944P, K986P, V987P | | | |
| 12 | D614G | P6 (F817P, A892P, A899P, | n/a | RRAR (682-685) → GSAS | V367F |
| | | A942P, K986P, V987P) | | | |
| 13 | D614G | P6 (F817P, A892P, A899P, | n/a | RRAR (682-685) → GSAS | F329Y |
| | | A942P, K986P, V987P) | | | |
| 14 | D614G | P6 (F817P, A892P, A899P, | n/a | RRAR (682-685) → GSAS | F329S |
| | | A942P, K986P, V987P) | | | |
| 15 | D614G | P6 (F817P, A892P, A899P, | n/a | RRAR (682-685) → GSAS | D568N |
| | | A942P, K986P, V987P) | | | |
| 16 | D614G | P6 (F817P, A892P, A899P, | n/a | RRAR (682-685) → GSAS | E619Q |
| | | A942P, K986P, V987P) | | | |
| 17 | D614G | P6 (F817P, A892P, A899P, | n/a | RRAR (682-685) → GSAS | D830N |
| | | A942P, K986P, V987P) | | | |
| 18 | D614G | P6 (F817P, A892P, A899P, | n/a | RRAR (682-685) → GSAS | D839N |
| | | A942P, K986P, V987P) | | | |
| 19 | D614G | P6 (F817P, A892P, A899P, | n/a | RRAR (682-685) → GSAS | D843N |
| | | A942P, K986P, V987P) | | | |
| 20 | D614G | P6 (F817P, A892P, A899P, | n/a | RRAR (682-685) → GSAS | D848N |
| | | A942P, K986P, V987P) | | | |
| 21 | D614G | P6 (F817P, A892P, A899P, | n/a | RRAR (682-685) → GSAS | T827C-Q949C |
| | | A942P, K986P, V987P) | | | |
| 22 | D614G | P6 (F817P, A892P, A899P, | A944P | RRAR (682-685) → GSAS | n/a |
| | | A942P, K986P, V987P) | | | |
| 23 | D614G | P6 (F817P, A892P, A899P, | S943P | RRAR (682-685) → GSAS | n/a |
| | | A942P, K986P, V987P) | | | |
| 24 | D614G | P6 (F817P, A892P, A899P, | N919Y | RRAR (682-685) → GSAS | n/a |
| | | A942P, K986P, V987P) | | | |
| 25 | D614G | P6 (F817P, A892P, A899P, | L981Y | RRAR (682-685) → GSAS | n/a |
| | | A942P, K986P, V987P) | | | |
| 26 | D614G | P6 (F817P, A892P, A899P, | V1061I | RRAR (682-685) → GSAS | n/a |
| | | A942P, K986P, V987P) | | | |
| 27 | D614G | P6 (F817P, A892P, A899P, | S975P | RRAR (682-685) → GSAS | n/a |
| | | A942P, K986P, V987P) | | | |
| 28 | D614G | P6 (F817P, A892P, A899P, | N969P | RRAR (682-685) → GSAS | n/a |
| | | A942P, K986P, V987P) | | | |
| 29 | D614G | P6 (F817P, A892P, A899P, | N955L | RRAR (682-685) → GSAS | n/a |
| | | A942P, K986P, V987P) | | | |
| 30 | D614G | P6 (F817P, A892P, A899P, | L938F | RRAR (682-685) → GSAS | n/a |
| | | A942P, K986P, V987P) | | | |
| 31 | D614G | P6 (F817P, A892P, A899P, | N856L | RRAR (682-685) → GSAS | n/a |
| | | A942P, K986P, V987P) | | | |
| 32 | D614G | P6 (F817P, A892P, A899P, | R815M | RRAR (682-685) → GSAS | n/a |
| | | A942P, K986P, V987P) | | | |
| 33 | D614G | P6 (F817P, A892P, A899P, | A845P | RRAR (682-685) → GSAS | n/a |
| | | A942P, K986P, V987P) | | | |
| 34 | D614G | P6 (F817P, A892P, A899P, | S746I | RRAR (682-685) → GSAS | n/a |
| | | A942P, K986P, V987P) | | | |
| 35 | D614G | P6 (F817P, A892P, A899P, | T734I, Y1007F | RRAR (682-685) → GSAS | n/a |
| | | A942P, K986P, V987P) | | | |
| 36 | D614G | P6 (F817P, A892P, A899P, | L948C-G1059C | RRAR (682-685) → GSAS | n/a |
| | | A942P, K986P, V987P) | | | |
| 37 | D614G | P6 (F817P, A892P, A899P, | F970C-G999C | RRAR (682-685) → GSAS | n/a |
| | | A942P, K986P, V987P) | | | |
| 38 | D614G | P6 (F817P, A892P, A899P, | A972C-Q992C | RRAR (682-685) → GSAS | n/a |
| | | A942P, K986P, V987P) | | | |
| 39 | D614G | P6 (F817P, A892P, A899P, | I980C-Q992C | RRAR (682-685) → GSAS | n/a |
| | | A942P, K986P, V987P) | | | |
| 40 | D614G | P6 (F817P, A892P, A899P, | S967C-S975C | RRAR (682-685) → GSAS | n/a |
| | | A942P, K986P, V987P) | | | |
| 41 | D614G | P6 (F817P, A892P, A899P, | L984C-A989C | RRAR (682-685) → GSAS | n/a |
| | | A942P, K986P, V987P) | | | |
| 42 | D614G | P6 (F817P, A892P, A899P, | L984C-E988C | RRAR (682-685) → GSAS | n/a |
| | | A942P, K986P, V987P) | | | |
| 43 | D614G | P6 (F817P, A892P, A899P, | Q755C-N969C | RRAR (682-685) → GSAS | n/a |
| | | A942P, K986P, V987P) | | | |
| 44 | D614G | P6 (F817P, A892P, A899P, | T791C-A879C | RRAR (682-685) → GSAS | n/a |
| | | A942P, K986P, V987P) | | | |
| 45 | D614G | P6 (F817P, A892P, A899P, | S884C-A893C | RRAR (682-685) → GSAS | n/a |
| | | A942P, K986P, V987P) | | | |
| 46 | D614G | P6 (F817P, A892P, A899P, | n/a | K854F | n/a |
| | | A942P, K986P, V987P) | | | |
| 47 | D614G | P6 (F817P, A892P, A899P, | n/a | A609C-I692C | n/a |
| | | A942P, K986P, V987P) | | | |
| 48 | D614G | P6 (F817P, A892P, A899P, | n/a | K304E | n/a |
| | | A942P, K986P, V987P) | | | |
| 49 | D614G | P6 (F817P, A892P, A899P, | n/a | D571C-S967C | n/a |
| | | A942P, K986P, V987P) | | | |
| 50 | D614G | P6 (F817P, A892P, A899P, | n/a | A570C-N960C | n/a |
| | | A942P, K986P, V987P) | | | |
| 51 | D614G | F817P, A892P, A899P, | n/a | RRAR (682-685) → GSAS | V367F |
| | | A942P, V987P | | | |
| 52 | D614G | F817P, A892P, A899P, | n/a | RRAR (682-685) → GSAS | F329S |
| | | A942P, V987P | | | |
| 53 | D614G | F817P, A892P, A899P, | n/a | RRAR (682-685) → GSAS | D568N |
| | | A942P, V987P | | | |
| 54 | D614G | F817P, A892P, A899P, | n/a | RRAR (682-685) → GSAS | E619Q |
| | | A942P, V987P | | | |
| 55 | D614G | F817P, A892P, A899P, | n/a | RRAR (682-685) → GSAS | D830N |
| | | A942P, V987P | | | |
| 56 | D614G | F817P, A892P, A899P, | n/a | RRAR (682-685) → GSAS | D839N |
| | | A942P, V987P | | | |
| 57 | D614G | F817P, A892P, A899P, | n/a | RRAR (682-685) → GSAS | D843N |
| | | A942P, V987P | | | |
| 58 | D614G | F817P, A892P, A899P, | n/a | RRAR (682-685) → GSAS | D848N |
| | | A942P, V987P | | | |
| 59 | D614G | F817P, A892P, A899P, | A944P | RRAR (682-685) → GSAS | n/a |
| | | A942P, V987P | | | |
| 60 | D614G | F817P, A892P, A899P, | S943P | RRAR (682-685) → GSAS | n/a |
| | | A942P, V987P | | | |
| 61 | D614G | F817P, A892P, A899P, | N919Y | RRAR (682-685) → GSAS | n/a |
| | | A942P, V987P | | | |
| 62 | D614G | F817P, A892P, A899P, | N856L | RRAR (682-685) → GSAS | n/a |
| | | A942P, V987P | | | |
| 63 | D614G | F817P, A892P, A899P, | L948C-G1059C | RRAR (682-685) → GSAS | n/a |
| | | A942P, V987P | | | |
| 64 | D614G | F817P, A892P, A899P, | F970C-G999C | RRAR (682-685) → GSAS | n/a |
| | | A942P, V987P | | | |
| 65 | D614G | F817P, A892P, A899P, | A972C-Q992C | RRAR (682-685) → GSAS | n/a |
| | | A942P, V987P | | | |
| 66 | D614G | F817P, A892P, A899P, | I980C-Q992C | RRAR (682-685) → GSAS | n/a |
| | | A942P, V987P | | | |
| 67 | D614G | F817P, A892P, A899P, | n/a | K854F | n/a |
| | | A942P, V987P | | | |
| 68 | D614G | F817P, A892P, A899P, | n/a | A609C-I692C | n/a |
| | | A942P, V987P | | | |
| 69 | D614G | F817P, A892P, A899P, | n/a | RRAR (682-685) → GSAS | V367F |
| | | A942P, D985P, V987P | | | |
| 70 | D614G | F817P, A892P, A899P, | n/a | RRAR (682-685) → GSAS | F329S |
| | | A942P, D985P, V987P | | | |
| 71 | D614G | F817P, A892P, A899P, | n/a | RRAR (682-685) → GSAS | D568N |
| | | A942P, D985P, V987P | | | |
| 72 | D614G | F817P, A892P, A899P, | n/a | RRAR (682-685) → GSAS | E619Q |
| | | A942P, D985P, V987P | | | |
| 73 | D614G | F817P, A892P, A899P, | n/a | RRAR (682-685) → GSAS | D830N |
| | | A942P, D985P, V987P | | | |
| 74 | D614G | F817P, A892P, A899P, | n/a | RRAR (682-685) → GSAS | D839N |
| | | A942P, D985P, V987P | | | |
| 75 | D614G | F817P, A892P, A899P, | n/a | RRAR (682-685) → GSAS | D843N |
| | | A942P, D985P, V987P | | | |
| 76 | D614G | F817P, A892P, A899P, | n/a | RRAR (682-685) → GSAS | D848N |
| | | A942P, D985P, V987P | | | |
| 77 | D614G | F817P, A892P, A899P, | A944P | RRAR (682-685) → GSAS | n/a |
| | | A942P, D985P, V987P | | | |
| 78 | D614G | F817P, A892P, A899P, | S943P | RRAR (682-685) → GSAS | n/a |
| | | A942P, D985P, V987P | | | |
| 79 | D614G | F817P, A892P, A899P, | N919Y | RRAR (682-685) → GSAS | n/a |
| | | A942P, D985P, V987P | | | |
| 80 | D614G | F817P, A892P, A899P, | N856L | RRAR (682-685) → GSAS | n/a |
| | | A942P, D985P, V987P | | | |
| 81 | D614G | F817P, A892P, A899P, | L948C-G1059C | RRAR (682-685) → GSAS | n/a |
| | | A942P, D985P, V987P | | | |
| 82 | D614G | F817P, A892P, A899P, | F970C-G999C | RRAR (682-685) → GSAS | n/a |
| | | A942P, D985P, V987P | | | |
| 83 | D614G | F817P, A892P, A899P, | A972C-Q992C | RRAR (682-685) → GSAS | n/a |
| | | A942P, D985P, V987P | | | |
| 84 | D614G | F817P, A892P, A899P, | I980C-Q992C | RRAR (682-685) → GSAS | n/a |
| | | A942P, D985P, V987P | | | |
| 85 | D614G | F817P, A892P, A899P, | n/a | K854F | n/a |
| | | A942P, D985P, V987P | | | |
| 86 | D614G | F817P, A892P, A899P, | n/a | A609C-I692C | n/a |
| | | A942P, D985P, V987P | | | |
| 87 | D614G | P6 (F817P, A892P, A899P, | n/a | RRAR | n/a |
| | | A942P, K986P, V987P) | | | |
| 88 | D614G | P6 (F817P, A892P, A899P, | | RRAR (682-685) → | |
| | | A942P, K986P, V987P) | | GSAS; A570C-N960C | |
| 89 | D614G | F817P, A892P, A899P, | | A570C-N960C | |
| | | A942P, D985P, V987P | | | |
| 90 | D614G | F817P, A892P, A899P, | | RRAR (682-685) → | |
| | | A942P, D985P, V987P | | GSAS; A570C-N960C | |
| 91 | D614G | P6 (F817P, A892P, A899P, | | A570C-N960C | S383C-D985C |
| | | A942P, K986P, V987P) | | | |
| 92 | D614G | P6 (F817P, A892P, A899P, | | RRAR (682-685) → GSAS | A570C-K964C |
| | | A942P, K986P, V987P) | | | |
| 93 | D614G | P6 (F817P, A892P, A899P, | | RRAR (682-685) → GSAS | A570C-S967C |
| | | A942P, K986P, V987P) | | | |
| 94 | D614G | F817P, A892P, A899P, | | RRAR (682-685) → GSAS | A570C-K964C |
| | | A942P, D985P, V987P | | | |
| 95 | D614G | F817P, A892P, A899P, | | RRAR (682-685) → GSAS | A570C-S967C |
| | | A942P, D985P, V987P | | | |
| 96 | D614G | P6 (F817P, A892P, A899P, | | RRAR (682-685) → GSAS | T547C-N978C |
| | | A942P, K986P, V987P) | | | |
| 97 | D614G | P6 (F817P, A892P, A899P, | | RRAR (682-685) → GSAS | T547C-S982C |
| | | A942P, K986P, V987P) | | | |
| 98 | D614G | F817P, A892P, A899P, | | RRAR (682-685) → GSAS | T547C-N978C |
| | | A942P, D985P, V987P | | | |
| 99 | D614G | F817P, A892P, A899P, | | RRAR (682-685) → GSAS | T547C-S982C |
| | | A942P, D985P, V987P | | | |

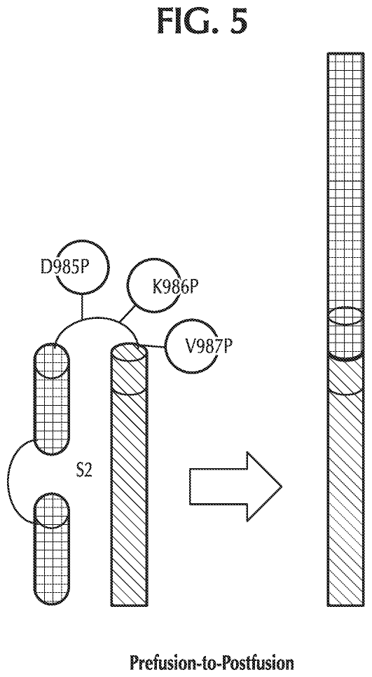

At least some variants were selected for their ability to increase the RBD-up conformation of the spike protein and/or enhance stability of the spike protein prefusion with a host cell membrane. For example, FIG. 5 illustrates the conformation of a coronavirus spike protein prefusion with a host cell membrane and post-fusion with a host cell membrane. Three amino acid residues were selected for modification. These amino acids correspond to the amino acid residues at positions *982, *983, and/or *984 of the SARS-CoV-2 spike protein sequence.

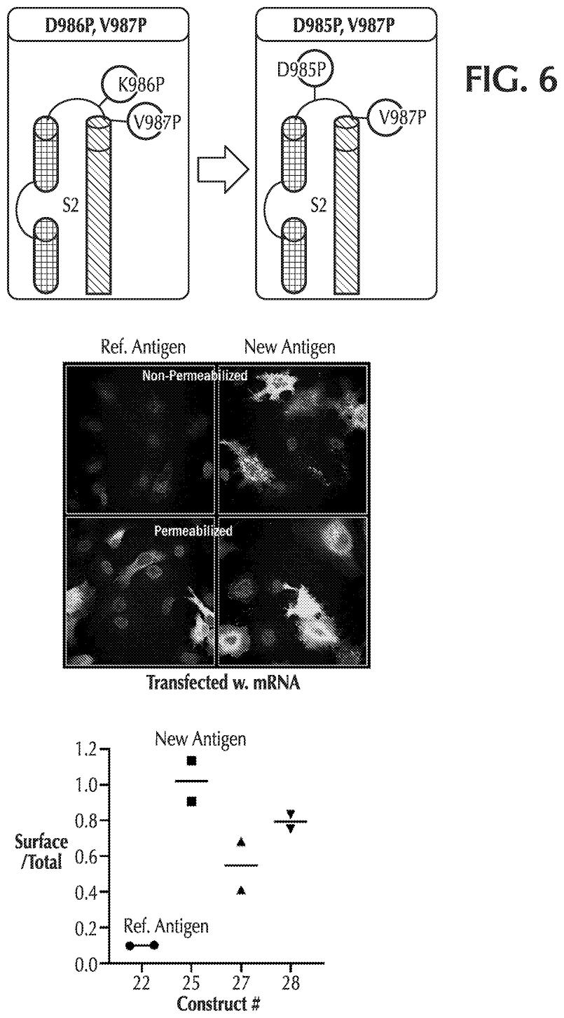

FIG. 6 demonstrates that these modifications at amino acid positions corresponding to amino acid positions *982, *983, and/or *984 of the SARS-CoV-2 spike protein can improve antigen expression and the stabilize prefusion conformation of the variant coronavirus spike protein.

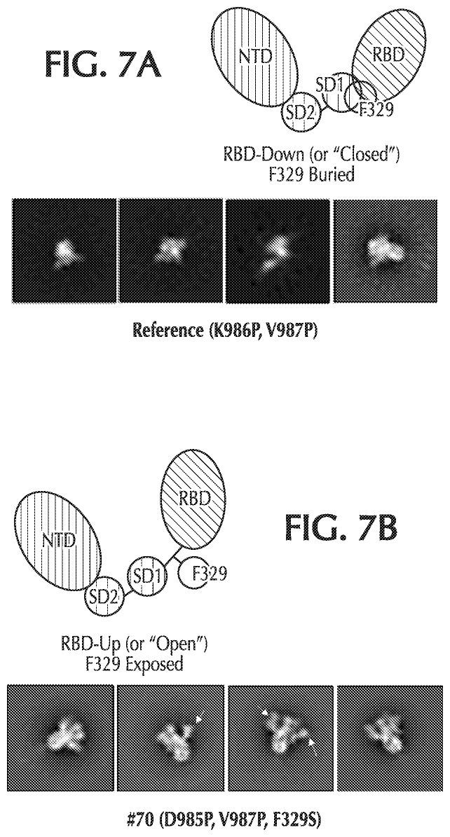

Further variants selected at least for their ability to increase the RBD-up conformation of the spike protein and/or increase adoption of a prefusion conformation include those illustrated by FIG. 7. FIG. 7 illustrates the conformation of a coronavirus spike protein prefusion with a host cell membrane (top, FIG. 7A) and post-fusion with a host cell membrane (bottom, FIG. 7B). An amino acid residue corresponding to the amino acid residue at position *326 of the SARS-CoV-2 spike protein sequence was selected for modification in addition to the amino acid residues corresponding to the amino acid residues at positions *982, *983, and/or *984 of the SARS-CoV-2 spike protein sequence.

FIG. 7 also demonstrates that these modifications at amino acid positions corresponding to amino acid positions *326, *982, *983, and/or *984 of the SARS-CoV-2 spike protein can affect the conformation assumed by the RBDs of the variant coronavirus spike protein, resulting in a greater number of RBDs in the RBD-up conformation.

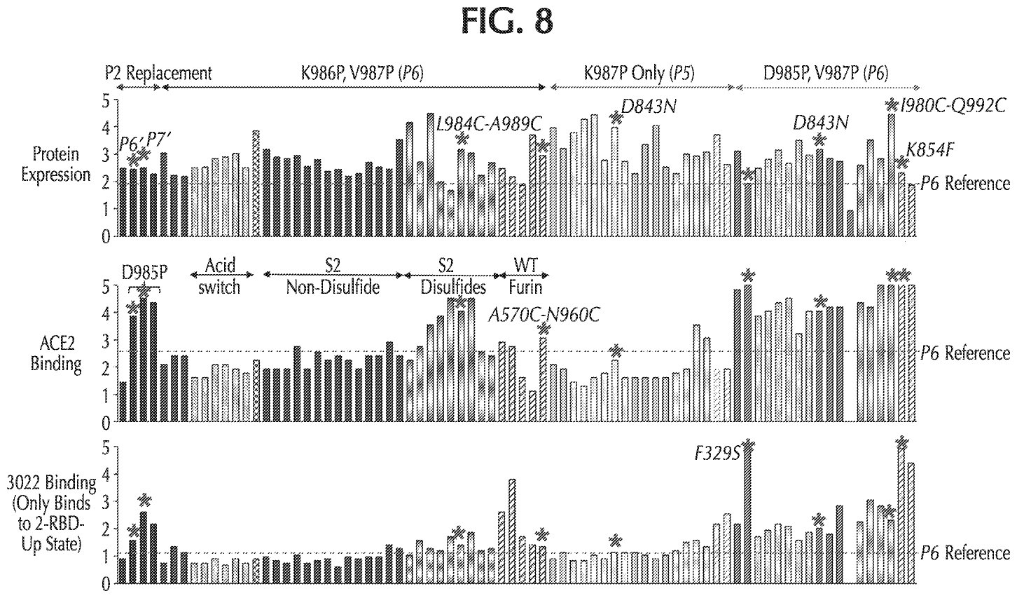

In some aspects, selected variants are screened for functional characteristics including protein expression, ACE2 binding, and 3022 binding levels. FIG. 8 provides protein expression, ACE2 binding, and 3022 binding levels of the variant coronavirus spike proteins according to some aspects disclosed herein. ACE2 is an important epitope in the coronavirus spike protein, and 3022 corresponds to a highly conserved cryptic epitope in the receptor binding domains of SARS-CoV-2 and SARS-CoV that is exposed when the RBDs of the spike proteins are in the RBD-up conformation (FIG. 2, FIG. 3). In some aspects, these measurements are utilized to select variant coronavirus spike proteins for in vivo evaluation. Results for functional characteristics including protein expression, ACE2 binding, and 3022 binding levels from the 87 mutants listed in Table 6 are shown in Table 7 below.

TABLE 7
In vitro results from mutants

| | Protein | ACE2 | 3022 |
|---|---|---|---|
| Code | Expressin | Binding | Binding |
| 01 | 2.4 | 2.4 | 1.9 |
| 02 | 1.9 | 2.7 | 1.1 |
| 03 | 2.3 | | 0.0 |
| 04 | 1.5 | 2.6 | 2.2 |
| 05 | 2.4 | 1.6 | 1.0 |
| 06 | 2.5 | 1.5 | 0.9 |
| 07 | 2.5 | 3.9 | 1.6 |
| 08 | 2.5 | 4.5 | 2.6 |
| 09 | 2.3 | 4.4 | 2.2 |
| 10 | 2.3 | 2.1 | 0.8 |
| 11 | 2.8 | 2.1 | 1.0 |
| 12 | 3.0 | 2.1 | 0.7 |
| 13 | 2.2 | 2.4 | 1.3 |
| 14 | 2.2 | 2.4 | 1.1 |
| 15 | 2.5 | 1.6 | 0.7 |
| 16 | 2.5 | 1.6 | 0.7 |
| 17 | 2.8 | 2.1 | 0.9 |
| 18 | 2.9 | 2.1 | 0.7 |
| 19 | 3.0 | 1.9 | 0.9 |
| 20 | 2.5 | 1.8 | 0.7 |
| 21 | 3.8 | 2.3 | 0.9 |
| 22 | 3.2 | 1.9 | 1.0 |
| 23 | 2.9 | 1.9 | 0.8 |
| 24 | 2.8 | 1.9 | 0.7 |
| 25 | 2.9 | 2.7 | 1.0 |
| 26 | 2.5 | 1.9 | 0.7 |
| 27 | 2.8 | 2.6 | 0.8 |
| 28 | 2.4 | 2.3 | 0.9 |
| 29 | 2.4 | 2.4 | 0.6 |
| 30 | 2.2 | 2.3 | 1.0 |
| 31 | 2.3 | 1.9 | 0.9 |
| 32 | 2.7 | 2.4 | 1.0 |
| 33 | 2.5 | 2.4 | 1.0 |
| 34 | 2.4 | 2.9 | 1.4 |
| 35 | 3.5 | 2.4 | 1.3 |
| 36 | 4.1 | 2.3 | 1.0 |
| 37 | 2.7 | 2.7 | 1.6 |
| 38 | 4.5 | 3.5 | 1.3 |
| 39 | 2.0 | 3.9 | 1.2 |
| 40 | 1.7 | 4.5 | 1.7 |
| 41 | 3.2 | 4.0 | 1.4 |
| 42 | 3.0 | 4.5 | 1.9 |
| 43 | 0.2 | 0.0 | 0.0 |
| 44 | 2.2 | 2.6 | 1.2 |
| 45 | 2.7 | 2.4 | 1.3 |
| 46 | 2.5 | 2.9 | 2.6 |
| 47 | 2.2 | 2.7 | 3.8 |
| 48 | 1.9 | 1.6 | 1.7 |
| 49 | 3.7 | 1.1 | 1.4 |
| 50 | 2.9 | 3.1 | 1.3 |
| 51 | 4.0 | 2.1 | 0.9 |
| 52 | 3.2 | 1.9 | 1.1 |
| 53 | 3.8 | 1.5 | 0.8 |
| 54 | 4.3 | 1.3 | 0.8 |
| 55 | 4.4 | 1.6 | 1.0 |
| 56 | 2.8 | 1.8 | 0.9 |
| 57 | 4.0 | 2.3 | 1.1 |
| 58 | 2.7 | 1.6 | 1.1 |
| 59 | 2.3 | 1.6 | 1.1 |
| 60 | 3.4 | 1.6 | 1.0 |
| 61 | 4.0 | 1.6 | 0.9 |
| 62 | 2.5 | 1.6 | 1.0 |
| 63 | 2.3 | 1.8 | 1.2 |
| 64 | 3.0 | 1.9 | 1.5 |
| 65 | 2.9 | 3.5 | 1.6 |
| 66 | 3.1 | 3.1 | 1.3 |
| 67 | 3.7 | 1.9 | 2.2 |
| 68 | 2.6 | 1.9 | 2.5 |
| 69 | 3.1 | 4.8 | 2.2 |
| 70 | 1.9 | 5.0 | 5.0 |
| 71 | 2.5 | 3.9 | 1.7 |
| 72 | 2.8 | 4.0 | 1.9 |
| 73 | 3.1 | 4.4 | 2.2 |
| 74 | 2.7 | 4.5 | 2.1 |
| 75 | 3.5 | 3.2 | 1.6 |
| 76 | 3.0 | 4.0 | 1.9 |
| 77 | 3.2 | 4.0 | 2.0 |
| 78 | 2.8 | 4.2 | 1.8 |
| 79 | 2.7 | 4.2 | 2.8 |
| 80 | 0.9 | 0.0 | 0.0 |
| 81 | 2.6 | 4.4 | 2.2 |
| 82 | 3.5 | 4.2 | 3.1 |
| 83 | 2.8 | 5.0 | 2.8 |
| 84 | 4.4 | 5.0 | 2.3 |
| 85 | 2.3 | 5.3 | 4.9 |
| 86 | 1.9 | 5.0 | 4.4 |
| 87 | 1.9 | 2.9 | 2.7 |

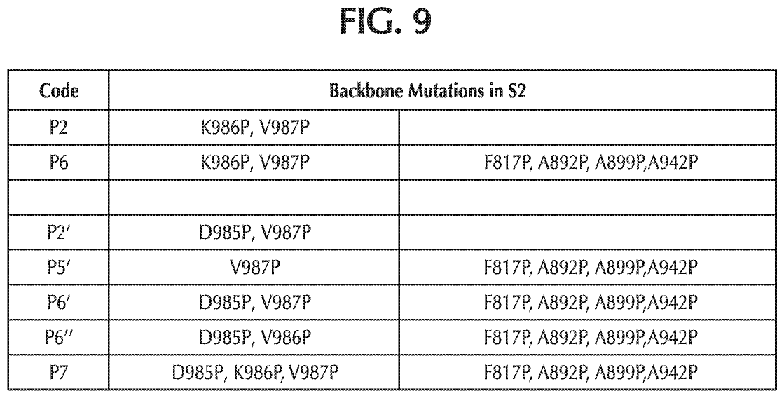

FIG. 9 provides the specific modifications made with respect to some variant spike proteins.

Mutants 70, 41, 84, 57, 75, 50, and 85 from Tables 6 and 7 were selected for immunogenicity testing in vivo. Mutant 50 showed the greatest immunogenicity in the tested group.

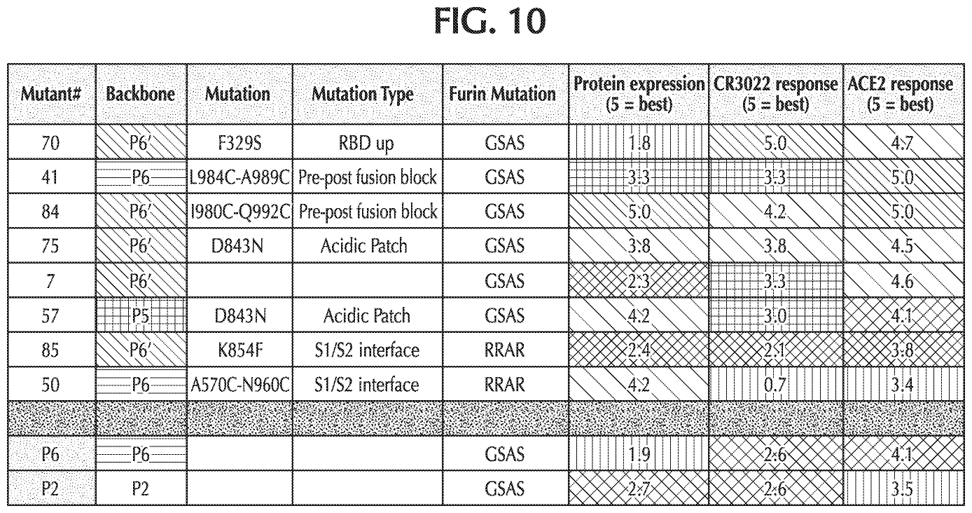

FIG. 10 describes further variants selected for in vivo immunogenicity and tolerability studies. These variants have sequence backbones with the modifications described in FIG. 9. Thus, in some aspects, amino acid residues in addition to those described in FIG. 9 are modified to produce further variant spike proteins, such as several of the variants in FIG. 10. These modifications include those amino acid residues listed in the “mutation” column in FIG. 10 and refer to the amino acids at positions corresponding to amino acids of the SARS-CoV-2 spike protein. These effect of these modifications in combination with those described in FIG. 9 on protein expression, 3022 binding, and ACE2 binding by the further variant coronavirus spike proteins is enumerated in the “Protein expression,” (CR3022 response,” and “ACE2 response” columns, respectively, in FIG. 10.

The P6′ (P6-prime) construct, which includes *D985P, *V987P, *F817P, *A892P, *A899P, and *A942P mutations shows increased protein expression and increased ACE2 receptor binding over the P6 spike protein.

Additional mutants were designed with different combinations of interprotomer disulfides bonds, wherein cysteines were introduced at the corresponding residue pair positions (such as those listed below), and proline substitutions. Mutants having any one of the following five interprotomer disulfide bonds were prepared: (i) A570C-N960C; (ii) A570C-K964C; (iii) A570C-S967C; (iv) T547C-N978C; and (v) T547C-S982C.

Exemplary mutants that were designed include those listed in Table 8.

TABLE 8
Mutations

| Design | | Interprotomer | | Furin |
|---|---|---|---|---|
| # | D614G | Disulfide | Proline mutations | site (682-685) |
| 100 | D614G | A570C-N960C | F817P, A892P, A899P, A942P, | n/a |
| | | | K986P, V987P | |
| 101 | D614G | A570C-N960C | F817P, A892P, A899P, A942P, | RRAR (682-685) |
| | | | K986P, V987P | → GSAS |
| 102 | D614G | A570C-N960C | D985P, V987P, F817P, A892P, A899P, | n/a |
| | | | A942P | |
| 103 | D614G | A570C-N960C | D985P, V987P, F817P, A892P, A899P, | RRAR (682-685) |
| | | | A942P | → GSAS |
| 104 | D614G | A570C-N960C, | F817P, A892P, A899P, A942P, | n/a |
| | | S383C-D985C | K986P, V987P | |
| 105 | D614G | A570C-K964C | F817P, A892P, A899P, A942P, | RRAR (682-685) |
| | | | K986P, V987P | → GSAS |
| 106 | D614G | A570C-S967C | F817P, A892P, A899P, A942P, | RRAR (682-685) |
| | | | K986P, V987P | → GSAS |
| 107 | D614G | A570C-K964C | D985P, V987P, F817P, A892P, A899P, | RRAR (682-685) |
| | | | A942P | → GSAS |
| 108 | D614G | A570C-S967C | D985P, V987P, F817P, A892P, A899P, | RRAR (682-685) |
| | | | A942P | → GSAS |
| 109 | D614G | T547C-N978C | F817P, A892P, A899P, A942P, | RRAR (682-685) |
| | | | K986P, V987P | → GSAS |
| 110 | D614G | T547C-S982C | F817P, A892P, A899P, A942P, | RRAR (682-685) |
| | | | K986P, V987P | → GSAS |
| 111 | D614G | T547C-N978C | D985P, V987P, F817P, A892P, A899P, | RRAR (682-685) |
| | | | A942P | → GSAS |
| 112 | D614G | T547C-S982C | D985P, V987P, F817P, A892P, A899P, | RRAR (682-685) |
| | | | A942P | → GSAS |

Immunogenicity studies were conducted to test Designs 100-112 listed in Table 8. Design 109 and Design 110 of Table 8 are preferred embodiments.

### Example 2: Immunogenicity of Variants

This study was conducted to evaluate the immunogenicity of new modified mRNA construct designs of the protein mutants described in Table 6 and/or Table 8. The modRNAs included Ni-methylpseudouracil and were encapsulated into lipid nanoparticles (LNPs). Groups containing 10 mice each were immunized with LNP formulations as a two-dose series. Sera were collected at Day 29 and Day 49 for neutralizing antibody responses. Interprotomer A570C-N960C Showed Immunogenicity

7-Day PD2: GMT across against SARS-CoV-2 variants

| Mutant # | Wuhan | Beta | Delta | BA.1 | BA.2 | BA.4/5 |
|---|---|---|---|---|---|---|
| 70 | 3635 | 470 | 327 | 79 | 63 | 36 |
| 41 | 14434 | 4418 | 3148 | 340 | 594 | 103 |
| 84 | 15285 | 4710 | 2191 | 269 | 392 | 78 |
| 57 | 17834 | 6698 | 6807 | 678 | 792 | 239 |
| 75 | 9973 | 2354 | 2431 | 113 | 251 | 63 |
| 50 | 27187 | 9658 | 8558 | 1049 | 1251 | 515 |
| 85 | 5231 | 2143 | 1140 | 223 | 360 | 44 |

1-month PD2: GMT across vaccines against SARS-CoV-2 variants

| Design # | Wuhan | Delta | BA.1 | BA.2 | BA.2.12.1 | BA.4/5 | BA.2.75.2 | BQ.1.1 | XBB |
|---|---|---|---|---|---|---|---|---|---|
| 100 | 5001 | 3879 | 1168 | 13095 | 16886 | 28950 | 981 | 7644 | 629 |
| 101 | 6605 | 5844 | 1756 | 9416 | 12791 | 28237 | 752 | 5290 | 582 |
| 102 | 5325 | 2513 | 973 | 3923 | 7446 | 10518 | 750 | 4959 | 340 |
| 103 | 8945 | 3679 | 953 | 13353 | 13039 | 40928 | 1386 | 13071 | 1476 |
| 104 | 14643 | 6367 | 1522 | 7449 | 8734 | 37636 | 936 | 6807 | 665 |
| 105 | 6844 | 2206 | 800 | 9273 | 12173 | 20461 | 911 | 3676 | 1383 |
| 106 | 10534 | 5338 | 1411 | 5330 | 7664 | 28935 | 922 | 12608 | 474 |
| 107 | 4428 | 2124 | 309 | 6326 | 8584 | 18982 | 1280 | 8365 | 802 |
| 108 | 3089 | 1284 | 197 | 3181 | 6089 | 8521 | 263 | 1807 | 145 |
| 109 | 12244 | 5037 | 2061 | 15344 | 22157 | 42525 | 1766 | 9444 | 1179 |
| 110 | 7989 | 3149 | 810 | 14174 | 22171 | 42490 | 1912 | 18697 | 2852 |
| 111 | 7101 | 2596 | 2339 | 29984 | 25603 | 50252 | 1953 | 5865 | 969 |
| 112 | 3319 | 1282 | 490 | | | 12277 | 867 | 3308 | 735 |

All of the methods disclosed and claimed herein can be made and executed without undue experimentation in light of the present disclosure. While the compositions and methods of this disclosure have been described in terms of preferred aspects, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

TABLE 9
Full amino acid sequences of the respective mutants listed in Tables 6-7.

| Mutant | | |
|---|---|---|
| code | SEQ ID NO: | notation |
| 01 | SEQ ID NO: | Signal sequence (not present in final protein product): 1-16; Foldon: |
| | 15 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); K986P |
| | | (introduced mutation); V987P (introduced mutation) |
| 02 | SEQ ID NO: | Signal sequence (not present in final protein product): 1-16; Foldon: |
| | 16 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation) |
| 03 | SEQ ID NO: | Signal sequence (not present in final protein product): 1-16; Foldon: |
| | 17 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); S383C |
| | | (introduced mutation); D985C (introduced mutation) |
| 04 | SEQ ID NO: | Signal sequence (not present in final protein product): 1-16; Foldon: |
| | 18 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; RRAR(682-685)−>GSAS |
| | | (introduced mutations); K986P (introduced mutation); V987P |
| | | (introduced mutation) |
| 05 | SEQ ID NO: | Signal sequence (not present in final protein product): 1-16; Foldon: |
| | 19 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; RRAR(682-685)−>GSAS |
| | | (introduced mutations); F817P (introduced mutation); A892P |
| | | (introduced mutation); A899P (introduced mutation); A942P |
| | | (introduced mutation); K986P (introduced mutation); V9Signal |
| | | sequence (not present in final protein product): 1-16; Foldon: 1211- |
| | | 1237; PreScission protease recognition sequence: 1241-1248; Strep- |
| | | Tag II sequence: 1252-1259; Linker sequences: 1209-1210, 1238- |
| | | 1240, 1249-1251, 1260-1261; RRAR(682-685)−>GSAS (introduced |
| | | mutations); F817P (introduced mutation); A892P (introduced |
| | | mutation); A899P (introduced mutation); A942P (introduced |
| | | mutation); K986P (introduced mutation); V987P (introduced |
| | | mutation)87P (introduced mutation) |
| 06 | SEQ ID NO: | Signal sequence (not present in final protein product): 1-16; Foldon: |
| | 20 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); V987P |
| | | (introduced mutation) |
| 07 | SEQ ID NO: | Signal sequence (not present in final protein product): 1-16; Foldon: |
| | 21 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation) |
| 08 | SEQ ID NO: | Signal sequence (not present in final protein product): 1-16; Foldon: |
| | 22 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); K986P (introduced mutation); V987P |
| | | (introduced mutation) |
| 09 | SEQ ID NO: | Signal sequence (not present in final protein product): 1-16; Foldon: |
| | 23 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); K986P (introduced mutation) |
| 10 | SEQ ID NO: | Signal sequence (not present in final protein product): 1-16; Foldon: |
| | 24 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); S943P (introduced mutation); K986P |
| | | (introduced mutation); V987P (introduced mutation) |
| 11 | SEQ ID NO: | Signal sequence (not present in final protein product): 1-16; Foldon: |
| | 25 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A944P (introduced mutation); K986P |
| | | (introduced mutation); V987P (introduced mutation) |
| 12 | SEQ ID NO: | Signal sequence (not present in final protein product): 1-16; Foldon: |
| | 26 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); V367F |
| | | (introduced mutation) |
| 13 | SEQ ID NO: | Signal sequence (not present in final protein product): 1-16; Foldon: |
| | 27 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); F329Y |
| | | (introduced mutation) |
| 14 | SEQ ID NO: | Signal sequence (not present in final protein product): 1-16; Foldon: |
| | 28 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); F329S |
| | | (introduced mutation) |
| 15 | SEQ ID NO: | Signal sequence (not present in final protein product): 1-16; Foldon: |
| | 29 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); D586N |
| | | (introduced mutation) |
| 16 | SEQ ID NO: | Signal sequence (not present in final protein product): 1-16; Foldon: |
| | 30 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); E619Q |
| | | (introduced mutation) |
| 17 | SEQ ID NO: | Signal sequence (not present in final protein product): 1-16; Foldon: |
| | 31 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); D830N |
| | | (introduced mutation) |
| 18 | SEQ ID NO: | Signal sequence (not present in final protein product): 1-16; Foldon: |
| | 32 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); D839N |
| | | (introduced mutation) |
| 19 | SEQ ID NO: | Signal sequence (not present in final protein product): 1-16; Foldon: |
| | 33 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); D843N |
| | | (introduced mutation) |
| 20 | SEQ ID NO: | Signal sequence (not present in final protein product): 1-16; Foldon: |
| | 34 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); D848N |
| | | (introduced mutation) |
| 21 | SEQ ID NO: | Signal sequence (not present in final protein product): 1-16; Foldon: |
| | 35 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); T827C |
| | | (introduced mutation), Q949C (introduced mutation) |
| 22 | SEQ ID NO: | Signal sequence (not present in final protein product): 1-16; Foldon: |
| | 36 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); A944P |
| | | (introduced mutation) |
| 23 | SEQ ID NO: | Signal sequence (not present in final protein product): 1-16; Foldon: |
| | 37 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); S943P |
| | | (introduced mutation) |
| 24 | SEQ ID NO: | Signal sequence (not present in final protein product): 1-16; Foldon: |
| | 38 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); N919Y |
| | | (introduced mutation) |
| 25 | SEQ ID NO: | Signal sequence (not present in final protein product): 1-16; Foldon: |
| | 39 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); L981Y |
| | | (introduced mutation) |
| 26 | SEQ ID NO: | Signal sequence (not present in final protein product): 1-16; Foldon: |
| | 40 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); V1061I |
| | | (introduced mutation) |
| 27 | SEQ ID NO: | Signal sequence (not present in final protein product): 1-16; Foldon: |
| | 41 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); S975P |
| | | (introduced mutation) |
| 28 | SEQ ID NO: | Signal sequence (not present in final protein product): 1-16; Foldon: |
| | 42 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); N969P |
| | | (introduced mutation) |
| 29 | SEQ ID NO: | Signal sequence (not present in final protein product): 1-16; Foldon: |
| | 43 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); N955L |
| | | (introduced mutation) |
| 30 | SEQ ID NO: | Signal sequence (not present in final protein product): 1-16; Foldon: |
| | 44 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); L938F |
| | | (introduced mutations) |
| 31 | SEQ ID NO: | Signal sequence (not present in final protein product): 1-16; Foldon: |
| | 45 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); N856L |
| | | (introduced mutation) |
| 32 | SEQ ID NO: | Signal sequence (not present in final protein product): 1-16; Foldon: |
| | 46 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); R815M |
| | | (introduced mutation) |
| 33 | SEQ ID NO: | Signal sequence (not present in final protein product): 1-16; Foldon: |
| | 47 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); A845P |
| | | (introduced mutation) |
| 34 | SEQ ID NO: | Signal sequence (not present in final protein product): 1-16; Foldon: |
| | 48 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); S746I |
| | | (introduced mutation) |
| 35 | SEQ ID NO: | Signal sequence (not present in final protein product): 1-16; Foldon: |
| | 49 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); T734I |
| | | (introduced mutation), Y1007F (introduced mutation) |
| 36 | SEQ ID NO: | Signal sequence (not present in final protein product): 1-16; Foldon: |
| | 50 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); L948C |
| | | (introduced mutation), G1059C (introduced mutation) |
| 37 | SEQ ID NO: | Signal sequence (not present in final protein product): 1-16; Foldon: |
| | 51 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); F970C |
| | | (introduced mutation), G999C (introduced mutation) |
| 38 | SEQ ID NO: | Signal sequence (not present in final protein product): 1-16; Foldon: |
| | 52 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); A972C |
| | | (introduced mutation); Q992C (introduced mutation) |
| 39 | SEQ ID NO: | Signal sequence (not present in final protein product): 1-16; Foldon: |
| | 53 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); I980C |
| | | (introduced mutation), Q992C (introduced mutation) |
| 40 | SEQ ID NO: | Signal sequence (not present in final protein product): 1-16; Foldon: |
| | 54 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); S967C |
| | | (introduced mutation), S975C (introduced mutation) |
| 41 | SEQ ID NO: | Signal sequence (not present in final protein product): 1-16; Foldon: |
| | 55 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); L984C |
| | | (introduced mutation), A989C (introduced mutation) |
| 42 | SEQ ID NO: | Signal sequence (not present in final protein product): 1-16; Foldon: |
| | 56 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); L984C |
| | | (introduced mutation), E988C (introduced mutation) |
| 43 | SEQ ID NO: | Signal sequence (not present in final protein product): 1-16; Foldon: |
| | 57 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); Q755C |
| | | (introduced mutation), N969C (introduced mutation) |
| 44 | SEQ ID NO: | Signal sequence (not present in final protein product): 1-16; Foldon: |
| | 58 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); T791C |
| | | (introduced mutation), A879C (introduced mutation) |
| 45 | SEQ ID NO: | Signal sequence (not present in final protein product): 1-16; Foldon: |
| | 59 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); S884C |
| | | (introduced mutation); A893C (introduced mutation) |
| 46 | SEQ ID NO: | Signal sequence (not present in final protein product): 1-16; Foldon: |
| | 60 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); (introduced mutation); A892P (introduced mutation); |
| | | A899P (introduced mutation); A942P (introduced mutation); K986P |
| | | (introduced mutation); V987P (introduced mutation); K854F |
| | | (introduced mutation) |
| 47 | SEQ ID NO: | Signal sequence (not present in final protein product): 1-16; Foldon: |
| | 61 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); F817P (introduced mutation); A892P (introduced |
| | | mutation); A899P (introduced mutation); A942P (introduced |
| | | mutation); K986P (introduced mutation); V987P (introduced |
| | | mutation); A609C (introduced mutation), I692C (introduced |
| | | mutation) |
| 48 | SEQ ID NO: | Signal sequence (not present in final protein product): 1-16; Foldon: |
| | 62 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); (introduced mutation); A892P (introduced mutation); |
| | | A899P (introduced mutation); A942P (introduced mutation); K986P |
| | | (introduced mutation); V987P (introduced mutation); K304E |
| | | (introduced mutation) |
| 49 | SEQ ID NO: | Signal sequence (not present in final protein product): 1-16; Foldon: |
| | 63 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); F817P (introduced mutation); A892P (introduced |
| | | mutation); A899P (introduced mutation); A942P (introduced |
| | | mutation); K986P (introduced mutation); V987P (introduced |
| | | mutation); D571C (introduced mutation), S967C (introduced |
| | | mutation) |
| 50 | SEQ ID NO: | Signal sequence (not present in final protein product): 1-16; Foldon: |
| | 64 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); F817P (introduced mutation); A892P (introduced |
| | | mutation); A899P (introduced mutation); A942P (introduced |
| | | mutation); K986P (introduced mutation); V987P (introduced |
| | | mutation); A570C (introduced mutation), N960C (introduced |
| | | mutation) |
| 51 | SEQ ID NO: | Signal sequence (not present in final protein product): 1-16; Foldon: |
| | 65 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); V987P |
| | | (introduced mutation); V367F (introduced mutation) |
| 52 | SEQ ID NO: | Signal sequence (not present in final protein product): 1-16; Foldon: |
| | 66 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); V987P |
| | | (introduced mutation); F329S (introduced mutation) |
| 53 | SEQ ID NO: | Signal sequence (not present in final protein product): 1-16; Foldon: |
| | 67 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); V987P |
| | | (introduced mutation); D586N (introduced mutation) |
| 54 | SEQ ID NO: | Signal sequence (not present in final protein product): 1-16; Foldon: |
| | 68 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); V987P |
| | | (introduced mutation); E619Q (introduced mutation) |
| 55 | SEQ ID NO: | Signal sequence (not present in final protein product): 1-16; Foldon: |
| | 69 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); V987P |
| | | (introduced mutation); D830N (introduced mutation) |
| 56 | SEQ ID NO: | Signal sequence (not present in final protein product): 1-16; Foldon: |
| | 70 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); V987P |
| | | (introduced mutation); D839N (introduced mutation) |
| 57 | SEQ ID NO: | Signal sequence (not present in final protein product): 1-16; Foldon: |
| | 71 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); V987P |
| | | (introduced mutation); D843N (introduced mutation) |
| 58 | SEQ ID NO: | Signal sequence (not present in final protein product): 1-16; Foldon: |
| | 72 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); V987P |
| | | (introduced mutation); D848N (introduced mutation) |
| 59 | SEQ ID NO: | Signal sequence (not present in final protein product): 1-16; Foldon: |
| | 73 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); V987P |
| | | (introduced mutation); A944P (introduced mutation) |
| 60 | SEQ ID NO: | Signal sequence (not present in final protein product): 1-16; Foldon: |
| | 74 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); V987P |
| | | (introduced mutation); S943P (introduced mutation) |
| 61 | SEQ ID NO: | Signal sequence (not present in final protein product): 1-16; Foldon: |
| | 75 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); V987P |
| | | (introduced mutation); N919Y (introduced mutation) |
| 62 | SEQ ID NO: | Signal sequence (not present in final protein product): 1-16; Foldon: |
| | 76 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); V987P |
| | | (introduced mutation); N856L (introduced mutation) |
| 63 | SEQ ID NO: | Signal sequence (not present in final protein product): 1-16; Foldon: |
| | 77 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); V987P |
| | | (introduced mutation); L948C (introduced mutation), G1059C |
| | | (introduced mutation) |
| 64 | SEQ ID NO: | Signal sequence (not present in final protein product): 1-16; Foldon: |
| | 78 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); V987P |
| | | (introduced mutation); F970C (introduced mutation), G999C |
| | | (introduced mutation) |
| 65 | SEQ ID NO: | Signal sequence (not present in final protein product): 1-16; Foldon: |
| | 79 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); V987P |
| | | (introduced mutation); A972C (introduced mutation); Q992C |
| | | (introduced mutation) |
| 66 | SEQ ID NO: | Signal sequence (not present in final protein product): 1-16; Foldon: |
| | 80 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); V987P |
| | | (introduced mutation); I980C (introduced mutation), Q992C |
| | | (introduced mutation) |
| 67 | SEQ ID NO: | Signal sequence (not present in final protein product): 1-16; Foldon: |
| | 81 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); F817P (introduced mutation); A892P (introduced |
| | | mutation); A899P (introduced mutation); A942P (introduced |
| | | mutation); V987P (introduced mutation); K854F (introduced |
| | | mutation) |
| 68 | SEQ ID NO: | Signal sequence (not present in final protein product): 1-16; Foldon: |
| | 82 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); F817P (introduced mutation); A892P (introduced |
| | | mutation); A899P (introduced mutation); A942P (introduced |
| | | mutation); V987P (introduced mutation); A609C (introduced |
| | | mutation), I692C (introduced mutation) |
| 69 | SEQ ID NO: | Signal sequence (not present in final protein product): 1-16; Foldon: |
| | 83 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); V367F |
| | | (introduced mutation) |
| 70 | SEQ ID NO: | Signal sequence (not present in final protein product): 1-16; Foldon: |
| | 84 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); F329S |
| | | (introduced mutation) |
| 71 | SEQ ID NO: | Signal sequence (not present in final protein product): 1-16; Foldon: |
| | 85 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); D586N |
| | | (introduced mutation) |
| 72 | SEQ ID NO: | Signal sequence (not present in final protein product): 1-16; Foldon: |
| | 86 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); E619Q |
| | | (introduced mutation) |
| 73 | SEQ ID NO: | Signal sequence (not present in final protein product): 1-16; Foldon: |
| | 87 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); D830N |
| | | (introduced mutation) |
| 74 | SEQ ID NO: | Signal sequence (not present in final protein product): 1-16; Foldon: |
| | 88 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); D839N |
| | | (introduced mutation) |
| 75 | SEQ ID NO: | Signal sequence (not present in final protein product): 1-16; Foldon: |
| | 89 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); D843N |
| | | (introduced mutation) |
| 76 | SEQ ID NO: | Signal sequence (not present in final protein product): 1-16; Foldon: |
| | 90 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); D848N |
| | | (introduced mutation) |
| 77 | SEQ ID NO: | Signal sequence (not present in final protein product): 1-16; Foldon: |
| | 91 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); A944P |
| | | (introduced mutation) |
| 78 | SEQ ID NO: | Signal sequence (not present in final protein product): 1-16; Foldon: |
| | 92 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); S943P |
| | | (introduced mutation) |
| 79 | SEQ ID NO: | Signal sequence (not present in final protein product): 1-16; Foldon: |
| | 93 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); N919Y |
| | | (introduced mutation) |
| 80 | SEQ ID NO: | Signal sequence (not present in final protein product): 1-16; Foldon: |
| | 94 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutaiton); V987P (introduced mutation); N856L |
| | | (introduced mutation) |
| 81 | SEQ ID NO: | Signal sequence (not present in final protein product): 1-16; Foldon: |
| | 95 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); L948C |
| | | (introduced mutation), G1059C (introduced mutation) |
| 82 | SEQ ID NO: | Signal sequence (not present in final protein product): 1-16; Foldon: |
| | 96 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); F970C |
| | | (introduced mutation), G999C (introduced mutation) |
| 83 | SEQ ID NO: | Signal sequence (not present in final protein product): 1-16; Foldon: |
| | 97 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); A972C |
| | | (introduced mutation); Q992C (introduced mutation) |
| 84 | SEQ ID NO: | Signal sequence (not present in final protein product): 1-16; Foldon: |
| | 98 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); I980C |
| | | (introduced mutation), Q992C (introduced mutation) |
| 85 | SEQ ID NO: | Signal sequence (not present in final protein product): 1-16; Foldon: |
| | 99 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); F817P (introduced mutation); A892P (introduced |
| | | mutation); A899P (introduced mutation); A942P (introduced |
| | | mutation); D985P (introduced mutation); V987P (introduced |
| | | mutation); K854F (introduced mutation) |
| 86 | SEQ ID NO: | Signal sequence (not present in final protein product): 1-16; Foldon: |
| | 100 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); F817P (introduced mutation); A892P (introduced |
| | | mutation); A899P (introduced mutation); A942P (introduced |
| | | mutation); D985P (introduced mutation); V987P (introduced |
| | | mutation); A609C (introduced mutation), I692C (introduced |
| | | mutation) |
| 87 | SEQ ID NO: | Signal sequence (not present in final protein product): 1-16; Foldon: |
| | 101 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); F817P (introduced mutation); A892P (introduced |
| | | mutation); A899P (introduced mutation); A942P (introduced |
| | | mutation); K986P (introduced mutation); V987P (introduced |
| | | mutation) |
| 88 | SEQ ID NO: | Signal sequence (not present in final protein product): 1-16; Foldon: |
| | 102 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); A570C |
| | | (introduced mutation), N960C (introduced mutation) |
| 89 | SEQ ID NO: | Signal sequence (not present in final protein product): 1-16; Foldon: |
| | 103 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); F817P (introduced mutation); A892P (introduced |
| | | mutation); A899P (introduced mutation); A942P (introduced |
| | | mutation); D985P (introduced mutation); V987P (introduced |
| | | mutation); A570C (introduced mutation), N960C (introduced |
| | | mutation) |
| 90 | SEQ ID NO: | Signal sequence (not present in final protein product): 1-16; Foldon: |
| | 104 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); A570C |
| | | (introduced mutation), N960C (introduced mutation) |
| 91 | SEQ ID NO: | Signal sequence (not present in final protein product): 1-16; Foldon: |
| | 105 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); F817P (introduced mutation); A892P (introduced |
| | | mutation); A899P (introduced mutation); A942P (introduced |
| | | mutation); K986P (introduced mutation); V987P (introduced |
| | | mutation); A570C (introduced mutation), N960C (introduced |
| | | mutation); S383C (introduced mutation); D985C (introduced |
| | | mutation) |
| 92 | SEQ ID NO: | Signal sequence (not present in final protein product): 1-16; Foldon: |
| | 106 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); A570C |
| | | (introduced mutation), K964C (introduced mutation) |
| 93 | SEQ ID NO: | Signal sequence (not present in final protein product): 1-16; Foldon: |
| | 107 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); A570C |
| | | (introduced mutation), S967C (introduced mutation) |
| 94 | SEQ ID NO: | Signal sequence (not present in final protein product): 1-16; Foldon: |
| | 108 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); A570C |
| | | (introduced mutation), K964C (introduced mutation) |
| 95 | SEQ ID NO: | Signal sequence (not present in final protein product): 1-16; Foldon: |
| | 109 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); A570C |
| | | (introduced mutation), S967C (introduced mutation) |
| 96 | SEQ ID NO: | Signal sequence (not present in final protein product): 1-16; Foldon: |
| | 110 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); T547C |
| | | (introduced mutation), N978C (introduced mutation) |
| 97 | SEQ ID NO: | Signal sequence (not present in final protein product): 1-16; Foldon: |
| | 111 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); K986P |
| | | (introduced mutation); V987P (introduced mutation); T547C |
| | | (introduced mutation), S982C (introduced mutation) |
| 98 | SEQ ID NO: | Signal sequence (not present in final protein product): 1-16; Foldon: |
| | 112 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); T547C |
| | | (introduced mutation), N978C (introduced mutation) |
| 99 | SEQ ID NO: | Signal sequence (not present in final protein product): 1-16; Foldon: |
| | 113 | 1211-1237; PreScission protease recognition sequence: 1241-1248; |
| | | Strep-Tag II sequence: 1252-1259; Linker sequences: 1209-1210, |
| | | 1238-1240, 1249-1251, 1260-1261; D614G (naturally-occurring |
| | | substitution); RRAR(682-685)−>GSAS (introduced mutations); F817P |
| | | (introduced mutation); A892P (introduced mutation); A899P |
| | | (introduced mutation); A942P (introduced mutation); D985P |
| | | (introduced mutation); V987P (introduced mutation); T547C |
| | | (introduced mutation), S982C (introduced mutation) |

### EMBODIMENTS

The foregoing describes embodiments of the present invention along with possible alternatives. These embodiments, however, are merely for example and the invention is not restricted thereto.
1. An isolated immunogenic polypeptide comprising a variant coronavirus spike protein that is a variant of a native coronavirus spike protein or fragment thereof, the variant coronavirus spike protein having one or more modifications compared to the native coronavirus spike protein that:
(a) increase adoption by a receptor binding domain (RBD) of the variant coronavirus spike protein of an up conformation; and/or
(b) decrease adoption by a RBD of the variant coronavirus spike protein of a down conformation; and/or
(c) increase expression of the variant coronavirus spike protein compared to the native coronavirus spike protein; and/or
(d) increase adoption of a prefusion conformation; and/or
(e) decrease shedding of a S1 subunit of the variant coronavirus spike protein; and/or
(f) improve localization of the variant coronavirus spike protein to a host cell membrane.
   2. The isolated immunogenic polypeptide of Embodiment 1, wherein the variant coronavirus spike protein comprises an amino acid sequence that is at least 70% identical to an amino acid sequence of the native coronavirus spike protein.
   3. The isolated immunogenic polypeptide of Embodiment 1 or Embodiment 2, wherein the variant coronavirus spike protein comprises an amino acid sequence that is at least 80% identical to an amino acid sequence of the native coronavirus spike protein.
   4. The isolated immunogenic polypeptide of any one of Embodiments 1-3, wherein the variant coronavirus spike protein comprises an amino acid sequence that is at least 90% identical to an amino acid sequence of the native coronavirus spike protein.
   5. The isolated immunogenic polypeptide of any one of Embodiments 1-4, wherein the variant coronavirus spike protein comprises at least a RBD.
   6. The isolated immunogenic polypeptide of any one of Embodiments 1-5, wherein the variant coronavirus spike protein has one or more modifications that increase adoption by a RBD of the variant coronavirus spike protein of an up conformation.
   7. The isolated immunogenic polypeptide of Embodiment 6, wherein an increase in adoption by the RBD of the variant coronavirus spike protein of the up conformation increases ACE2 receptor binding by the RBD compared to the native coronavirus spike protein prefusion with the host cell membrane.
   8. The isolated immunogenic polypeptide of Embodiment 6 or Embodiment 7, wherein an increase in adoption by the RBD of the variant coronavirus spike protein of the up conformation prefusion with the host cell membrane stabilizes the variant coronavirus spike protein in a prefusion conformation.
   9. The isolated immunogenic polypeptide of any one of Embodiments 6-8, wherein an increase in adoption by the RBD of the variant coronavirus spike protein of the up conformation prefusion with the host cell membrane decreases shedding of a S1 subunit of the variant coronavirus spike protein.
   10. The isolated immunogenic polypeptide of any one of Embodiments 6-9, wherein the one or more modifications comprise one or more amino acid substitutions in a S1 subunit of the variant coronavirus spike protein.
   11. The isolated immunogenic polypeptide of any one of Embodiments 6-10, wherein the one or more modifications comprise one or more amino acid substitutions in an amino acid sequence linking an N-terminal domain to the RBD of the variant coronavirus spike protein.
   12. The isolated immunogenic polypeptide of any one of Embodiments 6-11, wherein the one or more modifications of the variant coronavirus spike protein comprise an amino acid substitution at an amino acid corresponding to amino acid position 326 of SEQ ID NO:1.
   13. The isolated immunogenic polypeptide of any one of Embodiments 6-12, wherein the one or more modifications of the variant coronavirus spike protein comprise substitution of an amino acid residue at an amino acid corresponding to amino acid position 326 of SEQ ID NO:1 with a serine residue.
   14. The isolated immunogenic polypeptide of any one of Embodiments 6-13, wherein the one or more modifications comprise one or more amino acid substitutions in the RBD of the variant coronavirus spike protein.
   15. The isolated immunogenic polypeptide of any one of Embodiments 6-14, wherein the one or more modifications of the variant coronavirus spike protein comprise an amino acid substitution at an amino acid corresponding to amino acid position 364 of SEQ ID NO:1.
   16. The isolated immunogenic polypeptide of any one of Embodiments 6-15, wherein the one or more modifications of the variant coronavirus spike protein comprise substitution of an amino acid residue at an amino acid corresponding to amino acid position 364 of SEQ ID NO:1 with a phenylalanine residue.
   17. The isolated immunogenic polypeptide of any one of Embodiments 6-16, wherein the one or more modifications comprise one or more amino acid substitutions in a S2 subunit of the variant coronavirus spike protein.
   18. The isolated protein of any one of Embodiments 6-17, wherein the one or more modifications comprise one or more amino acid substitutions in an amino acid sequence linking a first heptad repeat region to a second heptad repeat region of the variant coronavirus spike protein.
   19. The isolated immunogenic polypeptide of any one of Embodiments 6-18, wherein the one or more modifications of the variant coronavirus spike protein comprise:
(a) an amino acid substitution at an amino acid corresponding to amino acid position 814 of SEQ ID NO:1; and/or
(b) an amino acid substitution at an amino acid corresponding to amino acid position 889 of SEQ ID NO:1; and/or
(c) an amino acid substitution at an amino acid corresponding to amino acid position 896 of SEQ ID NO:1; and/or
(d) an amino acid substitution at an amino acid corresponding to amino acid position 939 of SEQ ID NO:1; and/or
(e) an amino acid substitution at an amino acid corresponding to amino acid position 982 of SEQ ID NO:1; and/or
(f) an amino acid substitution at an amino acid corresponding to amino acid position 983 of SEQ ID NO:1; and/or
(g) an amino acid substitution at an amino acid corresponding to amino acid position 984 of SEQ ID NO: 1.
   20. The isolated immunogenic polypeptide of any one of Embodiments 6-19, wherein the one or more modifications of the variant coronavirus spike protein comprise:
(a) substitution of an amino acid residue at an amino acid corresponding to amino acid position 814 of SEQ ID NO:1 with a proline residue; and/or
(b) substitution of an amino acid residue at an amino acid corresponding to amino acid position 889 of SEQ ID NO:1 with a proline residue; and/or
(c) substitution of an amino acid residue at an amino acid corresponding to amino acid position 896 of SEQ ID NO:1 with a proline residue; and/or
(d) substitution of an amino acid residue at an amino acid corresponding to amino acid position 939 of SEQ ID NO:1 with a proline residue; and/or
(e) substitution of an amino acid residue at an amino acid corresponding to amino acid position 982 of SEQ ID NO:1 with a proline residue; and/or
(f) substitution of an amino acid residue at an amino acid corresponding to amino acid position 983 of SEQ ID NO:1 with a proline residue; and/or
(g) substitution of an amino acid residue at an amino acid corresponding to amino acid position 984 of SEQ ID NO:1 with a proline residue.
   21. The isolated immunogenic polypeptide of any one of Embodiments 6-20, wherein the one or more modifications comprise one or more amino acid substitutions at an interface of a S1 subunit and a S2 subunit of the variant coronavirus spike protein.
   22. The isolated immunogenic polypeptide of any one of Embodiments 6-21, wherein the one or more modifications of the variant coronavirus spike protein comprise an amino acid substitution at an amino acid corresponding to amino acid position 611 of SEQ ID NO:1.
   23. The isolated immunogenic polypeptide of any one of Embodiments 6-22, wherein the one or more modifications of the variant coronavirus spike protein comprise substitution of an amino acid residue at an amino acid corresponding to amino acid position 611 of SEQ ID NO:1 with a glycine residue.
   24. The isolated immunogenic polypeptide of any one of Embodiments 1-23, wherein the variant coronavirus spike protein has one or more modifications that decrease adoption by a RBD of the variant coronavirus spike protein of a down conformation.
   25. The isolated immunogenic polypeptide of Embodiment 24, wherein a decrease in adoption by the RBD of the variant coronavirus spike protein of a down conformation prefusion with the host cell membrane increases ACE2 receptor binding by the RBD compared to the native coronavirus spike protein.
   26. The isolated immunogenic polypeptide of Embodiment 24 or Embodiment 25, wherein a decrease in adoption by the RBD of the variant coronavirus spike protein of the down conformation prefusion with the host cell membrane stabilizes the variant coronavirus spike protein in a prefusion conformation.
   27. The isolated immunogenic polypeptide of any one of Embodiments 24-26, wherein a decrease in adoption by the RBD of the variant coronavirus spike protein of the down conformation prefusion with the host cell membrane decreases shedding of a S1 subunit of the variant coronavirus spike protein.
   28. The isolated immunogenic polypeptide of any one of Embodiments 24-27, wherein the one or more modifications comprise one or more amino acid substitutions in a S1 subunit of the variant coronavirus spike protein.
   29. The isolated immunogenic polypeptide of any one of Embodiments 24-28, wherein the one or more modifications comprise one or more amino acid substitutions in an amino acid sequence linking an N-terminal domain to the RBD of the variant coronavirus spike protein.
   30. The isolated immunogenic polypeptide of any one of Embodiments 24-29, wherein the one or more modifications of the variant coronavirus spike protein comprise an amino acid substitution at an amino acid corresponding to amino acid position 326 of SEQ ID NO:1.
   31. The isolated immunogenic polypeptide of any one of Embodiments 24-30, wherein the one or more modifications of the variant coronavirus spike protein comprise substitution of an amino acid residue at an amino acid corresponding to amino acid position 326 of SEQ ID NO:1 with a serine residue.
   32. The isolated immunogenic polypeptide of any one of Embodiments 24-31, wherein the one or more modifications comprise one or more amino acid substitutions in the RBD of the variant coronavirus spike protein.
   33. The isolated immunogenic polypeptide of any one of Embodiments 24-32, wherein the one or more modifications of the variant coronavirus spike protein comprise an amino acid substitution at an amino acid corresponding to amino acid position 364 of SEQ ID NO:1.
   34. The isolated immunogenic polypeptide of any one of Embodiments 24-33, wherein the one or more modifications of the variant coronavirus spike protein comprise substitution of an amino acid residue at an amino acid corresponding to amino acid position 364 of SEQ ID NO:1 with a phenylalanine residue.
   35. The isolated immunogenic polypeptide of any one of Embodiments 24-34, wherein the one or more modifications comprise one or more amino acid substitutions in a S2 subunit of the variant coronavirus spike protein.
   36. The isolated protein of any one of Embodiments 24-35, wherein the one or more modifications comprise one or more amino acid substitutions in an amino acid sequence linking a first heptad repeat region to a second heptad repeat region of the variant coronavirus spike protein.
   37. The isolated immunogenic polypeptide of any one of Embodiments 24-36, wherein the one or more modifications of the variant coronavirus spike protein comprise:
(a) an amino acid substitution at an amino acid corresponding to amino acid position 814 of SEQ ID NO:1; and/or
(b) an amino acid substitution at an amino acid corresponding to amino acid position 889 of SEQ ID NO:1; and/or
(c) an amino acid substitution at an amino acid corresponding to amino acid position 896 of SEQ ID NO:1; and/or
(d) an amino acid substitution at an amino acid corresponding to amino acid position 939 of SEQ ID NO:1; and/or
(e) an amino acid substitution at an amino acid corresponding to amino acid position 982 of SEQ ID NO:1; and/or
(f) an amino acid substitution at an amino acid corresponding to amino acid position 983 of SEQ ID NO:1; and/or
(g) an amino acid substitution at an amino acid corresponding to amino acid position 984 of SEQ ID NO: 1.
   38. The isolated immunogenic polypeptide of any one of Embodiments 24-37, wherein the one or more modifications of the variant coronavirus spike protein comprise:
(a) substitution of an amino acid residue at an amino acid corresponding to amino acid position 814 of SEQ ID NO:1 with a proline residue; and/or
(b) substitution of an amino acid residue at an amino acid corresponding to amino acid position 889 of SEQ ID NO:1 with a proline residue; and/or
(c) substitution of an amino acid residue at an amino acid corresponding to amino acid position 896 of SEQ ID NO:1 with a proline residue; and/or
(d) substitution of an amino acid residue at an amino acid corresponding to amino acid position 939 of SEQ ID NO:1 with a proline residue; and/or
(e) substitution of an amino acid residue at an amino acid corresponding to amino acid position 982 of SEQ ID NO:1 with a proline residue; and/or
(f) substitution of an amino acid residue at an amino acid corresponding to amino acid position 983 of SEQ ID NO:1 with a proline residue; and/or
(g) substitution of an amino acid residue at an amino acid corresponding to amino acid position 984 of SEQ ID NO:1 with a proline residue.
   39. The isolated immunogenic polypeptide of any one of Embodiments 24-38, wherein the one or more modifications comprise one or more amino acid substitutions at an interface of a S1 subunit and a S2 subunit of the variant coronavirus spike protein.
   40. The isolated immunogenic polypeptide of any one of Embodiments 24-39, wherein the one or more modifications of the variant coronavirus spike protein comprise an amino acid substitution at an amino acid corresponding to amino acid position 611 of SEQ ID NO:1.
   41. The isolated immunogenic polypeptide of any one of Embodiments 24-40, wherein the one or more modifications of the variant coronavirus spike protein comprise substitution of an amino acid residue at an amino acid corresponding to amino acid position 611 of SEQ ID NO:1 with a glycine residue.
   42. The isolated immunogenic polypeptide of any one of Embodiments 1-41, wherein the variant coronavirus spike protein has one or more modifications that increase expression of the variant coronavirus spike protein compared to the native coronavirus spike protein.
   43. The isolated immunogenic polypeptide of Embodiment 42, wherein the one or more modifications comprise one or more amino acid substitutions in a S1 subunit of the variant coronavirus spike protein.
   44. The isolated immunogenic polypeptide of Embodiment 42 or Embodiment 43, wherein the one or more modifications comprise one or more amino acid substitutions in the RBD of the variant coronavirus spike protein.
   45. The isolated immunogenic polypeptide of any one of Embodiments 42-44, wherein the one or more modifications of the variant coronavirus spike protein comprise an amino acid substitution at an amino acid corresponding to amino acid position 364 of SEQ ID NO:1.
   46. The isolated immunogenic polypeptide of any one of Embodiments 42-45, wherein the one or more modifications of the variant coronavirus spike protein comprise substitution of an amino acid residue at an amino acid corresponding to amino acid position 364 of SEQ ID NO:1 with a phenylalanine residue.
   47. The isolated immunogenic polypeptide of any one of Embodiments 42-46, wherein the one or more modifications comprise one or more amino acid substitutions in a S2 subunit of the variant coronavirus spike protein.
   48. The isolated protein of any one of Embodiments 42-47, wherein the one or more modifications comprise one or more amino acid substitutions in an amino acid sequence linking a first heptad repeat region to a second heptad repeat region of the variant coronavirus spike protein.
   49. The isolated immunogenic polypeptide of any one of Embodiments 42-48, wherein the one or more modifications of the variant coronavirus spike protein comprise:
(a) an amino acid substitution at an amino acid corresponding to amino acid position 814 of SEQ ID NO:1; and/or
(b) an amino acid substitution at an amino acid corresponding to amino acid position 889 of SEQ ID NO:1; and/or
(c) an amino acid substitution at an amino acid corresponding to amino acid position 896 of SEQ ID NO:1; and/or
(d) an amino acid substitution at an amino acid corresponding to amino acid position 939 of SEQ ID NO:1; and/or
(e) an amino acid substitution at an amino acid corresponding to amino acid position 982 of SEQ ID NO:1; and/or
(f) an amino acid substitution at an amino acid corresponding to amino acid position 983 of SEQ ID NO:1; and/or
(g) an amino acid substitution at an amino acid corresponding to amino acid position 984 of SEQ ID NO: 1.
   50. The isolated immunogenic polypeptide of any one of Embodiments 42-49, wherein the one or more modifications of the variant coronavirus spike protein comprise:
(a) substitution of an amino acid residue at an amino acid corresponding to amino acid position 814 of SEQ ID NO:1 with a proline residue; and/or
(b) substitution of an amino acid residue at an amino acid corresponding to amino acid position 889 of SEQ ID NO:1 with a proline residue; and/or
(c) substitution of an amino acid residue at an amino acid corresponding to amino acid position 896 of SEQ ID NO:1 with a proline residue; and/or
(d) substitution of an amino acid residue at an amino acid corresponding to amino acid position 939 of SEQ ID NO:1 with a proline residue; and/or
(e) substitution of an amino acid residue at an amino acid corresponding to amino acid position 982 of SEQ ID NO:1 with a proline residue; and/or
(f) substitution of an amino acid residue at an amino acid corresponding to amino acid position 983 of SEQ ID NO:1 with a proline residue; and/or
(g) substitution of an amino acid residue at an amino acid corresponding to amino acid position 984 of SEQ ID NO:1 with a proline residue.
   51. The isolated immunogenic polypeptide of any one of Embodiments 1-50, wherein the variant coronavirus spike protein has one or more modifications that increase adoption of a prefusion conformation.
   52. The isolated immunogenic polypeptide of Embodiment 50, wherein stabilization of the variant coronavirus spike protein in a prefusion conformation increases ACE2 receptor binding by the RBD compared to the native coronavirus spike protein.
   53. The isolated immunogenic polypeptide of Embodiment 50 or Embodiment 52, wherein stabilization of the variant coronavirus spike protein in a prefusion conformation decreases shedding of a S1 subunit of the variant coronavirus spike protein.
   54. The isolated immunogenic polypeptide of any one of Embodiments 51-53, wherein the one or more modifications comprise one or more amino acid substitutions in a S2 subunit of the variant coronavirus spike protein.
   55. The isolated protein of any one of Embodiments 51-54, wherein the one or more modifications comprise one or more amino acid substitutions in an amino acid sequence linking a first heptad repeat region to a second heptad repeat region of the variant coronavirus spike protein.
   56. The isolated immunogenic polypeptide of any one of Embodiments 51-55, wherein the one or more modifications of the variant coronavirus spike protein comprise:
(a) an amino acid substitution at an amino acid corresponding to amino acid position 814 of SEQ ID NO:1; and/or
(b) an amino acid substitution at an amino acid corresponding to amino acid position 889 of SEQ ID NO:1; and/or
(c) an amino acid substitution at an amino acid corresponding to amino acid position 896 of SEQ ID NO:1; and/or
(d) an amino acid substitution at an amino acid corresponding to amino acid position 939 of SEQ ID NO:1; and/or
(e) an amino acid substitution at an amino acid corresponding to amino acid position 982 of SEQ ID NO:1; and/or
(f) an amino acid substitution at an amino acid corresponding to amino acid position 983 of SEQ ID NO:1; and/or
(g) an amino acid substitution at an amino acid corresponding to amino acid position 984 of SEQ ID NO: 1.
   57. The isolated immunogenic polypeptide of any one of Embodiments 51-56, wherein the one or more modifications of the variant coronavirus spike protein comprise:
(a) substitution of an amino acid residue at an amino acid corresponding to amino acid position 814 of SEQ ID NO:1 with a proline residue; and/or
(b) substitution of an amino acid residue at an amino acid corresponding to amino acid position 889 of SEQ ID NO:1 with a proline residue; and/or
(c) substitution of an amino acid residue at an amino acid corresponding to amino acid position 896 of SEQ ID NO:1 with a proline residue; and/or
(d) substitution of an amino acid residue at an amino acid corresponding to amino acid position 939 of SEQ ID NO:1 with a proline residue; and/or
(e) substitution of an amino acid residue at an amino acid corresponding to amino acid position 982 of SEQ ID NO:1 with a proline residue; and/or
(f) substitution of an amino acid residue at an amino acid corresponding to amino acid position 983 of SEQ ID NO:1 with a proline residue; and/or
(g) substitution of an amino acid residue at an amino acid corresponding to amino acid position 984 of SEQ ID NO:1 with a proline residue.
   58. The isolated immunogenic polypeptide of any one of Embodiments 1-57, wherein the variant coronavirus spike protein has one or more modifications that decrease shedding of a S1 subunit of the variant coronavirus spike protein.
   59. The isolated immunogenic polypeptide of Embodiment 58, wherein decreased shedding of the S1 subunit of the variant coronavirus spike protein increases ACE2 receptor binding by the RBD compared to the native coronavirus spike protein.
   60. The isolated immunogenic polypeptide of Embodiment 58 or Embodiment 59, wherein decreased shedding of the S1 subunit of the variant coronavirus spike protein stabilizes the variant coronavirus spike protein in a prefusion conformation.
   61. The isolated immunogenic polypeptide of any one of Embodiments 58-60, wherein the one or more modifications comprise one or more amino acid substitutions in a S2 subunit of the variant coronavirus spike protein.
   62. The isolated protein of any one of Embodiments 58-61, wherein the one or more modifications comprise one or more amino acid substitutions in an amino acid sequence linking a first heptad repeat region to a second heptad repeat region of the variant coronavirus spike protein.
   63. The isolated immunogenic polypeptide of any one of Embodiments 58-62, wherein the one or more modifications of the variant coronavirus spike protein comprise:
(a) an amino acid substitution at an amino acid corresponding to amino acid position 814 of SEQ ID NO:1; and/or
(b) an amino acid substitution at an amino acid corresponding to amino acid position 889 of SEQ ID NO:1; and/or
(c) an amino acid substitution at an amino acid corresponding to amino acid position 896 of SEQ ID NO:1; and/or
(d) an amino acid substitution at an amino acid corresponding to amino acid position 939 of SEQ ID NO:1; and/or
(e) an amino acid substitution at an amino acid corresponding to amino acid position 982 of SEQ ID NO:1; and/or
(f) an amino acid substitution at an amino acid corresponding to amino acid position 983 of SEQ ID NO:1; and/or
(g) an amino acid substitution at an amino acid corresponding to amino acid position 984 of SEQ ID NO: 1.
   64. The isolated immunogenic polypeptide of any one of Embodiments 58-63, wherein the one or more modifications of the variant coronavirus spike protein comprise:
(a) substitution of an amino acid residue at an amino acid corresponding to amino acid position 814 of SEQ ID NO:1 with a proline residue; and/or
(b) substitution of an amino acid residue at an amino acid corresponding to amino acid position 889 of SEQ ID NO:1 with a proline residue; and/or
(c) substitution of an amino acid residue at an amino acid corresponding to amino acid position 896 of SEQ ID NO:1 with a proline residue; and/or
(d) substitution of an amino acid residue at an amino acid corresponding to amino acid position 939 of SEQ ID NO:1 with a proline residue; and/or
(e) substitution of an amino acid residue at an amino acid corresponding to amino acid position 982 of SEQ ID NO:1 with a proline residue; and/or
(f) substitution of an amino acid residue at an amino acid corresponding to amino acid position 983 of SEQ ID NO:1 with a proline residue; and/or
(g) substitution of an amino acid residue at an amino acid corresponding to amino acid position 984 of SEQ ID NO:1 with a proline residue.
   65. The isolated immunogenic polypeptide comprising a variant coronavirus spike protein that is a variant of a native coronavirus spike protein or portion thereof of any one of Embodiments 1-64, the variant coronavirus spike protein having one or more modifications compared to the native coronavirus spike protein, the one or more modifications comprising:
(a) one or more amino acid substitutions in a S1 subunit of the native coronavirus spike protein; and/or
(b) one or more amino acid substitutions in an amino acid sequence linking an N-terminal domain to an RBD of the native coronavirus spike protein; and/or
(c) one or more amino acid substitutions in an RBD of the native coronavirus spike protein; and/or
(d) one or more amino acid substitutions insertions in a S2 subunit of the native coronavirus spike protein; and/or
(e) one or more amino acid substitutions insertions in an amino acid sequence linking a first heptad repeat region to a second heptad repeat region of the native coronavirus spike protein; and/or
(f) one or more amino acid substitutions at an interface of a S1 subunit and a S2 subunit of the native coronavirus spike protein.
   66. The isolated immunogenic polypeptide of Embodiment 65, wherein:
(a) the one or more amino acid substitutions in a S1 subunit of the native coronavirus spike protein comprise:
   i. an amino acid substitution at an amino acid corresponding to amino acid position 326 of SEQ ID NO:1; and/or
   ii. an amino acid substitution at an amino acid corresponding to amino acid position 364 of SEQ ID NO:1; and/or
(b) the one or more amino acid substitutions in an amino acid sequence linking an N-terminal domain to an RBD of the native coronavirus spike protein comprise an amino acid substitution at an amino acid corresponding to amino acid position 326 of SEQ ID NO:1; and/or
(c) the one or more amino acid substitutions in an RBD of the native coronavirus spike protein comprise an amino acid substitution at an amino acid corresponding to amino acid position 364 of SEQ ID NO: 1; and/or
(d) the one or more amino acid substitutions insertions in a S2 subunit of the native coronavirus spike protein comprise:
   i. an amino acid substitution at an amino acid corresponding to amino acid position 814 of SEQ ID NO:1; and/or
   ii. an amino acid substitution at an amino acid corresponding to amino acid position 889 of SEQ ID NO:1; and/or
   iii. an amino acid substitution at an amino acid corresponding to amino acid position 896 of SEQ ID NO:1; and/or
   iv. an amino acid substitution at an amino acid corresponding to amino acid position 939 of SEQ ID NO:1; and/or
   v. an amino acid substitution at an amino acid corresponding to amino acid position 982 of SEQ ID NO:1; and/or
   vi. an amino acid substitution at an amino acid corresponding to amino acid position 983 of SEQ ID NO:1; and/or
   vii. an amino acid substitution at an amino acid corresponding to amino acid position 984 of SEQ ID NO:1; and/or
(e) the one or more amino acid substitutions insertions in an amino acid sequence linking a first heptad repeat region to a second heptad repeat region of the native coronavirus spike protein comprise:
   i. an amino acid substitution at an amino acid corresponding to amino acid position 814 of SEQ ID NO:1; and/or
   ii. an amino acid substitution at an amino acid corresponding to amino acid position 889 of SEQ ID NO:1; and/or
   iii. an amino acid substitution at an amino acid corresponding to amino acid position 896 of SEQ ID NO:1; and/or
   iv. an amino acid substitution at an amino acid corresponding to amino acid position 939 of SEQ ID NO:1; and/or
   v. an amino acid substitution at an amino acid corresponding to amino acid position 982 of SEQ ID NO:1; and/or
   vi. an amino acid substitution at an amino acid corresponding to amino acid position 983 of SEQ ID NO:1; and/or
   vii. an amino acid substitution at an amino acid corresponding to amino acid position 984 of SEQ ID NO:1; and/or
(f) the one or more amino acid substitutions at an interface of a S1 subunit and a S2 subunit of the native coronavirus spike protein comprise an amino acid substitution at an amino acid corresponding to amino acid position 611 of SEQ ID NO:1.
   67. The isolated immunogenic polypeptide of Embodiment 66, wherein:
(a) the one or more amino acid substitutions in a S1 subunit of the native coronavirus spike protein comprise:
   i. substitution of an amino acid residue at an amino acid corresponding to amino acid position 326 of SEQ ID NO:1 with a serine residue; and/or
   ii. substitution of an amino acid residue at an amino acid corresponding to amino acid position 364 of SEQ ID NO:1 with a phenylalanine residue; and/or
(b) the one or more amino acid substitutions in an amino acid sequence linking an N-terminal domain to an RBD of the native coronavirus spike protein comprise substitution of an amino acid residue at an amino acid corresponding to amino acid position 326 of SEQ ID NO:1 with a serine residue; and/or
(c) the one or more amino acid substitutions in an RBD of the native coronavirus spike protein comprise substitution of an amino acid residue at an amino acid corresponding to amino acid position 364 of SEQ ID NO:1 with a phenylalanine residue; and/or
(d) the one or more amino acid substitutions insertions in a S2 subunit of the native coronavirus spike protein comprise:
   i. substitution of an amino acid residue at an amino acid corresponding to amino acid position 814 of SEQ ID NO:1 with a proline residue; and/or
   ii. substitution of an amino acid residue at an amino acid corresponding to amino acid position 889 of SEQ ID NO:1 with a proline residue; and/or
   iii. substitution of an amino acid residue at an amino acid corresponding to amino acid position 896 of SEQ ID NO:1 with a proline residue; and/or
   iv. substitution of an amino acid residue at an amino acid corresponding to amino acid position 939 of SEQ ID NO:1 with a proline residue; and/or
   v. substitution of an amino acid residue at an amino acid corresponding to amino acid position 982 of SEQ ID NO:1 with a proline residue; and/or
   vi. substitution of an amino acid residue at an amino acid corresponding to amino acid position 983 of SEQ ID NO:1 with a proline residue; and/or
   vii. substitution of an amino acid residue at an amino acid corresponding to amino acid position 984 of SEQ ID NO:1 with a proline residue; and/or
(e) the one or more amino acid substitutions insertions in an amino acid sequence linking a first heptad repeat region to a second heptad repeat region of the native coronavirus spike protein comprise:
   i. substitution of an amino acid residue at an amino acid corresponding to amino acid position 814 of SEQ ID NO:1 with a proline residue; and/or
   ii. substitution of an amino acid residue at an amino acid corresponding to amino acid position 889 of SEQ ID NO:1 with a proline residue; and/or
   iii. substitution of an amino acid residue at an amino acid corresponding to amino acid position 896 of SEQ ID NO:1 with a proline residue; and/or
   iv. substitution of an amino acid residue at an amino acid corresponding to amino acid position 939 of SEQ ID NO:1 with a proline residue; and/or
   v. substitution of an amino acid residue at an amino acid corresponding to amino acid position 982 of SEQ ID NO:1 with a proline residue; and/or
   vi. substitution of an amino acid residue at an amino acid corresponding to amino acid position 983 of SEQ ID NO:1 with a proline residue; and/or
   vii. substitution of an amino acid residue at an amino acid corresponding to amino acid position 984 of SEQ ID NO:1 with a proline residue; and/or
(f) the one or more amino acid substitutions at an interface of a S1 subunit and a S2 subunit of the native coronavirus spike protein comprise substitution of an amino acid residue at an amino acid corresponding to amino acid position 611 with a glycine residue.
   68. The isolated immunogenic polypeptide comprising a variant coronavirus spike protein that is a variant of a native coronavirus spike protein or portion thereof of any one of Embodiments 1-67, the variant coronavirus spike protein having one or more modifications compared to the native coronavirus spike protein, the one or more modifications comprising:
(a) substitution of an amino acid residue at an amino acid corresponding to amino acid position 326 of SEQ ID NO:1 with a serine residue; and/or
(b) substitution of an amino acid residue at an amino acid corresponding to amino acid position 364 of SEQ ID NO:1 with a phenylalanine residue; and/or
(c) substitution of an amino acid residue at an amino acid corresponding to amino acid position 567 of SEQ ID NO:1 with a cysteine residue; and/or
(d) substitution of an amino acid residue at an amino acid corresponding to amino acid position 611 of SEQ ID NO:1 with a glycine residue; and/or
(e) substitution of an amino acid residue at an amino acid corresponding to amino acid position 814 of SEQ ID NO:1 with a proline residue; and/or
(f) substitution of an amino acid residue at an amino acid corresponding to amino acid position 840 of SEQ ID NO:1 with an asparagine residue; and/or
(g) substitution of an amino acid residue at an amino acid corresponding to amino acid position 851 of SEQ ID NO:1 with a phenylalanine residue; and/or
(h) substitution of an amino acid residue at an amino acid corresponding to amino acid position 889 of SEQ ID NO:1 with a proline residue; and/or
(i) substitution of an amino acid residue at an amino acid corresponding to amino acid position 896 of SEQ ID NO:1 with a proline residue; and/or
(j) substitution of an amino acid residue at an amino acid corresponding to amino acid position 939 of SEQ ID NO:1 with a proline residue; and/or
(k) substitution of an amino acid residue at an amino acid corresponding to amino acid position 957 of SEQ ID NO:1 with a cysteine residue; and/or
(l) substitution of an amino acid residue at an amino acid corresponding to amino acid position 977 of SEQ ID NO:1 with a cysteine residue; and/or
(m) substitution of an amino acid residue at an amino acid corresponding to amino acid position 981 of SEQ ID NO:1 with a cysteine residue; and/or
(n) substitution of an amino acid residue at an amino acid corresponding to amino acid position 982 of SEQ ID NO:1 with a proline residue; and/or
(o) substitution of an amino acid residue at an amino acid corresponding to amino acid position 983 of SEQ ID NO:1 with a proline residue; and/or
(p) substitution of an amino acid residue at an amino acid corresponding to amino acid position 984 of SEQ ID NO:1 with a proline residue; and/or
(q) substitution of an amino acid residue at an amino acid corresponding to amino acid position 986 of SEQ ID NO:1 with a cysteine residue; and/or
(r) substitution of an amino acid residue at an amino acid corresponding to amino acid position 989 of SEQ ID NO:1 with a cysteine residue.
   69. The isolated immunogenic polypeptide of Embodiment 68, the one or more modifications comprising:
(a) substitution of an amino acid residue at an amino acid corresponding to amino acid position 326 of SEQ ID NO:1 with a serine residue; and
(b) substitution of an amino acid residue at an amino acid corresponding to amino acid position 364 of SEQ ID NO:1 with a phenylalanine residue; and
(c) substitution of an amino acid residue at an amino acid corresponding to amino acid position 567 of SEQ ID NO:1 with a cysteine residue; and
(d) substitution of an amino acid residue at an amino acid corresponding to amino acid position 611 of SEQ ID NO:1 with a glycine residue; and
(e) substitution of an amino acid residue at an amino acid corresponding to amino acid position 814 of SEQ ID NO:1 with a proline residue; and
(f) substitution of an amino acid residue at an amino acid corresponding to amino acid position 840 of SEQ ID NO:1 with an asparagine residue; and
(g) substitution of an amino acid residue at an amino acid corresponding to amino acid position 851 of SEQ ID NO:1 with a phenylalanine residue; and
(h) substitution of an amino acid residue at an amino acid corresponding to amino acid position 889 of SEQ ID NO:1 with a proline residue; and
(i) substitution of an amino acid residue at an amino acid corresponding to amino acid position 896 of SEQ ID NO:1 with a proline residue; and
(j) substitution of an amino acid residue at an amino acid corresponding to amino acid position 939 of SEQ ID NO:1 with a proline residue; and
(k) substitution of an amino acid residue at an amino acid corresponding to amino acid position 957 of SEQ ID NO:1 with a cysteine residue; and
(l) substitution of an amino acid residue at an amino acid corresponding to amino acid position 977 of SEQ ID NO:1 with a cysteine residue; and
(m) substitution of an amino acid residue at an amino acid corresponding to amino acid position 981 of SEQ ID NO:1 with a cysteine residue; and
(n) substitution of an amino acid residue at an amino acid corresponding to amino acid position 982 of SEQ ID NO:1 with a proline residue; and
(o) substitution of an amino acid residue at an amino acid corresponding to amino acid position 983 of SEQ ID NO:1 with a proline residue; and
(p) substitution of an amino acid residue at an amino acid corresponding to amino acid position 984 of SEQ ID NO:1 with a proline residue; and
(q) substitution of an amino acid residue at an amino acid corresponding to amino acid position 986 of SEQ ID NO:1 with a cysteine residue; and
(r) substitution of an amino acid residue at an amino acid corresponding to amino acid position 989 of SEQ ID NO:1 with a cysteine residue.
   70. The isolated immunogenic polypeptide of Embodiment 68 or Embodiment 69, the one or more modifications comprising:
(a) substitution of an amino acid residue at an amino acid corresponding to amino acid position 982 of SEQ ID NO:1 with a proline residue; and
(b) substitution of an amino acid residue at an amino acid corresponding to amino acid position 984 of SEQ ID NO:1 with a proline residue.
   71. The isolated immunogenic polypeptide of Embodiment 68 or Embodiment 69, the one or more modifications comprising:
(a) substitution of an amino acid residue at an amino acid corresponding to amino acid position 814 of SEQ ID NO:1 with a proline residue; and
(b) substitution of an amino acid residue at an amino acid corresponding to amino acid position 889 of SEQ ID NO:1 with a proline residue; and
(c) substitution of an amino acid residue at an amino acid corresponding to amino acid position 896 of SEQ ID NO:1 with a proline residue; and
(d) substitution of an amino acid residue at an amino acid corresponding to amino acid position 939 of SEQ ID NO:1 with a proline residue; and
(e) substitution of an amino acid residue at an amino acid corresponding to amino acid position 984 of SEQ ID NO:1 with a proline residue.
   72. The isolated immunogenic polypeptide of Embodiment 68 or Embodiment 69, the one or more modifications comprising:
(a) substitution of an amino acid residue at an amino acid corresponding to amino acid position 814 of SEQ ID NO:1 with a proline residue; and
(b) substitution of an amino acid residue at an amino acid corresponding to amino acid position 889 of SEQ ID NO:1 with a proline residue; and
(c) substitution of an amino acid residue at an amino acid corresponding to amino acid position 896 of SEQ ID NO:1 with a proline residue; and
(d) substitution of an amino acid residue at an amino acid corresponding to amino acid position 939 of SEQ ID NO:1 with a proline residue; and
(e) substitution of an amino acid residue at an amino acid corresponding to amino acid position 982 of SEQ ID NO:1 with a proline residue; and
(f) substitution of an amino acid residue at an amino acid corresponding to amino acid position 984 of SEQ ID NO:1 with a proline residue.
   73. The isolated immunogenic polypeptide of Embodiment 68 or Embodiment 69, the one or more modifications comprising:
(a) substitution of an amino acid residue at an amino acid corresponding to amino acid position 814 of SEQ ID NO:1 with a proline residue; and
(b) substitution of an amino acid residue at an amino acid corresponding to amino acid position 889 of SEQ ID NO:1 with a proline residue; and
(c) substitution of an amino acid residue at an amino acid corresponding to amino acid position 896 of SEQ ID NO:1 with a proline residue; and
(d) substitution of an amino acid residue at an amino acid corresponding to amino acid position 939 of SEQ ID NO:1 with a proline residue; and
(e) substitution of an amino acid residue at an amino acid corresponding to amino acid position 982 of SEQ ID NO:1 with a proline residue; and
(f) substitution of an amino acid residue at an amino acid corresponding to amino acid position 983 of SEQ ID NO:1 with a proline residue.
   74. The isolated immunogenic polypeptide of Embodiment 68 or Embodiment 69, the one or more modifications comprising:
(a) substitution of an amino acid residue at an amino acid corresponding to amino acid position 814 of SEQ ID NO:1 with a proline residue; and
(b) substitution of an amino acid residue at an amino acid corresponding to amino acid position 889 of SEQ ID NO:1 with a proline residue; and
(c) substitution of an amino acid residue at an amino acid corresponding to amino acid position 896 of SEQ ID NO:1 with a proline residue; and
(d) substitution of an amino acid residue at an amino acid corresponding to amino acid position 939 of SEQ ID NO:1 with a proline residue; and
(e) substitution of an amino acid residue at an amino acid corresponding to amino acid position 982 of SEQ ID NO:1 with a proline residue; and
(f) substitution of an amino acid residue at an amino acid corresponding to amino acid position 983 of SEQ ID NO:1 with a proline residue; and
(g) substitution of an amino acid residue at an amino acid corresponding to amino acid position 984 of SEQ ID NO:1 with a proline residue.
   75. The isolated immunogenic polypeptide of Embodiment 68 or Embodiment 69, the one or more modifications comprising:
(a) substitution of an amino acid residue at an amino acid corresponding to amino acid position 326 of SEQ ID NO:1 with a serine residue; and
(b) substitution of an amino acid residue at an amino acid corresponding to amino acid position 982 of SEQ ID NO:1 with a proline residue; and
(c) substitution of an amino acid residue at an amino acid corresponding to amino acid position 984 of SEQ ID NO:1 with a proline residue.
   76. The isolated immunogenic polypeptide of Embodiment 68 or Embodiment 69, the one or more modifications comprising:
(a) substitution of an amino acid residue at an amino acid corresponding to amino acid position 611 of SEQ ID NO:1 with a glycine residue; and
(b) substitution of an amino acid residue at an amino acid corresponding to amino acid position 983 of SEQ ID NO:1 with a proline residue; and
(c) substitution of an amino acid residue at an amino acid corresponding to amino acid position 984 of SEQ ID NO:1 with a proline residue.
   77. The isolated immunogenic polypeptide of Embodiment 68 or Embodiment 69, the one or more modifications comprising:
(a) substitution of an amino acid residue at an amino acid corresponding to amino acid position 611 of SEQ ID NO:1 with a glycine residue; and
(b) substitution of an amino acid residue at an amino acid corresponding to amino acid position 982 of SEQ ID NO:1 with a proline residue; and substitution of an amino acid residue at an amino acid corresponding to amino acid position 984 of SEQ ID NO:1 with a proline residue.
   78. The isolated immunogenic polypeptide of Embodiment 68 or Embodiment 69, the one or more modifications comprising:
(a) substitution of an amino acid residue at an amino acid corresponding to amino acid position 611 of SEQ ID NO:1 with a glycine residue; and
(b) substitution of an amino acid residue at an amino acid corresponding to amino acid position 814 of SEQ ID NO:1 with a proline residue; and
(c) substitution of an amino acid residue at an amino acid corresponding to amino acid position 889 of SEQ ID NO:1 with a proline residue; and
(d) substitution of an amino acid residue at an amino acid corresponding to amino acid position 896 of SEQ ID NO:1 with a proline residue; and
(e) substitution of an amino acid residue at an amino acid corresponding to amino acid position 939 of SEQ ID NO:1 with a proline residue; and
(f) substitution of an amino acid residue at an amino acid corresponding to amino acid position 983 of SEQ ID NO:1 with a proline residue; and
(g) substitution of an amino acid residue at an amino acid corresponding to amino acid position 984 of SEQ ID NO:1 with a proline residue.
   79. The isolated immunogenic polypeptide of Embodiment 68 or Embodiment 69, the one or more modifications comprising:
(a) substitution of an amino acid residue at an amino acid corresponding to amino acid position 611 of SEQ ID NO:1 with a glycine residue; and
(b) substitution of an amino acid residue at an amino acid corresponding to amino acid position 814 of SEQ ID NO:1 with a proline residue; and
(c) substitution of an amino acid residue at an amino acid corresponding to amino acid position 889 of SEQ ID NO:1 with a proline residue; and
(d) substitution of an amino acid residue at an amino acid corresponding to amino acid position 896 of SEQ ID NO:1 with a proline residue; and
(e) substitution of an amino acid residue at an amino acid corresponding to amino acid position 939 of SEQ ID NO:1 with a proline residue; and
(f) substitution of an amino acid residue at an amino acid corresponding to amino acid position 982 of SEQ ID NO:1 with a proline residue; and
(g) substitution of an amino acid residue at an amino acid corresponding to amino acid position 984 of SEQ ID NO:1 with a proline residue.
   80. The isolated immunogenic polypeptide of Embodiment 68 or Embodiment 69, the one or more modifications comprising:
(a) substitution of an amino acid residue at an amino acid corresponding to amino acid position 326 of SEQ ID NO:1 with a serine residue; and
(b) substitution of an amino acid residue at an amino acid corresponding to amino acid position 814 of SEQ ID NO:1 with a proline residue; and
(c) substitution of an amino acid residue at an amino acid corresponding to amino acid position 889 of SEQ ID NO:1 with a proline residue; and
(d) substitution of an amino acid residue at an amino acid corresponding to amino acid position 896 of SEQ ID NO:1 with a proline residue; and
(e) substitution of an amino acid residue at an amino acid corresponding to amino acid position 939 of SEQ ID NO:1 with a proline residue; and
(f) substitution of an amino acid residue at an amino acid corresponding to amino acid position 982 of SEQ ID NO:1 with a proline residue; and
(g) substitution of an amino acid residue at an amino acid corresponding to amino acid position 984 of SEQ ID NO:1 with a proline residue.
   81. The isolated immunogenic polypeptide of Embodiment 68 or Embodiment 69, the one or more modifications comprising:
(a) substitution of an amino acid residue at an amino acid corresponding to amino acid position 814 of SEQ ID NO:1 with a proline residue; and
(b) substitution of an amino acid residue at an amino acid corresponding to amino acid position 889 of SEQ ID NO:1 with a proline residue; and
(c) substitution of an amino acid residue at an amino acid corresponding to amino acid position 896 of SEQ ID NO:1 with a proline residue; and
(d) substitution of an amino acid residue at an amino acid corresponding to amino acid position 939 of SEQ ID NO:1 with a proline residue; and
(e) substitution of an amino acid residue at an amino acid corresponding to amino acid position 981 of SEQ ID NO:1 with a cysteine residue; and
(f) substitution of an amino acid residue at an amino acid corresponding to amino acid position 983 of SEQ ID NO:1 with a proline residue; and
(g) substitution of an amino acid residue at an amino acid corresponding to amino acid position 984 of SEQ ID NO:1 with a proline residue; and
(h) substitution of an amino acid residue at an amino acid corresponding to amino acid position 986 of SEQ ID NO:1 with a cysteine residue.
   82. The isolated immunogenic polypeptide of Embodiment 68 or Embodiment 69, the one or more modifications comprising:
(a) substitution of an amino acid residue at an amino acid corresponding to amino acid position 814 of SEQ ID NO:1 with a proline residue; and
(b) substitution of an amino acid residue at an amino acid corresponding to amino acid position 889 of SEQ ID NO:1 with a proline residue; and
(c) substitution of an amino acid residue at an amino acid corresponding to amino acid position 896 of SEQ ID NO:1 with a proline residue; and
(d) substitution of an amino acid residue at an amino acid corresponding to amino acid position 939 of SEQ ID NO:1 with a proline residue; and
(e) substitution of an amino acid residue at an amino acid corresponding to amino acid position 977 of SEQ ID NO:1 with a cysteine residue; and
(f) substitution of an amino acid residue at an amino acid corresponding to amino acid position 982 of SEQ ID NO:1 with a proline residue; and
(g) substitution of an amino acid residue at an amino acid corresponding to amino acid position 984 of SEQ ID NO:1 with a proline residue; and
(h) substitution of an amino acid residue at an amino acid corresponding to amino acid position 989 of SEQ ID NO:1 with a cysteine residue.
   83. The isolated immunogenic polypeptide of Embodiment 68 or Embodiment 69, the one or more modifications comprising:
(a) substitution of an amino acid residue at an amino acid corresponding to amino acid position 814 of SEQ ID NO:1 with a proline residue; and
(b) substitution of an amino acid residue at an amino acid corresponding to amino acid position 840 of SEQ ID NO:1 with an asparagine residue; and
(c) substitution of an amino acid residue at an amino acid corresponding to amino acid position 889 of SEQ ID NO:1 with a proline residue; and
(d) substitution of an amino acid residue at an amino acid corresponding to amino acid position 896 of SEQ ID NO:1 with a proline residue; and
(e) substitution of an amino acid residue at an amino acid corresponding to amino acid position 939 of SEQ ID NO:1 with a proline residue; and
(f) substitution of an amino acid residue at an amino acid corresponding to amino acid position 982 of SEQ ID NO:1 with a proline residue; and
(g) substitution of an amino acid residue at an amino acid corresponding to amino acid position 984 of SEQ ID NO:1 with a proline residue.
   84. The isolated immunogenic polypeptide of Embodiment 68 or Embodiment 69, the one or more modifications comprising:
(a) substitution of an amino acid residue at an amino acid corresponding to amino acid position 814 of SEQ ID NO:1 with a proline residue; and
(b) substitution of an amino acid residue at an amino acid corresponding to amino acid position 840 of SEQ ID NO:1 with an asparagine residue; and
(c) substitution of an amino acid residue at an amino acid corresponding to amino acid position 889 of SEQ ID NO:1 with a proline residue; and
(d) substitution of an amino acid residue at an amino acid corresponding to amino acid position 896 of SEQ ID NO:1 with a proline residue; and
(e) substitution of an amino acid residue at an amino acid corresponding to amino acid position 939 of SEQ ID NO:1 with a proline residue; and
(f) substitution of an amino acid residue at an amino acid corresponding to amino acid position 984 of SEQ ID NO:1 with a proline residue.
   85. The isolated immunogenic polypeptide of Embodiment 68 or Embodiment 69, the one or more modifications comprising:
(a) substitution of an amino acid residue at an amino acid corresponding to amino acid position 814 of SEQ ID NO:1 with a proline residue; and
(b) substitution of an amino acid residue at an amino acid corresponding to amino acid position 851 of SEQ ID NO:1 with a phenylalanine residue; and
(c) substitution of an amino acid residue at an amino acid corresponding to amino acid position 889 of SEQ ID NO:1 with a proline residue; and
(d) substitution of an amino acid residue at an amino acid corresponding to amino acid position 896 of SEQ ID NO:1 with a proline residue; and
(e) substitution of an amino acid residue at an amino acid corresponding to amino acid position 939 of SEQ ID NO:1 with a proline residue; and
(f) substitution of an amino acid residue at an amino acid corresponding to amino acid position 982 of SEQ ID NO:1 with a proline residue; and
(g) substitution of an amino acid residue at an amino acid corresponding to amino acid position 984 of SEQ ID NO:1 with a proline residue.
   86. The isolated immunogenic polypeptide of Embodiment 68 or Embodiment 69, the one or more modifications comprising:
(a) substitution of an amino acid residue at an amino acid corresponding to amino acid position 570 of SEQ ID NO:1 with a cysteine residue; and
(b) substitution of an amino acid residue at an amino acid corresponding to amino acid position 814 of SEQ ID NO:1 with a proline residue; and
(c) substitution of an amino acid residue at an amino acid corresponding to amino acid position 889 of SEQ ID NO:1 with a proline residue; and
(d) substitution of an amino acid residue at an amino acid corresponding to amino acid position 896 of SEQ ID NO:1 with a proline residue; and
(e) substitution of an amino acid residue at an amino acid corresponding to amino acid position 939 of SEQ ID NO:1 with a proline residue; and
(f) substitution of an amino acid residue at an amino acid corresponding to amino acid position 957 of SEQ ID NO:1 with a cysteine residue; and
(g) substitution of an amino acid residue at an amino acid corresponding to amino acid position 983 of SEQ ID NO:1 with a proline residue; and
(h) substitution of an amino acid residue at an amino acid corresponding to amino acid position 984 of SEQ ID NO:1 with a proline residue.
   87. The isolated immunogenic polypeptide of any one of Embodiments 1-67, wherein the variant coronavirus spike protein is a variant of SEQ ID NO:1 or fragment thereof, the variant coronavirus spike protein having one or more modifications compared to SEQ ID NO: 1, and wherein the one or more modifications of the variant coronavirus spike protein comprise:
(a) one or more amino acid substitutions in an amino acid sequence corresponding to a S1 subunit of the coronavirus spike protein having the amino acid sequence of SEQ ID NO:1; and/or
(b) one or more amino acid substitutions in an amino acid sequence corresponding to an amino acid sequence linking an N-terminal domain to an RBD of the coronavirus spike protein having the amino acid sequence of SEQ ID NO:1; and/or
(c) one or more amino acid substitutions in an amino acid sequence corresponding to a RBD of the coronavirus spike protein having the amino acid sequence of SEQ ID NO:1; and/or
(d) one or more amino acid substitutions in an amino acid sequence corresponding to a S2 subunit of the coronavirus spike protein having the amino acid sequence of SEQ ID NO:1; and/or
(e) one or more amino acid substitutions in an amino acid sequence corresponding to an amino acid sequence linking a first heptad repeat region to a second heptad repeat region of the coronavirus spike protein having the amino acid sequence of SEQ ID NO:1; and/or
(f) one or more amino acid substitutions at an amino acid sequence corresponding to an interface of a S1 subunit and a S2 subunit of the coronavirus spike protein having the amino acid sequence of SEQ ID NO: 1.
   88. The isolated immunogenic polypeptide of Embodiment 87, wherein:
(a) the one or more amino acid substitutions in an amino acid sequence corresponding to a S1 subunit of the coronavirus spike protein having the amino acid sequence of SEQ ID NO:1 comprise:
   i. an amino acid substitution at amino acid position 326 of SEQ ID NO:1; and/or
   ii. an amino acid substitution at amino acid position 364 of SEQ ID NO:1; and/or
(b) the one or more amino acid substitutions in an amino acid sequence corresponding to an amino acid sequence linking an N-terminal domain to an RBD of the coronavirus spike protein having the amino acid sequence of SEQ ID NO:1 comprise an amino acid substitution at amino acid position 326 of SEQ ID NO: 1; and/or
(c) the one or more amino acid substitutions in an amino acid sequence corresponding to a RBD of the coronavirus spike protein having the amino acid sequence of SEQ ID NO:1 comprise an amino acid substitution at amino acid position 364 of SEQ ID NO:1; and/or
(d) the one or more amino acid substitutions insertions in an amino acid sequence corresponding to a S2 subunit of the coronavirus spike protein having the amino acid sequence of SEQ ID NO:1 comprise:
   i. an amino acid substitution at amino acid position 814 of SEQ ID NO:1; and/or
   ii. an amino acid substitution at amino acid position 889 of SEQ ID NO:1; and/or
   iii. an amino acid substitution at amino acid position 896 of SEQ ID NO: 1; and/or
   iv. an amino acid substitution at amino acid position 939 of SEQ ID NO:1; and/or
   v. an amino acid substitution at amino acid position 982 of SEQ ID NO: 1; and/or
   vi. an amino acid substitution at amino acid position 983 of SEQ ID NO: 1; and/or
   vii. an amino acid substitution at amino acid position 984 of SEQ ID NO:1; and/or
(e) the one or more amino acid substitutions insertions in an amino acid sequence corresponding to an amino acid sequence linking a first heptad repeat region to a second heptad repeat region of the coronavirus spike protein having the amino acid sequence of SEQ ID NO:1 comprise:
   i. an amino acid substitution at amino acid position 814 of SEQ ID NO:1; and/or
   ii. an amino acid substitution at amino acid position 889 of SEQ ID NO: 1; and/or
   iii. an amino acid substitution at amino acid position 896 of SEQ ID NO: 1; and/or
   iv. an amino acid substitution at amino acid position 939 of SEQ ID NO: 1; and/or
   v. an amino acid substitution at amino acid position 982 of SEQ ID NO: 1; and/or
   vi. an amino acid substitution at amino acid position 983 of SEQ ID NO: 1; and/or
   vii. an amino acid substitution at amino acid position 984 of SEQ ID NO: 1; and/or
(f) the one or more amino acid substitutions at an amino acid sequence corresponding to an interface of a S1 subunit and a S2 subunit of the coronavirus spike protein having the amino acid sequence of SEQ ID NO:1 comprise an amino acid substitution at amino acid position 611 of SEQ ID NO: 1.
   89. The isolated immunogenic polypeptide of Embodiment 88, wherein:
(a) the one or more amino acid substitutions in an amino acid sequence corresponding to a S1 subunit of the coronavirus spike protein having the amino acid sequence of SEQ ID NO:1 comprise:
   i. substitution of amino acid position 326 of SEQ ID NO:1 with a serine residue; and/or
   ii. substitution of amino acid position 364 of SEQ ID NO:1 with a phenylalanine residue; and/or
(b) the one or more amino acid substitutions in an amino acid sequence corresponding to an amino acid sequence linking an N-terminal domain to an RBD of the coronavirus spike protein having the amino acid sequence of SEQ ID NO:1 comprise substitution of amino acid position 326 of SEQ ID NO:1 with a serine residue; and/or
(c) the one or more amino acid substitutions in an amino acid sequence corresponding to a RBD of the coronavirus spike protein having the amino acid sequence of SEQ ID NO:1 comprise substitution of amino acid position 364 of SEQ ID NO:1 with a phenylalanine residue; and/or
(d) the one or more amino acid substitutions insertions in an amino acid sequence corresponding to a S2 subunit of the coronavirus spike protein having the amino acid sequence of SEQ ID NO:1 comprise:
   i. substitution of amino acid position 814 of SEQ ID NO:1 with a proline residue; and/or
   ii. substitution of amino acid position 889 of SEQ ID NO:1 with a proline residue; and/or
   iii. substitution of amino acid position 896 of SEQ ID NO:1 with a proline residue; and/or
   iv. substitution of amino acid position 939 of SEQ ID NO:1 with a proline residue; and/or
   v. substitution of amino acid position 982 of SEQ ID NO:1 with a proline residue; and/or
   vi. substitution of amino acid position 983 of SEQ ID NO:1 with a proline residue; and/or
   vii. substitution of amino acid position 984 of SEQ ID NO:1 with a proline residue; and/or
(e) the one or more amino acid substitutions insertions in an amino acid sequence corresponding to an amino acid sequence linking a first heptad repeat region to a second heptad repeat region of the coronavirus spike protein having the amino acid sequence of SEQ ID NO:1 comprise:
   i. substitution of amino acid position 814 of SEQ ID NO:1 with a proline residue; and/or
   ii. substitution of amino acid position 889 of SEQ ID NO:1 with a proline residue; and/or
   iii. substitution of amino acid position 896 of SEQ ID NO:1 with a proline residue; and/or
   iv. substitution of amino acid position 939 of SEQ ID NO:1 with a proline residue; and/or
   v. substitution of amino acid position 982 of SEQ ID NO:1 with a proline residue; and/or
   vi. substitution of amino acid position 983 of SEQ ID NO:1 with a proline residue; and/or
   vii. substitution of amino acid position 984 of SEQ ID NO:1 with a proline residue; and/or
(f) the one or more amino acid substitutions at an amino acid sequence corresponding to an interface of a S1 subunit and a S2 subunit of the coronavirus spike protein having the amino acid sequence of SEQ ID NO:1 comprise substitution of amino acid position 611 of SEQ ID NO:1 with a glycine residue.
   90. The isolated immunogenic polypeptide of any one of Embodiments 1-89, further comprising a leader sequence having an amino acid sequence that is at least 80% identical to a leader sequence of the native coronavirus spike protein.
   91. The isolated immunogenic polypeptide of Embodiment 90, wherein the leader sequence inhibits disulfide scrambling.
   92. The isolated immunogenic polypeptide of any one of Embodiments 1-91, further comprising a transmembrane sequence having an amino acid sequence that is at least 80% identical to a transmembrane sequence of the native coronavirus spike protein.
   93. The isolated immunogenic polypeptide of Embodiment 92, wherein the transmembrane sequence extends the half-life of the isolated immunogenic polypeptide.
   94. The isolated immunogenic polypeptide of any one of Embodiments 1-93, wherein the polypeptide does not comprise an endoplasmic reticulum (ER) signal sequence.
   95. The isolated immunogenic polypeptide of Embodiment 94, wherein lack of an ER signal sequence improves localization of the variant coronavirus spike protein to the host cell membrane.
   96. The isolated immunogenic polypeptide of any one of Embodiments 1-95, further comprising a trimerization domain.
   97. The isolated immunogenic polypeptide of Embodiment 96, wherein the trimerization domain is a foldon trimerization domain.
   98. The isolated immunogenic polypeptide of any one of Embodiments 1-97, wherein the variant coronavirus spike protein comprises:
(a) a leader sequence having an amino acid sequence that is at least 80% identical to a leader sequence of the native coronavirus spike protein;
(b) a transmembrane sequence having an amino acid sequence that is at least 80% identical to a transmembrane sequence of the native coronavirus spike protein;
(c) a trimerization domain; and
(d) a RBD domain,
wherein the variant coronavirus spike protein does not comprise an ER signal sequence.
   99. The isolated immunogenic polypeptide of any one of Embodiments 1-98, wherein the variant coronavirus spike protein comprises:
(a) a leader sequence having an amino acid sequence that is at least 80% identical to a leader sequence of the native coronavirus spike protein;
(b) a transmembrane sequence having an amino acid sequence that is at least 80% identical to a transmembrane sequence of the native coronavirus spike protein;
(c) a trimerization domain;
(d) a RBD domain; and
(e) substitution of amino acid position 364 of SEQ ID NO:1 with a phenylalanine residue; and
wherein the variant coronavirus spike protein does not comprise an ER signal sequence.
   100. The isolated immunogenic polypeptide of any one of Embodiments 1-99, wherein the variant coronavirus spike protein comprises:
(a) a leader sequence having an amino acid sequence that is at least 80% identical to a leader sequence of the native coronavirus spike protein;
   (b) a transmembrane sequence having an amino acid sequence that is at least 80% identical to a transmembrane sequence of the native coronavirus spike protein;
   (c) a trimerization domain;
   (d) a RBD domain; and
   (e) an amino acid substitution at an amino acid corresponding to amino acid position 364 of SEQ ID NO: 1, and
      wherein the variant coronavirus spike protein does not comprise an ER signal sequence.
         In preferred embodiments, the isolated nucleic acid does not encode a polypeptide comprising the amino acid of SEQ ID NO: 8 having the following substitutions: D985P, V987P, F817P, A892P, A899P, A942P, and a RRAR (682-685) to GSAS substitution. In preferred embodiments, isolated nucleic acid does not encode a polypeptide comprising the amino acid sequence SEQ ID NO: 21.
         101. A plurality of isolated immunogenic polypeptides comprising variant coronavirus spike proteins that are variants of a native coronavirus spike protein or fragments thereof, wherein the isolated immunogenic polypeptide comprising the plurality of isolated immunogenic polypeptides comprises the isolated immunogenic polypeptide of any one of Embodiments 1-100, and wherein greater than at least about 25%, 30%, 35%, 40%, or 50% of the variant coronavirus spike proteins adopt an up conformation.
         102. The plurality of isolated immunogenic polypeptides of Embodiment 101, wherein greater than at least about 25% of the variant coronavirus spike proteins adopt an up conformation.
         103. A nucleic acid encoding the isolated immunogenic polypeptide of any one of Embodiments 1-100.
         104. The nucleic acid of Embodiment 103, wherein the nucleic acid is DNA.
         105. A composition comprising, in a pharmaceutically acceptable composition, an isolated immunogenic polypeptide comprising a variant of a native coronavirus spike protein according to any one of Embodiments 1-100 or the nucleic acid encoding the isolated immunogenic polypeptide according to any one of Embodiments 103-104.
         106. An immunogenic composition comprising an isolated immunogenic polypeptide comprising a variant of a native coronavirus spike protein according to any one of Embodiments 1-100.
         107. A vaccine comprising the isolated immunogenic polypeptide of any one of Embodiments 1-100.
         108. A method of making a vaccine comprising the steps of mixing the isolated immunogenic polypeptide of any one of Embodiments 1-100 to make the composition of any one of Embodiment 105 or Embodiment 106 with a pharmaceutically acceptable excipient.
         109. A method of preventing or treating coronavirus infection comprising the step of administering the vaccine of Embodiment 107 to a subject in need thereof.
         110. A use of the isolated immunogenic polypeptide of any one of Embodiments 1-100 or the composition of any one of Embodiment 105 or Embodiment 106 in the manufacture of a vaccine for treatment or prevention of coronavirus infection.
         111. A use of the isolated immunogenic polypeptide of any one of Embodiments 1-100, the nucleic acid of any one of Embodiments 103-104, or the composition of any one of Embodiment 105 or Embodiment 106 for bioinformatics analyses.
         112. An automated method of producing the isolated immunogenic polypeptide of any one of Embodiments 1-100, the nucleic acid of any one of Embodiments 103-104, or the composition of any one of Embodiment 105 or Embodiment 106.
         113. A method of synthesizing the isolated immunogenic polypeptide of any one of Embodiments 1-100, the nucleic acid of any one of Embodiments 103-104, or the composition of any one of Embodiment 105 or Embodiment 106.
         114. Formulations comprising the isolated immunogenic polypeptide of any one of Embodiments 1-100, the nucleic acid of any one of Embodiments 103-104, or the composition of any one of Embodiment 105 or Embodiment 106.
         115. A device for delivery of the isolated immunogenic polypeptide of any one of Embodiments 1-100, the nucleic acid of any one of Embodiments 103-104, or the composition of any one of Embodiment 105 or Embodiment 106.
         116. The device of Embodiment 115, wherein the device comprises a syringe.

1. An isolated immunogenic polypeptide comprising an amino acid sequence that is at least 70% identical to any one of SEQ ID NO: 15s-113.

2. The isolated immunogenic polypeptide of claim 1, wherein the one or more modifications of the variant coronavirus spike protein comprise: (a) an amino acid substitution at an amino acid corresponding to amino acid position 817 of SEQ ID NO:8; and/or (b) an amino acid substitution at an amino acid corresponding to amino acid position 892 of SEQ ID NO: 8; and/or (c) an amino acid substitution at an amino acid corresponding to amino acid position 899 of SEQ ID NO: 8; and/or (d) an amino acid substitution at an amino acid corresponding to amino acid position 942 of SEQ ID NO: 8; and/or (e) an amino acid substitution at an amino acid corresponding to amino acid position 986 of SEQ ID NO: 8; and/or (f) an amino acid substitution at an amino acid corresponding to amino acid position 987 of SEQ ID NO: 8; and/or (g) an amino acid substitution at an amino acid corresponding to amino acid position 943 of SEQ ID NO: 8; and/or (h) an amino acid substitution at an amino acid corresponding to amino acid position 944 of SEQ ID NO: 8.

3. The isolated immunogenic polypeptide of any one of claims 1-2, wherein the one or more modifications of the variant coronavirus spike protein comprise: (a) an amino acid substitution at an amino acid corresponding to amino acid position F817 of SEQ ID NO:8; and/or (b) an amino acid substitution at an amino acid corresponding to amino acid position A892 of SEQ ID NO: 8; and/or (c) an amino acid substitution at an amino acid corresponding to amino acid position A899 of SEQ ID NO: 8; and/or (d) an amino acid substitution at an amino acid corresponding to amino acid position A942 of SEQ ID NO: 8; and/or (e) an amino acid substitution at an amino acid corresponding to amino acid position K986 of SEQ ID NO: 8; and/or (f) an amino acid substitution at an amino acid corresponding to amino acid position V987 of SEQ ID NO: 8; and/or (g) an amino acid substitution at an amino acid corresponding to amino acid position S943 of SEQ ID NO: 8; and/or (h) an amino acid substitution at an amino acid corresponding to amino acid position A944 of SEQ ID NO: 8.

4. The isolated immunogenic polypeptide of any one of claims 1-3, wherein the substitution is with a proline residue.

5. The isolated immunogenic polypeptide of any one of claims 1-4, further comprising an amino acid substitution at an amino acid corresponding to amino acid position D614 of SEQ ID NO: 8.

6. The isolated immunogenic polypeptide of any one of claims 1-5, further comprising an amino acid substitution at an amino acid corresponding to amino acid positions 682-685 of SEQ ID NO: 8.

7. The isolated immunogenic polypeptide of claim 6, wherein the substitution comprises a RRAR (682-685) to GSAS substitution, corresponding to amino acid positions 682-685 of SEQ ID NO: 8.

8. The isolated immunogenic polypeptide of any one of claims 1-5, further comprising any one of the amino acid substitutions: K854F; A609C and I692C; K304E; D571C and S967C; A570C and N960C of SEQ ID NO: 8.

9. The isolated immunogenic polypeptide of claim 8, further comprising an A570C and N960C amino acid substitution of SEQ ID NO: 8.

10. The isolated immunogenic polypeptide of any one of claims 1-9, further comprising any one of the amino acid substitutions: A944P; S943P; N919Y; L981Y; V10611; S975P; N969P; N955L; L938F; N856L; R815M; A845P; S7461; T7341 and Y1007F; L948C-G1059C; F970C-G999C; A972C-Q992C; 1980C-Q992C; S967C-S975C; L984C-A989C; L984C-E988C; Q755C-N969C; T791C-A879C; S884C-A893C of SEQ ID NO: 8.

11. The isolated immunogenic polypeptide of any one of claims 1-10, further comprising any one of the amino acid substitutions: S383C-D985C; V367F; F329Y; F329S; D568N; E619Q; D830N; D839N; D843N; D848N; T827C-Q949C; S383C-D985C; A570C-K964C; A570C-S967C; A570C-K964C; A570C-S967C; T547C-N978C; T547C-S982C; T547C-N978C; T547C-S982C of SEQ ID NO: 8.

12. The isolated immunogenic polypeptide according to claim 1, comprising an amino acid sequence that is at least 80% identical to SEQ ID NO: 110.

13. The isolated immunogenic polypeptide according to claim 1, comprising an amino acid sequence that is at least 80% identical to SEQ ID NO: 111.

14. A composition comprising an isolated immunogenic polypeptide according to any one of claims 1-13.

15. A composition comprising an isolated nucleic acid encoding a polypeptide according to any one of claims 1-13.

16. The composition according to any one of claims 14-15, further comprising a lipid nanoparticle.


SEQUENCE LISTING (113 sequences)

SEQ ID NO: 1 - protein, 1270 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1353 aa, synthetic construct
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRFLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 3 - protein, 1356 aa, synthetic construct
MKLFLILLVLPLASCFFTCNSNANLSMLQLGVPDNSSTIVTGLLPTHWFCANQSTSVYSANGFFYIDVGNHRSAFALHTGYYDANQYYIYVTNEIGLNASVTLKICKFSRNTTFDFLSNASSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNGSTLVDGVSRLYQPLRLTCLWPVPGLKSSTGFVYFNATGSDVNCNGYQHNSVVDVMRYNLNFSANSLDNLKSGVIVFKTLQYDVLFYCSNSSSGVLDTTIPFGPSSQPYYCFINSTINTTHVSTFVGILPPTVREIVVARTGQFYINGFKYFDLGFIEAVNFNVTTASATDFWTVAFATFVDVLVNVSATNIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVEVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIRSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDAITQTAEAIHTVTIALNKIQDVVNQQGSALNHLTSQLRHNFQAISNSIQAIYDRLDSIQADQQVDRLITGRLAALNAFVSQVLNKYTEVRGSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTTVELQGLIDQINSTYVDLKLLNRFENYIKWPWWVWLIISVVFVVLLSLLVFCCLSTGCCGCCNCLTSSMRGCCDCGSTKLPYYEFEKVHVQ

SEQ ID NO: 4 - protein, 1356 aa, synthetic construct; identical to SEQ ID NO: 3

SEQ ID NO: 5 - protein, 1353 aa, synthetic construct
MFLILLISLPTAFAVIGDLKCTSDNINDKDTGPPPISTDTVDVTNGLGTYYVLDRVYLNTTLFLNGYYPTSGSTYRNMALKGSVLLSRLWFKPPFLSDFINGIFAKVKNTKVIKDRVMYSEFPAITIGSTFVNTSYSVVVQPRTINSTQDGDNKLQGLLEVSVCQYNMCEYPQTICHPNLGNHRKELWHLDTGVVSCLYKRNFTYDVNADYLYFHFYQEGGTFYAYFTDTGVVTKFLFNVYLGMALSHYYVMPLTCNSKLTLEYWVTPLTSRQYLLAFNQDGIIFNAEDCMSDFMSEIKCKTQSIAPPTGVYELNGYTVQPIADVYRRKPNLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIEDSVFKPRPAGVLTNHDVVYAQHCFKAPKNFCPCKLNGSCVGSGPGKNNGIGTCPAGTNYLTCDNLCTPDPITFTGTYKCPQTKSLVGIGEHCSGLAVKSDYCGGNSCTCRPQAFLGWSADSCLQGDKCNIFANFILHDVNSGLTCSTDLQKANTDIILGVCVNYDLYGILGQGIFVEVNATYYNSWQNLLYDSNGNLYGFRDYIINRTFMIRSCYSGRVSAAFHANSSEPALLFRNIKCNYVFNNSLTRQLQPINYFDSYLGCVVNAYNSTAISVQTCDLTVGSGYCVDYSKNRRSRGAITTGYRFTNFEPFTVNSVNDSLEPVGGLYEIQIPSEFTIGNMVEFIQTSSPKVTIDCAAFVCGDYAACKSQLVEYGSFCDNINAILTEVNELLDTTQLQVANSLMNGVTLSTKLKDGVNFNVDDINFSPVLGCLGSECSKASSRSAIEDLLFDKVKLSDVGFVEAYNNCTGGAEIRDLICVQSYKGIKVLPPLLSENQISGYTLAATSASLFPPWTAAAGVPFYLNVQYRINGLGVTMDVLSQNQKLIANAFNNALYAIQEGFDATNSALVKIQAVVNANAEALNNLLQQLSNRFGAISASLQEILSRLDALEAEAQIDRLINGRLTALNAYVSQQLSDSTLVKFSAAQAMEKVNECVKSQSSRINFCGNGNHIISLVQNAPYGLYFIHFSYVPTKYVTARVSPGLCIAGDRGIAPKSGYFVNVNNTWMYTGSGYYYPEPITENNVVVMSTCAVNYTKAPYVMLNTSIPNLPDFKEELDQWFKNQTSVAPDLSLDYINVTFLDLQVEMNRLQEAIKVLNQSYINLKDIGTYEYYVKWPWYVWLLICLAGVAMLVLLFFICCCTGCGTSCFKKCGGCCDDYTGYQELVIKTSHDD

SEQ ID NO: 6 - protein, 1356 aa, synthetic construct
MLLIIFILPTTLAVIGDFNCTNFAINDLNTTVPRISEYVVDVSYGLGTYYILDRVYLNTTILFTGYFPKSGANFRDLSLKGTTYLSTLWYQKPFLSDFNNGIFSRVKNTKLYVNKTLYSEFSTIVIGSVFINNSYTIVVQPHNGVLEITACQYTMCEYPHTICKSKGSSRNESWHFDKSEPLCLFKKNFTYNVSTDWLYFHFYQERGTFYAYYADSGMPTTFLFSLYLGTLLSHYYVLPLTCNAISSNTDNETLQYWVTPLSKRQYLLKFDNRGVITNAVDCSSSFFSEIQCKTKSLLPNTGVYDLSGFTVKPVATVHRRIPDLPDCDIDKWLNNFNVPSPLNWERKIFSNCNFNLSTLLRLVHTDSFSCNNFDESKIYGSCFKSIVLDKFAIPNSRRSDLQLGSSGFLQSSNYKIDTTSSSCQLYYSLPAINVTINNYNPSSWNRRYGFNNFNLSSHSVVYSRYCFSVNNTFCPCAKPSFASSCKSHKPPSASCPIGTNYRSCESTTVLDHTDWCRCSCLPDPITAYDPRSCSQKKSLVGVGEHCAGFGVDEEKCGVLDGSYNVSCLCSTDAFLGWSYDTCVSNNRCNIFSNFILNGINSGTTCSNDLLQPNTEVFTDVCVDYDLYGITGQGIFKEVSAVYYNSWQNLLYDSNGNIIGFKDFVTNKTYNIFPCYAGRVSAAFHQNASSLALLYRNLKCSYVLNNISLTTQPYFDSYLGCVFNADNLTDYSVSSCALRMGSGFCVDYNSPSSSSSRRKRRSISASYRFVTFEPFNVSFVNDSIESVGGLYEIKIPTNFTIVGQEEFIQTNSPKVTIDCSLFVCSNYAACHDLLSEYGTFCDNINSILDEVNGLLDTTQLHVADTLMQGVTLSSNLNTNLHFDVDNINFKSLVGCLGPHCGSSSRSFFEDLLFDKVKLSDVGFVEAYNNCTGGSEIRDLLCVQSFNGIKVLPPILSESQISGYTTAATVAAMFPPWSAAAGIPFSLNVQYRINGLGVTMDVLNKNQKLIATAFNNALLSIQNGFSATNSALAKIQSVVNSNAQALNSLLQQLFNKFGAISSSLQEILSRLDALEAQVQIDRLINGRLTALNAYVSQQLSDISLVKFGAALAMEKVNECVKSQSPRINFCGNGNHILSLVQNAPYGLLFMHFSYKPISFKTVLVSPGLCISGDVGIAPKQGYFIKHNDHWMFTGSSYYYPEPISDKNVVFMNTCSVNFTKAPLVYLNHSVPKLSDFESELSHWFKNQTSIAPNLTLNLHTINATFLDLYYEMNLIQESIKSLNNSYINLKDIGTYEMYVKWPWYVWLLISFSFIIFLVLLFFICCCTGCGSACFSKCHNCCDEYGGHHDFVIKTSHDD

SEQ ID NO: 7 - protein, 1255 aa, synthetic construct
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 8 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 1270 aa, synthetic construct
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 1270 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 1273 aa, synthetic construct
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 1273 aa, synthetic construct
MFVFLVLLPLVSIQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSCMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - protein, 1271 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 14 - protein, 1270 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 16 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 17 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVCPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLCPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 18 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 19 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 20 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 21 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 22 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLPPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 23 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLPPVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 24 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTAPALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 25 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTASPLGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 26 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 27 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRYPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 28 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 29 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRNIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 30 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTQVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 31 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLANAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 32 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGNCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 33 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGNIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 34 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARNLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 35 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVCLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLCDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 36 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSPLGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 37 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPPALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 38 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYEYQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 39 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDIYSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 40 - protein, 1261 aa, synthetic construct; identical to SEQ ID NO: 16

SEQ ID NO: 41 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISPVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 42 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSPFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 43 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQLAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 44 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSFSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 45 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFLGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 46 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKMSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 47 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIPARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 48 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDITECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 49 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKISVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTFVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 50 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKCQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHCVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 51 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNCGAISSVLNDILSRLDPPEAEVQIDRLITCRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 52 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGCISSVLNDILSRLDPPEAEVCIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 53 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDCLSRLDPPEAEVCIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 54 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLCSNFGAISCVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 55 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRCDPPECEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 56 - protein, 1261 aa, unidentified
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRCDPPCAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 57 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLCYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSCFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 58 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKCPPIKDFGGFNFSQILPDPSKPSKRSPIEDLLFNKVTLADAGFIKQYGDCLGDIAARDLICAQKFNGLTVLPPLLTDEMIAQYTSALLCGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 59 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITCGWTFGAGPCLQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 60 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQFFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 61 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVCVLYQGVNCTEVPVAIHADQLTPTWRVYSTGSNVFQTRAGCLIGAEHVNNSYECDIPIGAGICASYQTQTNSPRRARSVASQSCIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 62 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLESFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 63 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIACTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLCSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 64 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDICDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALCTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 65 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 66 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 67 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRNIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 68 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTQVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 69 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLANAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 70 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGNCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 71 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGNIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 72 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARNLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 73 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSPLGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 74 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPPALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 75 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYEYQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 76 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFLGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 77 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKCQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHCVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 78 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNCGAISSVLNDILSRLDKPEAEVQIDRLITCRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 79 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGCISSVLNDILSRLDKPEAEVCIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 80 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDCLSRLDKPEAEVCIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 81 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQFFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 82 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVCVLYQGVNCTEVPVAIHADQLTPTWRVYSTGSNVFQTRAGCLIGAEHVNNSYECDIPIGAGICASYQTQTNSPRRARSVASQSCIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 83 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 84 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 85 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRNIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 86 - protein, 1260 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTQVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKA

SEQ ID NO: 87 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLANAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 88 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGNCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 89 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGNIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 90 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARNLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 91 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSPLGKLQDVVNQNAQALNTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 92 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPPALGKLQDVVNQNAQALNTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 93 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYEYQKLIANQFNSAIGKIQDSLSSTPSALGKLQDVVNQNAQALNTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 94 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFLGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 95 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKCQDVVNQNAQALNTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHCVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 96 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNCGAISSVLNDILSRLPKPEAEVQIDRLITCRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 97 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGCISSVLNDILSRLPKPEAEVCIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 98 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDCLSRLPKPEAEVCIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 99 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQFFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 100 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVCVLYQGVNCTEVPVAIHADQLTPTWRVYSTGSNVFQTRAGCLIGAEHVNNSYECDIPIGAGICASYQTQTNSPRRARSVASQSCIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 101 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 102 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDICDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALCTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 103 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDICDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALCTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 104 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDICDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALCTLVKQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 105 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVCPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDICDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALCTLVKQLSSNFGAISSVLNDILSRLCPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 106 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDICDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVCQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 107 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDICDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLCSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 108 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDICDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVCQLSSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 109 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDICDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLCSNFGAISSVLNDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 110 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLCGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLCDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 111 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLCGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILCRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 112 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLCGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLCDILSRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG

SEQ ID NO: 113 - protein, 1261 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLCGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILCRLPKPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGSAWSHPQFEKAG